(19) United States
(12) Patent Application Publication
(10) Pub. No.: US 2024/0279351 A1
(43) Pub. Date: Aug. 22, 2024

(54) TARGETED ADAM17 BLOCKER COMPOUNDS, ANTI-ADAM17 ANTIBODIES, METHODS OF MAKING, AND METHODS OF USING

(71) Applicant: REGENTS OF THE UNIVERSITY OF MINNESOTA, Minneapolis, MN (US)

(72) Inventors: Bruce Walcheck, Lino Lakes, MN (US); Martin Felices, Minneapolis, MN (US); Jeffrey S. Miller, Little Canada, MN (US); Hemant K. Mishra, St. Paul, MN (US); Kristin Snyder, St. Paul, MN (US)

(21) Appl. No.: 18/570,723

(22) PCT Filed: Jun. 16, 2022
(86) PCT No.: PCT/US2022/033820

§ 371 Date: Dec. 15, 2023

Related U.S. Application Data
(60) Provisional application No. 63/211,026, filed on Jun. 16, 2021.
(60) Provisional application No. 63/211,033, filed on Jun. 16, 2021.
(60) Provisional application No. 63/211,041, filed on Jun. 16, 2021.

(51) Int. Cl. C07K 16/28; A61K 39/00

(52) U.S. Cl. CPC C07K 16/2896; A61K 2039/505; C07K 2317/31; C07K 2317/622; C07K 2317/74; C07K 2317/76

(57) ABSTRACT

Compounds that inhibit ADAM17 activity include a targeting domain that selectively binds to a target, an ADAM17-inhibiting domain, and a linker operably linking the targeting domain and the ADAM17-inhibiting domain. Compounds that bind to ADAM17 include one or more anti-ADAM17 binding domains that include one or more antibody fragments that bind to ADAM17. ADAM17-binding domains and/or ADAM17-inhibiting domains can be included in therapeutic compounds and/or compounds that detect ADAM17.

### CROSS-REFERENCE TO RELATED APPLICATION

This application claims the benefit of U.S. Provisional Patent Application No. 63/211,026, filed Jun. 16, 2021; U.S. Provisional Patent Application No. 63/211,033, filed Jun. 16, 2021; and U.S. Provisional Patent Application No. 63/211,041, filed Jun. 16, 2021, each of which is incorporated herein by reference in its entirety.

### SEQUENCE LISTING

This application contains a Sequence Listing electronically submitted to the United States Patent and Trademark Office via EFS-Web as an ASCII text file entitled “0110-000656WO01_ST25.txt” having a size of 104000 bytes and created on Jun. 16, 2022. Due to the electronic filing of the Sequence Listing, the electronically submitted Sequence Listing serves as both the paper copy required by 37 CFR § 1.821(c) and the CRF required by § 1.821(e). The information contained in the Sequence Listing is incorporated by reference herein.

### SUMMARY

This disclosure describes, in one aspect, a compound that generally includes a targeting domain that selectively binds to a target, an ADAM17-inhibiting domain, and a linker operably linking the targeting domain and the ADAM17-inhibiting domain.

In one or more embodiments, the targeting domain selectively binds to an immune cell. In one or more of these embodiments, the immune cell is a T cell, a B cell, an NK cell, a monocyte, a macrophage, a dendritic cell, a myeloid-derived suppressor cell, or a mast cell.

In one or more embodiments, the targeting domain includes an antibody or a fragment thereof. In one or more of these embodiments, the antibody fragment includes an scFv. In one or more alternative embodiments, the antibody fragment includes a single domain antibody.

In one or more embodiments, the targeting domain includes the amino acid sequence of one or more of SEQ ID NO:64, SEQ ID NO:65, SEQ ID NO:66, SEQ ID NO:68, SEQ ID NO:69, or SEQ ID NO:70.

In one or more embodiments, the compound includes a second targeting domain.

In another aspect, this disclosure describes a method of increasing NK cell proliferation. Generally, the method includes contacting any embodiment of compound summarized above with a population of NK cells under conditions effective for the NK cells to proliferate.

In another aspect, this disclosure describes a method of treating a subject having, or at risk of having, an infection Generally, the method includes administering to the subject any embodiment of compound summarized above in an amount effective to treat the infection.

In another aspect, this disclosure describes a method of treating a subject having, or at risk of having a tumor. Generally, the method includes administering to the subject any embodiment of compound summarized above in an amount effective to treat the tumor.

In another aspect, this disclosure describes a method of reducing inflammation in a subject. Generally, the method includes administering to the subject any embodiment of compound summarized above in an amount effective to reduce inflammation.

In another aspect, this disclosure describes a human IgG4 antibody, or fragment thereof, that includes at least one of SEQ ID NO:17-19 and amino acids 144-469 of SEQ ID NO:20. In one or more of these embodiments, the human IgG4 antibody, or fragment thereof, includes SEQ ID NO:20.

In another aspect, this disclosure describes a human IgG4 antibody, or fragment thereof, that includes at least one of SEQ ID NO:13-15 and amino acids 128-234 of SEQ ID NO:21. In one or more of these embodiments, the human IgG4 antibody of claim, or fragment thereof, includes SEQ ID NO:21.

In another aspect, this disclosure describes a therapeutic compound. Generally, the therapeutic compound includes an anti-ADAM17 domain that includes at least one of SEQ ID NO:13, SEQ ID NO:14, SEQ ID NO:15, SEQ ID NO:17, SEQ ID NO:18, or SEQ ID NO:19; and a targeting domain that specifically binds to a target of interest.

In another aspect, this disclosure describes a compound for detecting ADAM17. Generally, the diagnostic compound includes at least one of SEQ ID NO:13, SEQ ID NO:14, SEQ ID NO:15, SEQ ID NO:17, SEQ ID NO:18, or SEQ ID NO:19; and a detectable label.

In another aspect, this disclosure describes a canine IgG4 antibody, fragment, or isotype thereof, that includes at least one of SEQ ID NO:13-15 and amino acids 129-238 of SEQ ID NO:22. In one or more of these embodiments, the canine IgG4 antibody, fragment, or isotype thereof, includes SEQ ID NO:22.

In another aspect, this disclosure describes a canine IgG4 antibody, fragment, or isotype thereof, that includes at least one of SEQ ID NO:17-19 and amino acids 141-478 of SEQ ID NO:23. In one or more of these embodiments, the canine IgG4 antibody, fragment, or isotype thereof, includes SEQ ID NO:23.

In another aspect, this disclosure describes a therapeutic compound. Generally, the therapeutic compound includes an anti-ADAM17 domain that includes the amino acid sequence of one or more of SEQ ID NO:13, SEQ ID NO:14, SEQ ID NO:15, SEQ ID NO:17, SEQ ID NO:18, or SEQ ID NO:19; and a targeting domain that specifically binds to a target of interest.

In another aspect, this disclosure describes a compound for detecting ADAM17. Generally, the compound includes an anti-ADAM17 domain that includes the amino acid sequence of one or more of SEQ ID NO:13, SEQ ID NO:14, SEQ ID NO:15, SEQ ID NO:17, SEQ ID NO:18, or SEQ ID NO:19; a detectable label.

In another aspect, this disclosure describes a method of treating a subject having, or at risk of having, an infection by SARS-CoV-2. Generally, the method includes administering to the subject a composition that includes a compound that includes an anti-ADAM17 antibody fragment, wherein the compound is administered in an amount effective to ameliorate at least one symptom or clinical sign of SARS-CoV-2 infection. In one or more of these embodiments, the compound can be an anti-ADAM17 antibody.

In one or more embodiments, the anti-ADAM17 antibody fragment comprises the amino acid sequence of any one of SEQ ID NO:13, SEQ ID NO:14, SEQ ID NO:15, SEQ ID NO:17, SEQ ID NO:18, or SEQ ID NO: 19.

The above summary is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. Data showing anti-ADAM17 scFv blocks CD16A down regulation by human NK cells.

FIG. 2. ADAM17 blockade enhances NK cell proliferation by IL-15. Human NK cells were enriched from peripheral blood as described in Example 2. (A) NK cells were labeled with CellTrace Violet dye (Thermo Fisher Scientific, Inc., Waltham, MA) and placed in culture for seven days with rhIL-15 (10 ng/ml) and/or MEDI3622 (MedImmune LLC, Gaithersburg, MD; 5 μg/ml) and/or control human IgG1 (5 μg/ml), as indicated. Cells were then harvested and examined for CellTrace dye dilution by flow cytometry. Data are representative of three independent experiments using leukocytes from separate donors. An expansion index was calculated as described in the Methods and is the fold expansion of the overall culture for each condition based on dye dilution. Data are means±SD of three independent experiments using separate donors. Statistical significance is indicated as ***p<0.001. (B) Animal treatment schema. NSG mice were treated as described in Example 2. (C) NK cells were infused and some NSG mice were treated with rhIL-15 (5 μg), as indicated. Mouse peripheral blood was collected and the number of human CD45⁺CD56⁺CD3⁻ NK cells were enumerated by flow cytometry and are shown as NK cells/μl. Data are means±SD (n=3 to 5 mice per group). (D) Mice were administered NK cells and rhIL-15 in the presence or absence of MEDI3622. The number of human NK cells were enumerated by flow cytometry and are shown as NK cells/μl. Data are means±SD (n=4 mice per group). **p<0.01; ****p<0.0001. (E) The experiment was performed as described in panel D, but NK cells were obtained from six separate donors (panels 1-6). For panel E7, mice were treated with rhIL-15 in the presence or absence of a human IgG1 control mAb. Data are means±SD (n=4 to 7 mice per group). *p<0.05; ****p<0.0001; ns=not significant.

FIG. 3. Blocking ADAM17 increases CD62L expression by IL-15-stimulated NK cells and increase NK cell expansion in vivo. (A) NK cells were placed in culture for seven days in the presence of rhIL-15 (10 ng/ml) alone or in the presence of MEDI3622 (5 μg/ml) or an isotype-matched negative control mAb (IgG1, 5 μg/ml). CD62L levels were determined by flow cytometry. The histograms show representative data, and the bar graph shows mean±SD of 3 independent experiments using leukocytes from separate donors. The y-axis on the bar graph indicates mean fluorescence intensity. Statistical significance is indicated as ***p<0.001. (B) Mice were administered NK cells and rhIL-15 (5 g) in the presence or absence of MEDI3622 (10 mg/kg). After three weeks, mouse peripheral blood was collected and relative CD62L expression levels were determined on human CD45⁺CD56⁺CD3⁻ NK cells by flow cytometry. Two separate experiments are shown using NK cells from different donors (indicated as donor 1 and donor 2). The y-axes on the bar graphs indicate mean fluorescence intensity. Data are means±SD (n=5 mice per group). **p<0.01. (C) The experiment was performed as described in panel B, but various cell surface markers were evaluated. Data are representative of two separate experiments using NK cells from different donors. The y-axes on the bar graphs indicate mean fluorescence intensity. Data are means±SD (n=5 mice per group). *p<0.05; ns=not significant. (D) Mice were administered NK cells and rhIL-15 (5 μg) in the presence or absence of DREG200 (10 mg/kg) (left panel), DREG200 and/or MEDI3622 (10 mg/kg) (middle panel), or mouse IgG isotype-matched mAb (10 mg/kg) as a control for DREG200 (right panel). The number of human NK cells in the peripheral blood were enumerated by flow cytometry and are shown as NK cells/μl. Data are means±SD (n=4 to 5 per group). ****p<0.0001; ns=not significant.

FIG. 4. ADAM17 blockade by an IgG4 isotype mAb enhances NK cell proliferation by IL-15. Human peripheral blood mononuclear cells were labeled with CellTrace Violet dye and placed in culture for seven days with rhIL-15 (10 ng/ml) in the presence or absence of the anti-ADAM17 mAb hIgG4-B07 (human IgG4) (5 μg/ml) and/or control human IgG4 (5 μg/ml), as indicated. Cells were then harvested and examined for CellTrace dye dilution by flow cytometry. Data are representative of 3 independent experiments using leukocytes from separate donors.

FIG. 5. CD56/ADAM17 TAB induces NK cell but not T cell proliferation by IL-15. Human peripheral blood mononuclear cells were labeled with CellTrace Violet dye and placed in culture for seven days with rhIL-15 (10 ng/ml) in the presence or absence of CD56/ADAM17 TAB as indicated. Cells were then harvested and examined for CellTrace dye dilution by flow cytometry. NK cells were identified as CD56⁺CD3⁻ cells and T cells were identified as CD56⁻CD3⁺. Data are representative of three independent experiments using leukocytes from separate donors.

FIG. 6. Schematic illustration of CD56×CD156b design and mechanism of action. (A) Illustration of CD56×CD156b design and CD56 scFv and CD156b scFv controls. (B) Selective CD56×CD156b-mediated action on NK cells in the presence of antibody-bound tumor cells.

FIG. 7. Treatment of SARS-CoV-2 infected mice with MEDI3622 mAb ameliorates morbidity, but increases viral titers. (A) MEDI3622 mAb treated mice have diminished morbidity compared to saline-treated mice. (B) MEDI3622-treated mice lose significantly less weight than saline-treated controls (represented as the percentage of starting weight at time of infection for each mouse) (2-way ANOVA, *p<0.05). (C) MEDI3622-treated mice have greater relative viral burdens than saline-treated mice as measured by qPCR. (D) MEDI3622-treated mice have greater relative viral burdens than saline-treated mice as measured by endpoint titration assays. Students t test **p<0.01.

FIG. 8. Damage to lungs in MEDI3622-treated SARS-CoV-2 infected mice is significantly reduced compared to those treated with saline. (A) Histological scores from parameters shown on left were assessed on a 0-3 scale for each parameter for each mouse. (B) Total histological scores from analysis done in (A) are significantly different. Students t test **p<0.01. (C) Representative sections from each treatment group show differences in damage and infiltrate. (D) More mice in the saline-treated group (7 out of 8) had collapsed and fluid filled large bronchi (arrowheads point to examples) than in the MEDI3622-treated group (three out of 10). A single representative image from one mouse lung from each group is shown.

FIG. 9. Cytokine transcript levels of MEDI3622-treated mice are higher than or similar to those of control (saline-treated) mice during SARS-CoV-2 infection. RNA was extracted from lungs of mice infected with SARS-CoV2, described in FIG. 7. Transcript levels of genes were measured with quantitative RT-qPCR and normalized against the housekeeping gene β-actin. *p<0.05.

FIG. 10. MEDI3622 blocks soluble TNF-α production in the lung. (A) Mice were treated 48 hours prior with either saline or MEDI3622 by the intraperitoneal route, then 24 hours later injected with either saline (2 mice per route) or 1 μg/mouse E. coli LPS (three mice per route) by either the intraperitoneal or intratracheal routes. Only those mice treated intratracheally with LPS had TNF-α in BALF 24 hours after injection, and treatment with MEDI3622 blocked this detection. (B) BALFs were collected on day 4 post-infection from SARS-CoV-2 infected, saline or MEDI3622-treated mice. Infectious viral burden in MEDI3622-treated mice was slightly increased at this interval. (C) BALF from mice treated with MEDI3622 had significantly less TNF-α compared to control mice. *p<0.05.

### DETAILED DESCRIPTION OF ILLUSTRATIVE EMBODIMENTS

This disclosure describes compositions and method that involve inhibition of ADAM17 (A Disintegrin and Metalloprotease 17, also known as tumor necrosis factor-α-converting enzyme, or TACE). In various aspects, this disclosure describes polypeptides that inhibit ADAM17 including, but not limited to human antibodies that specifically bind to ADAM17 and canine antibodies that specifically bind to ADAM17. The ADAM17-inhibiting polypeptides may be incorporated into, for example, a multi-valent therapeutic compound or other compound with, for example, therapeutic, imaging, or diagnostic functionality.

ADAM17 (A Disintegrin and Metalloprotease 17, also known as tumor necrosis factor-α-converting enzyme, or TACE) is a 70-kD enzyme that belongs to the ADAM protein family of disintegrins and metalloproteases. ADAM17 is a “sheddase” that is involved in processing TNF-α at the surface of the cell by cleaving and releasing a soluble ectodomain of TNF-α from membrane-bound pro-proteins, a process known as “shedding”. ADAM17 also cleaves CD16 (FcγRIII) on the surface of NK cells, neutrophils, monocytes, and macrophages. Human CD16 binds to the Fc region of IgG1 and IgG3 antibodies. Thus, ADAM17 diminishes the ability of immune cells to recognize and eliminate cells decorated with IgG antibodies. Conversely, inhibiting ADAM17 promotes the ability of the immune cells to recognize and eliminate cells decorated with IgG antibodies.

In the context of various aspects of the technology described herein, this disclosure refers to “an antibody,” “antibodies,” and “antibody fragment.” As used herein, the term “antibody” refers generally an immunoglobulin or a fragment thereof. Thus, as used herein, the term “antibody” encompasses not only immunoglobulins with an intact Fc region, but also antibody fragments capable of binding to a biological molecule (such as an antigen or receptor) or a portion thereof, including but not limited to Fab, Fab′ and F(ab′)2, pFc′, Fd, a single domain antibody (sdAb), a variable fragment (Fv), a single-chain variable fragment (scFv) or a disulfide-linked Fv (sdFv); a diabody or a bivalent diabody; a linear antibody; a single-chain antibody molecule; and a multispecific antibody (e.g., a tribody) formed from antibody fragments. The antibody can be of any type (e.g., IgG, IgE, IgM, IgD, IgA and IgY), class (e.g., IgG1, IgG2, IgG3, IgG4, IgA1 and IgA2), or subclass.

Thus, In one or more embodiments, an antibody can be, or can include, a humanized antibody derived from an animal single domain antibody. While an scFv has a heavy variable chain component and a light variable chain component joined by a flanking sequence, a single domain antibody consists of a single monomeric variable chain—i.e., a variable heavy chin or a variable light chain—that is capable of specifically engaging a target. A single domain antibody may be derived from an antibody of any suitable animal such as, for example, a camelid (e.g., a llama or camel) or a cartilaginous fish. A single domain antibody can provide superior physical stability, an ability to bind deep grooves, and increased production yields compared to larger antibody fragments.

### Targeted ADAM17 Blocker (TAB) Compounds

In one aspect, this disclosure describes Targeted ADAM17 Blocker (TAB) compounds that generally include a domain that inhibits ADAM17 activity and at least one targeting domain that targets delivery of the TAB compound to a desired target. ADAM17 activity includes, for example, cleaving CD16 (FcγRIII) on the surface of NK cells, neutrophils, monocytes, and/or macrophages. CD16 binds to the Fc region of IgG antibodies. Thus, the TAB compounds described herein promote the ability of the immune cells to recognize and eliminate cells decorated with IgG antibodies.

Thus, in one aspect, this disclosure describes a TAB compound that includes an ADAM17-inhibiting domain, at least one targeting domain that selectively binds to a target, and a linker operably linking the targeting domain and the ADAM17-inhibiting domain.

The targeting domain can include any moiety that selectively binds to an intended target such as, for example, a tumor cell, a target in the cancer stroma, a target on an inhibitory cell such as myeloid derived suppressor cells that are CD33⁺, a virus, a bacterium, a fungus, a parasite, or a target on a cell infected by a pathogen (e.g., a virus, a bacterium, a parasite, or a fungus).

As used herein, the term “selectively binds” refers to the ability to differentiate between two or more alternatives such as, for example, having differential affinity, to any degree, for a particular target. As used herein, “antibody” refers generally to an immunoglobulin or a fragment thereof and thus encompasses a monoclonal antibody or a fragment thereof (e.g., scFv, Fab, F(ab′)₂, Fv, sdAb, or other modified forms). Thus, for brevity, reference to an antibody that selectively binds to a specified target includes any antibody fragment that exhibits the described binding character. Similarly, reference to a ligand that selectively binds to a specified target includes any fragment of the ligand that exhibits the described binding character.

Exemplary targeting domain moieties include, for example, SARS-CoV2, the causative agent of COVID-19, spike protein receptor binding domain (RBD) or another known angiotensin-converting enzyme 2 (ACE2)-targeting protein or antibody (e.g., SAR-CoV receptor binding domain (RBD) or spike protein); type-II transmembrane serine protease TMPRSS2; an NK cell-targeting antibody (e.g., an anti-CD16 antibody, an anti-CD56 antibody, an anti-NKG2D antibody, an anti-NKG2C antibody, an anti-NKp46 antibody, an anti-NKp30 antibody, an anti-NKp44 antibody, an anti-NKG2A antibody, an anti-KIR antibody, etc.); a T cell-targeting antibody (e.g., an anti-CD3, an anti-CD4, an anti-CD8, or an anti-TCRβ antibody); a B cell-targeting antibody (e.g., an anti-CD19, an anti-CD22, or an anti-CD20 antibody); an anti-CD64 antibody; an anti-CD14 antibody; and anti-CD15 antibody, and anti-CD45 antibody, an anti-L-selectin antibody; an anti-tumor antibody (e.g., an anti-CD33 antibody, an anti-CLEC12A antibody, an anti-CD19 antibody, an anti-mesothelin antibody, an anti-prostate-specific membrane antigen (PSMA) antibody, an anti-B7H3 antibody, an anti-IGF1R antibody, an anti-TEM8 antibody, an anti-ROR1 antibody, rituximab (anti-CD20), afutuzumab (anti-CD20), trastuzumab (anti-HER2/neu), pertuzumab (anti-HER2/neu), labetuzumab (anti-CEA), adecatumumab (anti-EpCAM), citatuzumab bogatox (anti-EpCAM), edrecolomab (anti-EpCAM), arcitumomab (anti-CEA), bevacizumab (anti-VEGF-A), cetuximab (anti-EGFR), nimotuzumab (anti-EGFR), panitumumab (anti-EGFR), zalutumumab (anti-EGFR), gemtuzumab ozogamicin (anti-CD33), lintuzumab (anti-CD33), etaracizumab (anti-integrin αᵥβ₃), Intetumumab (anti-CD51), ipilimumab (anti-CD152), oregovomab (anti-CA-125), votumumab (anti-tumor antigen CTAA16.88), or pemtumomab (anti-MUC1), and anti-CD133 antibody, or an anti-CSPG4 antibody).

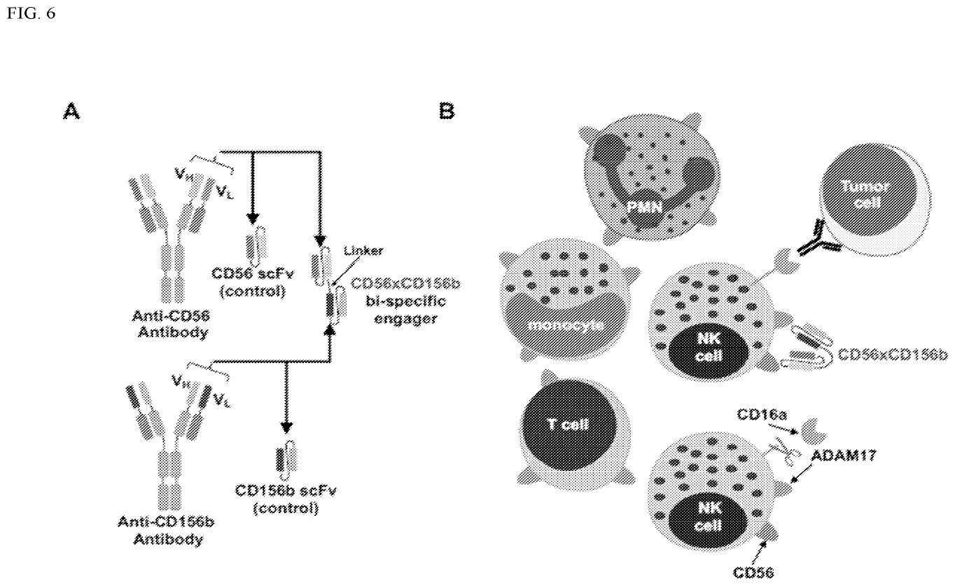

For example, a bi-specific antibody engager containing anti-ADAM17 domain and an anti-CD56 domain can be used to selectively block ADAM17 activity on natural killer (NK) cells. CD16 on NK cells is an Fc receptor that binds to antibodies attached to tumor cells. CD16 is cleaved by ADAM17. The cleaving of CD16 by ADAM17 causes downregulation of CD16 and decreases the efficacy of anti-tumor antibodies that rely on NK activity for therapeutic efficacy. Hence, ADAM17 is a regulatory checkpoint of CD16. An ADAM17×CD56 bi-specific engager (e.g., SEQ ID NO:37) could selectively block ADAM17 on NK cells to prevent CD16a cleavage and increase tumor cell killing (FIG. 6). Similarly, a TAB compound having a targeting domain having a different specificity could be used to block ADAM17 activity on T cells, monocytes, or other immune cell subsets. For example, an ADAM17×CD3 bi-specific engager can be used to selectively block ADAM17 activity on T cells. CD3 is a co-receptor involved in activating T cells, thus allowing specific targeting of T cells instead of NK cells.

Further, targeted ADAM17-blocking compounds can be used to target ADAM17 blockade to different tumors by using a targeting domain (e.g., an scFv or sdAb antibody fragment) that targets different tumor-associated antigens. Certain ADAM17 inhibitors (e.g., MEDI3622; MedImmune LLC, Gaithersburg, MD) recognize ADAM17 across species and blocks ADAM17 activity in other animal species, such as in dogs. Thus, biologic constructs containing one or more complementarity-determining region (CDR) from a cross-species ADAM17 inhibitor could function as an immunotherapeutic for more than one species. This is of particular importance considering immunotherapies, such as for cancer, are being rapidly developed for companion animals as well.

In one or more embodiments, the TAB compound can include more than one targeting domain. The presence of more than one targeting domain can increase the specificity of the TAB compound by requiring the TAB compound to bind to two markers on a target cell. For example, a TAB compound can include a broad anti-tumor targeting domain (e.g., an anti-Her2 antibody) and a second anti-tumor targeting domain (e.g., an anti-CD133 antibody or an anti-CD123 antibody) that is more specific to the targeted cell type.

A linker operably links the ADAM17-inhibiting domain and the targeting domain (or domains). As used herein, the term “operably linked” and variations thereof refer to direct or indirect covalent linking. Thus, two domains that are operably linked may be directly covalently coupled to one another. Conversely, the two operably linked domains may be connected by mutual covalent linking to an intervening moiety (e.g., a linker). Two domains may be considered operably linked if, for example, they are separated by the third domain. Exemplary linkers include amino acid sequences such as, for example, linkers having the amino acid sequence of any one of SEQ ID NO:26 through SEQ ID NO:35.

The ADAM17-inhibiting domain can include any moiety that interferes with ADAM17 activity against an ADAM17 substrate (e.g., CD16), or any other ADAM17 substrates. Exemplary ADAM17 substrates include tumor antigens, receptors involved in migration, etc. Exemplary moieties that are suitable as for use in the ADAM17-inhibiting domain include an anti-ADAM17 antibody, metalloproteinase inhibitor 3 (TIMPs), peptide, or a small molecule that binds to ADAM17 and inhibits ADAM17 activity against an ADAM17 substrate (e.g., TAPI-1, MilliporeSigma, Burlington, MA). Exemplary ADAM17 inhibitors include, but are not limited to, antibody fragments that include one or more of the complementarity-determining regions (CDRs) reflected in any one of SEQ ID NO: 13, SEQ ID NO:14, SEQ ID NO:15, SEQ ID NO:17, SEQ ID NO:18, or SEQ ID NO:19. Additional exemplary ADAM17 inhibitors include, but are not limited to, antibody fragments that include an amino acid sequence having up to 90% amino acid sequence similarity, as defined below, to any one of SEQ ID NO: 13, SEQ ID NO:14, SEQ ID NO:15, SEQ ID NO:17, SEQ ID NO:18, or SEQ ID NO:19. Still other exemplary ADAM17 inhibitors include, but are not limited to, antibody fragments that include an amino acid sequence having up to 90% amino acid sequence identity, as defined below, to any one of SEQ ID NO: 13, SEQ ID NO:14, SEQ ID NO:15, SEQ ID NO:17, SEQ ID NO:18, or SEQ ID NO:19. Such antibody fragments include, but are not limited to, antibody fragments having the amino acid sequences of any one of SEQ ID NO:12, SEQ ID NO:16, SEQ ID NO:20, SEQ ID NO:21, SEQ ID NO:22, SEQ ID NO:23, SEQ ID NO:24, SEQ ID NO:25, SEQ ID NO:36, SEQ ID NO:37, SEQ ID NO:38, SEQ ID NO:39, SEQ ID NO:40, SEQ ID NO:41, SEQ ID NO:42, SEQ ID NO:43, or SEQ ID NO:44.

Thus, a TAB compound can include, or be structurally similar to, the amino acid sequence of any one of SEQ ID NOs:36-44. The TAB compounds described herein provide the foundation for the design and construction of alternative TAB compounds. Thus, the modular nature of TAB compounds allows one to design a TAB compound using any desired ADAM17-inhibiting domain, any desired linker, and any desired targeting domain. Accordingly, a TAB compound can include, or be structurally similar to, a TAB compound designed to include any combination of a suitable ADAM17-inhibiting domain, a suitable linker, and a suitable targeting domain.

As used herein, a TAB compound is “structurally similar” to a reference TAB compound if the amino acid sequence of the candidate TAB compound possesses a specified amount of identity compared to the reference TAB compound. Structural similarity of two TAB compounds can be determined by aligning the residues of the two TAB compounds (for example, a candidate TAB compound and the TAB compound of, for example, any one of SEQ ID NOs:36-44) to optimize the number of identical amino acids along the lengths of their sequences; gaps in either or both sequences are permitted in making the alignment in order to optimize the number of identical amino acids, although the amino acids in each sequence must nonetheless remain in their proper order. A candidate TAB compound is the TAB compound being compared to the reference TAB compound (e.g., any one of SEQ ID NOs:36-44).

A pair-wise comparison analysis of amino acid sequences can be carried out using the BESTFIT algorithm in the GCG package (version 10.2, Madison WI). Alternatively, polypeptides may be compared using the Blastp program of the BLAST 2 search algorithm, as described by Tatiana et al., (FEMS Microbiol Lett, 174, 247-250 (1999)), and available on the National Center for Biotechnology Information (NCBI) website. The default values for all BLAST 2 search parameters may be used, including matrix=BLOSUM62; open gap penalty=11, extension gap penalty=1, gap x_dropoff=50, expect=10, wordsize=3, and filter on.

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. A conservative substitution for an amino acid may be selected from other members of the class to which the amino acid belongs. For example, it is well-known in the art of protein biochemistry that an amino acid belonging to a grouping of amino acids having a particular size or characteristic (such as charge, hydrophobicity, and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine, and glutamine. The positively charged (basic) amino acids include arginine, lysine, and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free —OH is maintained; and Gln for Asn to maintain a free —NH₂. Likewise, biologically active analogs of a polypeptide containing deletions or additions of one or more contiguous or noncontiguous amino acids that do not eliminate a functional activity of the polypeptide are also contemplated.

A TAB compound as described herein can include a TAB compound with at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence similarity to the reference amino acid sequence.

A TAB compound as described herein can include a TAB compound with at least 50%, at least 55%, at least 60%, at least 65%, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the reference amino acid sequence.

A TAB compound as described herein also can be designed to provide additional sequences, such as, for example, the addition of added C-terminal or N-terminal amino acids that can, for example, facilitate purification by trapping on columns or use of antibodies. Such tags include, for example, histidine-rich tags that allow purification of polypeptides on nickel columns. Such gene modification techniques and suitable additional sequences are well known in the molecular biology arts.

This disclosure also describes polynucleotides that encode a TAB compound. Exemplary polynucleotides that encode a TAB compound include polynucleotides that include the nucleotide sequence of any one of SEQ ID NOs:45-52 or otherwise encode any one of the TAB compounds having an amino acid sequence of any one of SEQ ID NOs:36-44. Since the genetic code is well-known, this disclosure describes any polynucleotide that encodes any embodiment of a TAB compound as described herein.

In another aspect, this disclosure describes a host cell including any of the isolated nucleic acid sequences and/or proteins described herein. Thus, this disclosure encompasses translation of a nucleic acid (e.g., an mRNA) by a host cell to produce a TAB compound.

The nucleic acid constructs of the present invention may be introduced into a host cell to be altered, thus allowing expression of the TAB compound within the cell, thereby generating a genetically engineered cell. A variety of methods are known in the art and suitable for introduction of nucleic acid into a cell, including viral and non-viral mediated techniques. Examples of typical non-viral mediated techniques include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion. Other methods of transfection include proprietary transfection reagents such as LIPOFECTAMINE (Thermo Fisher Scientific, Inc., Waltham, MA), HILYMAX (Dojindo Molecular Technologies, Inc., Rockville, MD), FUGENE (Promega Corp., Madison, WI), JETPEI (Polyplus Transfection, Illkirch, France), EFFECTENE (Qiagen, Hilden, Germany) and DreamFect (OZ Biosciences, Inc USA, San Diego, CA).

The nucleic acid constructs described herein may be introduced into a host cell to be altered, thus allowing expression within the cell of the protein encoded by the nucleic acid. A variety of host cells are known in the art and suitable for protein expression. Examples of typical cell used for transfection and protein expression include, but are not limited to, a bacterial cell, a eukaryotic cell, a yeast cell, an insect cell, or a plant cell such as, for example, E. coli, Bacillus, Streptomyces, Pichia pastoris, Salmonella typhimurium, Drosophila S2, Spodoptera SJ9, CHO, COS (e.g., COS-7), 3T3-F442A, HeLa, HUVEC, HUAEC, NIH 3T3, Jurkat, 293, 293H, or 293F.

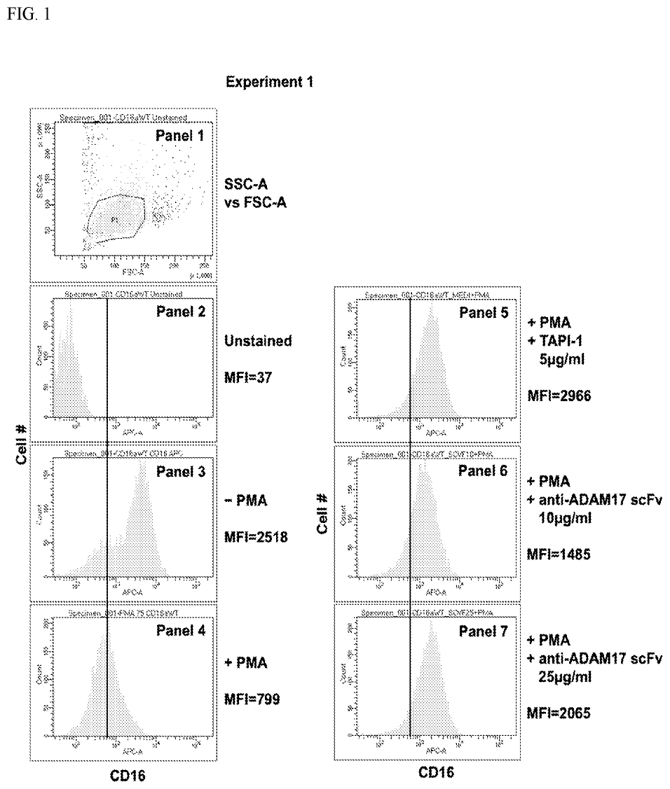

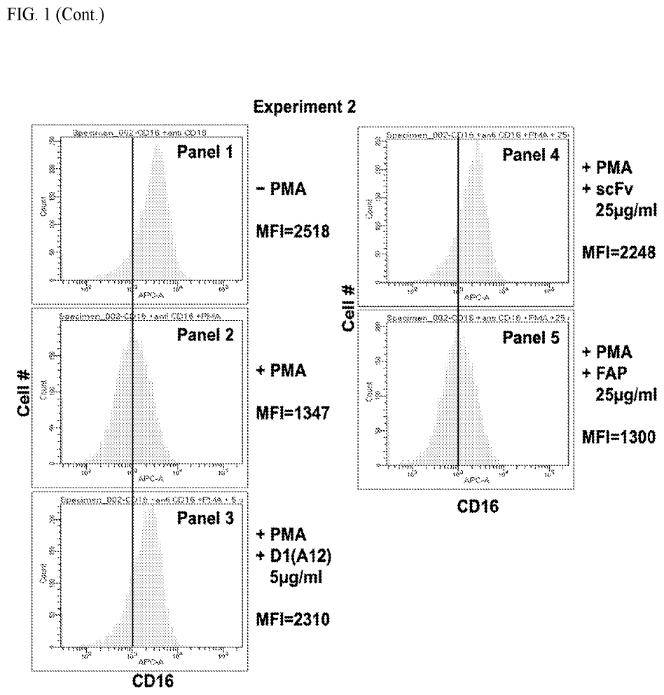

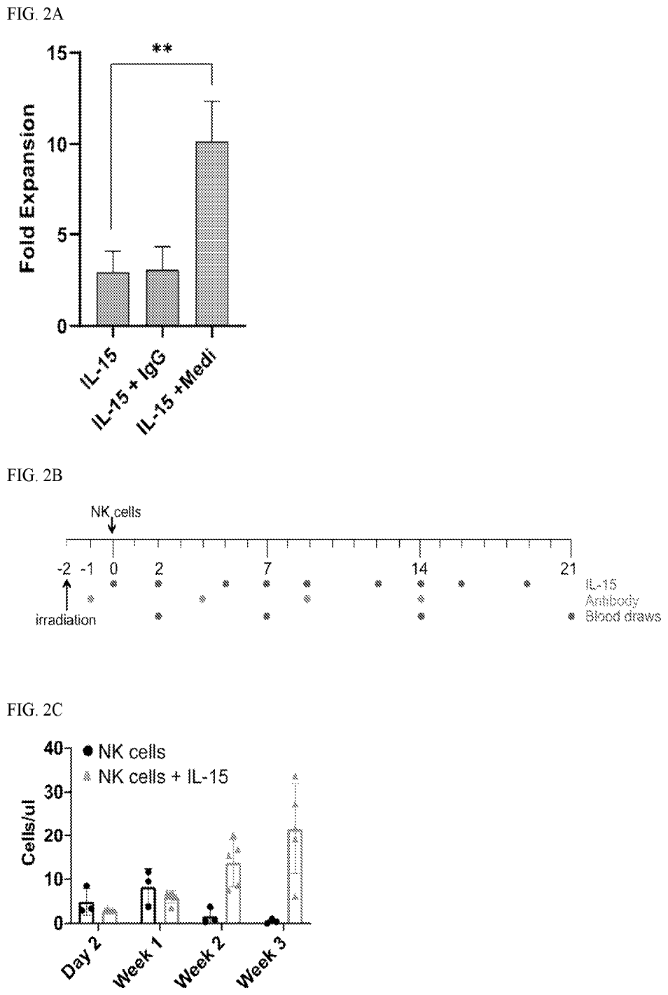

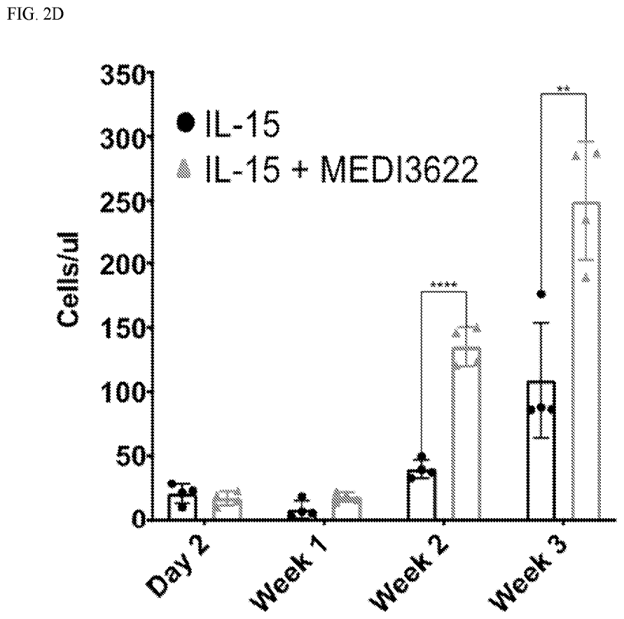

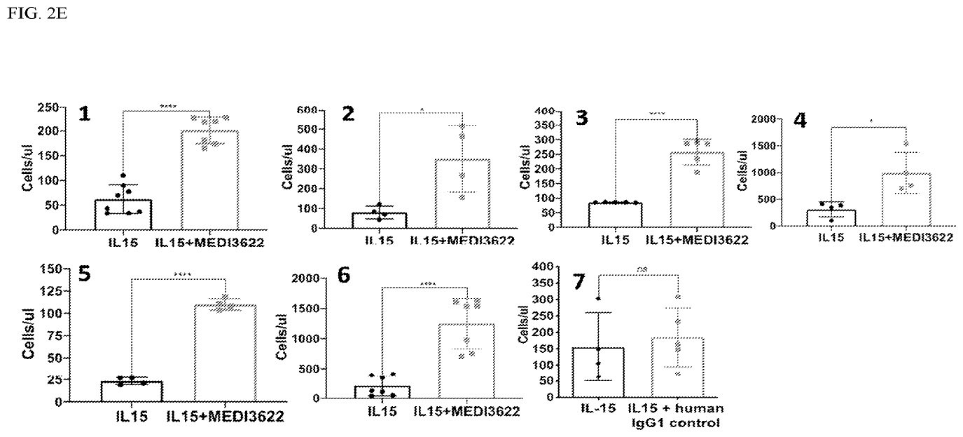

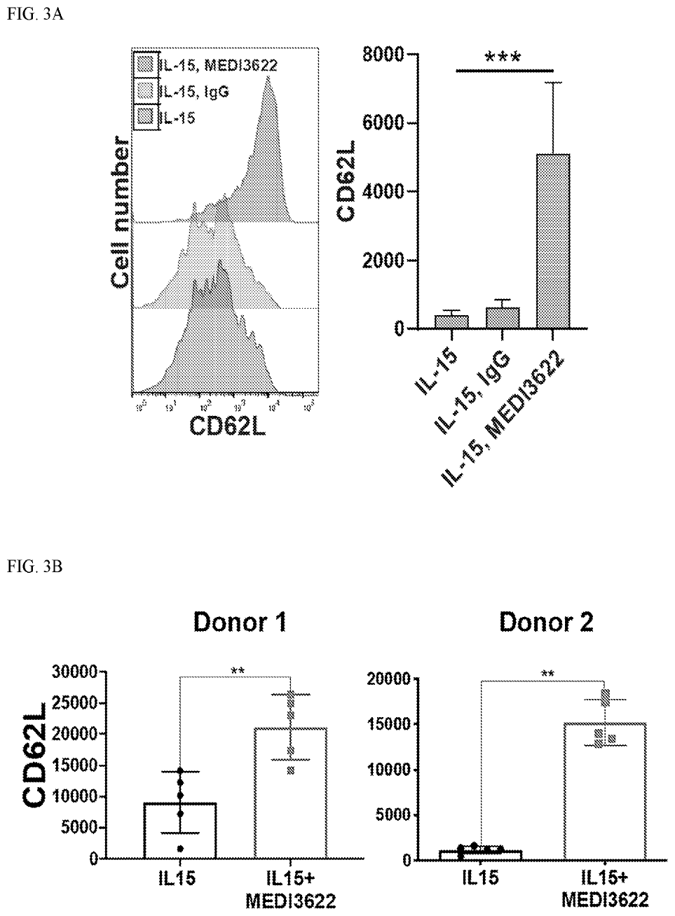

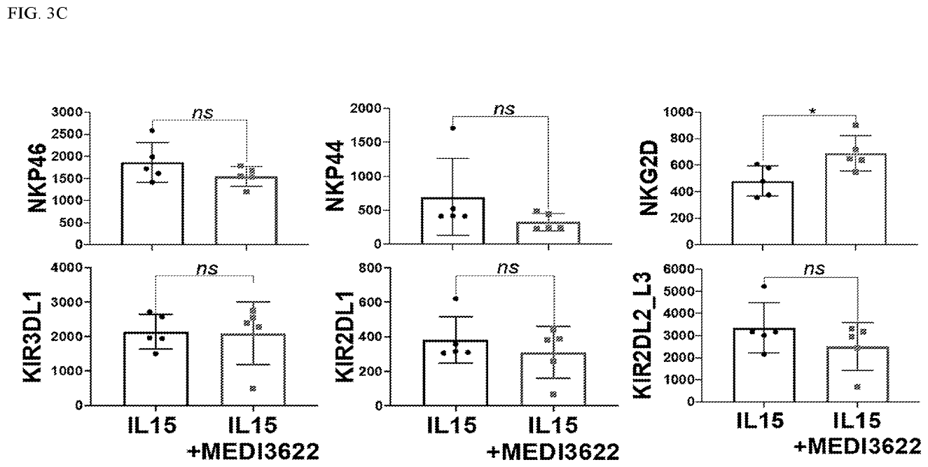

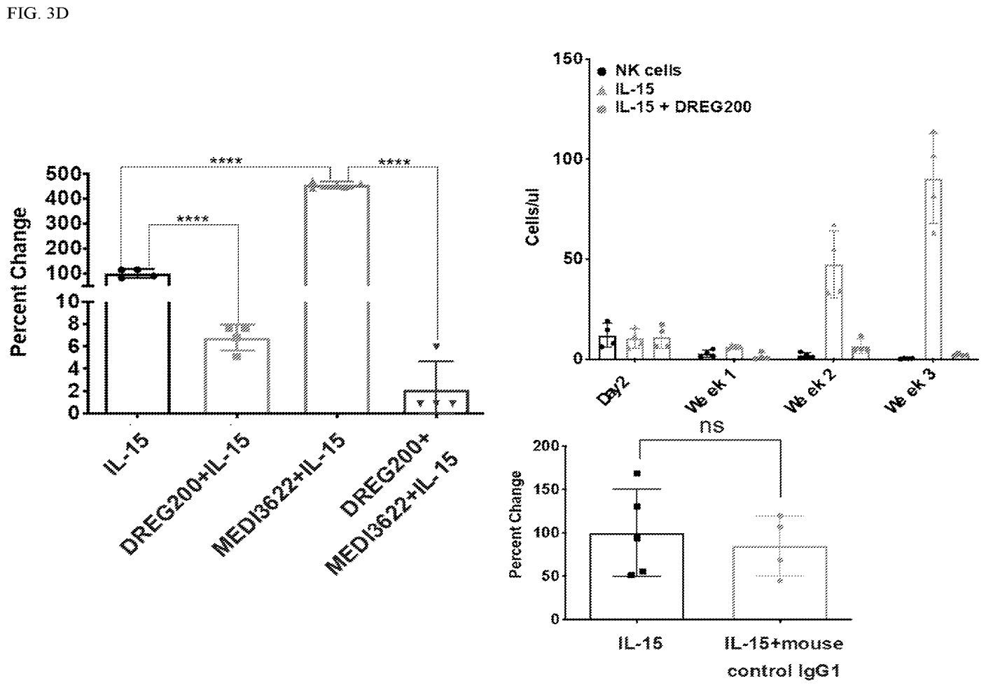

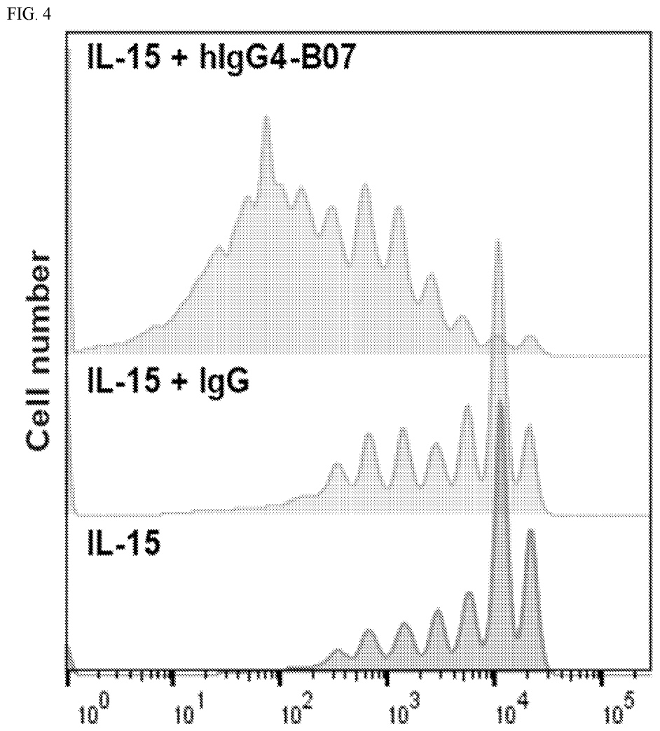

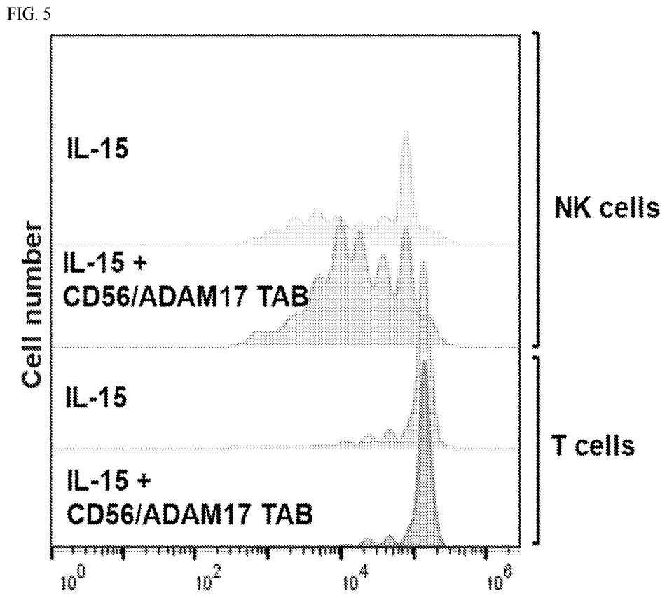

In another aspect, this disclosure describes using a TAB compound to increase proliferation of Natural Killer (NK) cells. Generally, the method includes contacting a population of NK cells with the TAB compound under conditions effective for the TAB compound to induce proliferation of the NK cells. FIGS. 1-4 provide data showing that blocking ADAM17 enhances NK cell proliferation. FIG. 5 shows provides data showing that a TAB compound that includes an ADAM-17-inhibiting domain retains ADAM17 inhibitory activity and increases proliferation of NK cells. Moreover, FIG. 5 shows that the proliferation of NK cells is specific; similarly treated T cells do not proliferate. Thus, a TAB compound designed to selectively target NK cells may be used to selectively enhance proliferation of NK cells.

In another aspect, this disclosure describes methods of killing a target cell in a subject. Generally, the method includes administering to the subject a TAB compound in an amount effective to reduce ADAM17-mediated function.

In yet another aspect, this disclosure describes methods for treating a subject having, or at risk of having, a medical condition treatable by inhibiting ADAM17 activity. Exemplary conditions treatable by inhibiting ADAM17 activity include, but are not limited to, cancers and other tumor-associated conditions, infectious conditions, and inflammatory diseases. “Treat” or variations thereof refer to reducing, limiting progression, ameliorating, or resolving, to any extent, the symptoms or signs related to a condition. As used herein, “ameliorate” refers to any reduction in the extent, severity, frequency, and/or likelihood of a symptom or clinical sign characteristic of a particular condition; “symptom” refers to any subjective evidence of disease or of a patient's condition; and “sign” or “clinical sign” refers to an objective physical finding relating to a particular condition capable of being found by one other than the patient.

The subject can be a human or a non-human animal such as, for example, a livestock animal or a companion animal. Exemplary non-human animal subjects include, but are not limited to, animals that are hominid (including, for example chimpanzees, gorillas, and orangutans), bovine (including, for instance, cattle), caprine (including, for instance, goats), ovine (including, for instance, sheep), porcine (including, for instance, swine), equine (including, for instance, horses), members of the family Cervidae (including, for instance, deer, elk, moose, caribou and reindeer), members of the family Bison (including, for instance, bison), feline (including, for example, tigers, lions, and domesticated cats), canine (including, for example, a wolves and domesticated dogs), avian (including, for example, turkeys, chickens, ducks, and geese), a rodent (including, for example, mice or rats), a member of the family Leporidae (including, for example, rabbits or hares), members of the family (Mustelidae (including, for example ferrets), or member of the order Chiroptera (including, for example, bats).

A “treatment” may be therapeutic or prophylactic. “Therapeutic” and variations thereof refer to a treatment that ameliorates one or more existing symptoms or clinical signs associated with a condition. “Prophylactic” and variations thereof refer to a treatment that limits, to any extent, the development and/or appearance of a symptom or clinical sign of a condition. Generally, a “therapeutic” treatment is initiated after the condition manifests in a subject, while “prophylactic” treatment is initiated before a condition manifests in a subject. Thus, in certain embodiments, the method can involve prophylactic treatment of a subject at risk of developing a condition. “At risk” refers to a subject that may or may not actually possess the described risk.

Thus, for example, a subject “at risk” of infectious condition is a subject present in an area where other individuals have been identified as having the infectious condition and/or is likely to be exposed to the infectious agent even if the subject has not yet manifested any detectable indication of infection by the pathogen (e.g., a virus, a bacterium, a fungus, or a parasite) and regardless of whether the subject may harbor a subclinical amount of the pathogen. As another example, a subject “at risk” of a non-infectious condition is a subject possessing one or more risk factors associated with the condition such as, for example, genetic predisposition, ancestry, age, sex, geographical location, lifestyle, or medical history. Treatment may also be continued after symptoms have resolved, for example to prevent or delay their recurrence.

Accordingly, a TAB compound may be administered before, during, or after the subject first exhibits a symptom or clinical sign of the condition. Treatment initiated before the subject first exhibits a symptom or clinical sign associated with the condition may result in decreasing the likelihood that the subject experiences clinical evidence of the condition compared to a subject to which the TAB compound is not administered, decreasing the severity of symptoms and/or clinical signs of the condition, and/or completely resolving the condition. Treatment initiated after the subject first exhibits a symptom or clinical sign associated with the condition may result in decreasing the severity of symptoms and/or clinical signs of the condition compared to a subject to which the composition is not administered, and/or completely resolving the condition.

The TAB compound can be any embodiment of the TAB compound described above having a targeting domain that selectively binds to an appropriate target cell population. In some cases, the target can be a tumor cell so that the method can involve treating cancer associated with the tumor cells. Thus, In one or more embodiments, the method can include ameliorating at least one symptom or clinical sign of the tumor. In other cases, the target can be a cell infected by a pathogen (e.g., a bacterium, a virus, a fungus, or a parasite) so that the method can involve treating a condition caused by such an infection by the pathogen. Thus, In one or more embodiments, the method can include ameliorating at least one symptom or clinical sign of an infection.

In embodiments in which the target cell includes a tumor cell, the method can further include surgically resecting the tumor and/or reducing the size of the tumor through chemical (e.g., chemotherapeutic) and/or radiation therapy. Exemplary tumors that may be treated include tumors associated with prostate cancer, lung cancer, colon cancer, rectum cancer, urinary bladder cancer, melanoma, kidney cancer, renal cancer, oral cavity cancer, pharynx cancer, pancreas cancer, uterine cancer, thyroid cancer, skin cancer, head and neck cancer, cervical cancer, ovarian cancer and/or hematopoietic cancer.

A TAB compound described herein may be formulated with a pharmaceutically acceptable carrier. As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with a TAB compound without causing any undesirable biological effects or interacting in a deleterious manner with any of the other components of the pharmaceutical composition in which it is contained.

A TAB compound may therefore be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A composition also can be administered via a sustained or delayed release.

Thus, a TAB compound may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

A formulation may be conveniently presented in unit dosage form and may be prepared by methods well known in the art of pharmacy. Methods of preparing a composition with a pharmaceutically acceptable carrier include the step of bringing a TAB compound into association with a carrier that constitutes one or more accessory ingredients. In general, a formulation may be prepared by uniformly and/or intimately bringing the active molecule into association with a liquid carrier, a finely divided solid carrier, or both, and then, if necessary, shaping the product into the desired formulations.

The amount of TAB compound administered can vary depending on various factors including, but not limited to, the specific TAB compound being used, the weight, physical condition, and/or age of the subject, and/or the route of administration. Thus, the absolute weight of TAB compound included in a given unit dosage form can vary widely, and depends upon factors such as the species, age, weight, and physical condition of the subject, and/or the method of administration. Accordingly, it is not practical to set forth generally the amount that constitutes an amount of TAB compound effective for all possible applications. Those of ordinary skill in the art, however, can readily determine the appropriate amount with due consideration of such factors.

In one or more embodiments, the method can include administering sufficient TAB compound to provide a dose of, for example, from about 100 ng/kg to about 50 mg/kg to the subject, although In one or more embodiments the methods may be performed by administering TAB compound in a dose outside this range.

In one or more embodiments, the method can includes administering sufficient TAB compound to provide a minimum dose of at least 100 ng/kg such as, for example, at least 1 μg/kg, at least 5 μg/kg, at least 10 μg/kg, at least 25 μg/kg, at least 50 μg/kg, at least 100 μg/kg, at least 200 μg/kg, at least 300 μg/kg, at least 400 μg/kg, at least 500 μg/kg, at least 600 μg/kg, at least 700 μg/kg, at least 800 μg/kg, at least 900 μg/kg, or at least 1 mg/kg.

In one or more embodiments, the method includes administering sufficient TAB compound to provide a maximum dose of no more than 10 mg/kg such as, for example, no more than 5 mg/kg, no more than 4 mg/kg, no more than 3 mg/kg, no more than 2 mg/kg, no more than 1 mg/kg, no more than 900 μg/kg, no more than 800 μg/kg, no more than 700 μg/kg, no more than 600 μg/kg, no more than 500 μg/kg, no more than 400 μg/kg, no more than 300 μg/kg, no more than 200 μg/kg, no more than 100 μg/kg, no more than 90 μg/kg, no more than 80 g/kg, no more than 70 μg/kg, no more than 60 μg/kg, no more than 50 μg/kg, no more than 40 g/kg, no more than 30 μg/kg, no more than 20 μg/kg, or no more than 10 μg/kg/day. TAB compound

In one or more embodiments, the method includes administering sufficient TAB compound to provide a dose characterized by a range having endpoints defined by any a minimum dose identified above and any maximum dose that is greater than the selected minimum dose. For example, In one or more embodiments, the method can include administering sufficient TAB compound to provide a dose of from about 10 μg/kg to about 10 mg/kg to the subject, a dose of from about 100 μg/kg to about 1 mg/kg, a dose of from 5 μg/kg to 100 μg/kg, etc.

In one or more embodiments, a TAB compound may be administered, for example, from a single dose to multiple doses per week, although In one or more embodiments the method can be performed by administering a TAB compound at a frequency outside this range. In certain embodiments, a TAB compound may be administered from about once per month to about five times per week. In one or more embodiments, a dose may be described in terms of the amount of TAB compound administered over a 24-hour period, which may be administered in a seven-day cycle of four days of, for example, treatment and three days of rest.

In one or more embodiments, a TAB compound may be administered, for example, from a single dose to multiple cycles of treatment, although In one or more embodiments the method can be performed by administering a TAB compound for a duration outside this range. In one or more embodiments, the TAB compound may be administered for three weeks. In such embodiments, each week may be a treatment cycle such as the exemplary treatment cycle described in the preceding paragraph. In other embodiments, the TAB compound may be administered for a greater number of treatment cycles, without a gap between one set of treatment cycles and a subsequent set of treatment cycles. The gap between one set of treatment cycles and a subsequent set of treatment cycles may be a gap of one or more weeks, one or more months, or one or more years.

In one or more embodiments, the method further includes administering one or more additional therapeutic agents. The one or more additional therapeutic agents may be administered before, after, and/or coincident to the administration of a TAB compound. A TAB compound and the additional therapeutic agents may be co-administered. As used herein, “co-administered” refers to two or more components of a combination administered so that the therapeutic or prophylactic effects of the combination can be greater than the therapeutic or prophylactic effects of either component administered alone. Two components may be co-administered simultaneously or sequentially. Simultaneously co-administered components may be provided in one or more pharmaceutical compositions. Sequential co-administration of two or more components includes cases in which the components are administered so that each component can be present at the treatment site at the same time. Alternatively, sequential co-administration of two components can include cases in which at least one component has been cleared from a treatment site, but at least one cellular effect of administering the component (e.g., cytokine production, activation of a certain cell population, etc.) persists at the treatment site until one or more additional components are administered to the treatment site. Thus, a co-administered combination can, in certain circumstances, include components that never exist in a chemical mixture with one another. In other embodiments, the TAB compound and the additional therapeutic agent may be administered as part of a mixture or cocktail. In some aspects, the administration of TAB compound may allow for the effectiveness of a lower dosage of other therapeutic modalities when compared to the administration of the other therapeutic agent or agents alone, thereby decreasing the likelihood, severity, and/or extent of the toxicity observed when a higher dose of the other therapeutic agent or agents is administered.

Exemplary additional therapeutic agents include an anti-tumor chemotherapeutic agent such as, for example, altretamine, amsacrine, L-asparaginase, colaspase, bleomycin, busulfan, capecitabine, carboplatin, carmustine, chlorambucil, cisplatin, cladribine, cyclophosphamide, cytophosphane, cytarabine, dacarbazine, dactinomycin, daunorubicin, docetaxel, doxorubicin, epirubicin, etoposide, fluorouracil, fludarabine, fotemustine, ganciclovir, gemcitabine, hydroxyurea, idarubicin, ifosfamide, irinotecan, lomustine, melphalan, mercaptopurine, methotrexate, mitoxantrone, mitomycin C, nimustine, oxaliplatin, paclitaxel, pemetrexed, procarbazine, raltitrexed, temozolomide, teniposide, tioguanine, thiotepa, topotecan, vinblastine, vincristine, vindesine, and vinorelbine.

In one or more embodiments, administering the TAB compound and at least one additional therapeutic agent produces therapeutic synergy. In some aspects of the methods of the present invention, a measurement of the response to treatment observed after administering both a TAB compound as described herein and the additional therapeutic agent is improved over the same measurement of response to treatment observed after administering either the TAB compound or the additional therapeutic agent alone.

### Anti-ADAM17 Antibodies

In another aspect, this disclosure describes a canine IgG4 anti-ADAM17 antibody that selectively binds to ADAM17 and a human IgG4 anti-ADAM17 antibody that selectively binds to ADAM17 to antagonize its function. The anti-ADAM17 antibodies were engineered to modulate their IgG properties. The anti-ADAM17 antibodies are of an IgG4 isotype to reduce immune-related effector functions, including the induction of antibody-dependent cell-mediated cytotoxicity (ADCC) and complement-dependent cytotoxicity (CDC). The human anti-ADAM17 antibody was engineered with a serine-228 to proline mutation (S228P) to increase antibody stability. Hence, the human anti-ADAM17 antagonizing antibody has been optimized for reduced effector function and increased half-life (IgG4, S228P).

As used herein, the term “selectively binds” refers to the ability to differentiate between two or more alternatives such as, for example, having differential affinity, to any degree, for a particular target. For brevity, reference to an antibody that selectively binds to a specified target (e.g., ADAM17) includes any antibody fragment that exhibits the described binding character. Similarly, reference to a ligand that selectively binds to a specified target includes any fragment of the ligand that exhibits the described binding character. For example, a canine IgG4 anti-ADAM17 antibody or a human IgG4 anti-ADAM17 antibody can include any antibody fragment that includes one or more of the CDRs reflected in any one of SEQ ID NO:13, SEQ ID NO:14, SEQ ID NO:15, SEQ ID NO:17, SEQ ID NO:18, or SEQ ID NO:19.

A human IgG4 anti-ADAM17 antibody or a canine IgG4 anti-ADAM17 antibody (collective, hereafter, “IgG4 anti-ADAM17 antibody) as described herein can be incorporated into biologic constructs that may be used in the context of, for example, therapeutic, diagnostic, and/or detection methods. For example, an IgG4 anti-ADAM17 antibody or a portion thereof (e.g., one or more CDRs) described herein may be incorporated into Targeted ADAM17 Blocker (TAB) compound, a bi-specific Killer engager molecule (BiKE), a tri-specific Killer engager molecule, a tetra-specific Killer engager molecule, a penta-specific Killer engager molecule, a bi-specific T cell engager molecule, a tri-specific T cell engager molecule, a tetra-specific T cell engager molecule, a penta-specific T cell engager molecule, a chimeric antigen receptor (CAR, for expression in, for example, CAR T cells, CAR NK cells, CAR macrophage cells, etc.), a full antibody construct (e.g., to induce antibody-dependent cellular toxicity (ADCC)), an antibody-drug conjugate (ADC) molecule (e.g., for delivery of a toxin), a labeling construct (e.g., for commercial evaluation of antigen expression), a radiolabeled format (e.g., for positron-emission tomography (PET) imaging or directed radiation delivery), an applications for delivery of a cytokine and/or a chemokine, or other general immunotherapeutic approach.

Exemplary human IgG4 anti-ADAM17 antibody fragments are reflected in the amino acid sequences of SEQ ID NO:20 and SEQ ID NO:21. Exemplary canine IgG4 anti-ADAM17 antibody fragments are reflected in the amino acid sequences of SEQ ID NO:22 and SEQ ID NO:23. Additional IgG4 anti-ADAM17 antibody fragments include polypeptides that are structurally similar to, or are functional variants of, the amino acid sequence of SEQ ID NO:20, SEQ ID NO:21, SEQ ID NO:22, or SEQ ID NO:23. As used herein, an IgG4 anti-ADAM17 antibody fragment is “structurally similar” to or a “functional variant” of SEQ ID NO:20, SEQ ID NO:21, SEQ ID NO:22, or SEQ ID NO:23 if the amino acid sequence of the IgG4 anti-ADAM17 antibody fragment possesses a specified amount of identity compared to the reference polypeptide. An amino acid sequence is a “functional fragment” of a reference amino acid sequence if the “functional fragment” amino acid sequence contains less than the full-length amino acid sequence of the reference amino acid sequence. A “functional fragment” may further possess a specified amount of sequence identity or sequence specificity compared to the reference amino acid sequence.

Structural similarity and/or sequence identity of two polypeptides can be determined by aligning the amino acid residues of the two polypeptides (for example, a candidate IgG4 anti-ADAM17 antibody fragment and the polypeptide of, for example, SEQ ID NO:20, SEQ ID NO:21, SEQ ID NO:22, or SEQ ID NO:23) to optimize the number of identical amino acids along the lengths of their sequences. Gaps in either or both sequences are permitted in making the alignment to optimize the number of identical amino acids, although the amino acids in each sequence must nonetheless remain in their proper order. A candidate IgG4 anti-ADAM17 antibody is the polypeptide being compared to the reference polypeptide (e.g., SEQ ID NO:20, SEQ ID NO:21, SEQ ID NO:22, or SEQ ID NO:23).

A pair-wise comparison analysis of amino acid sequences can be carried out using the BESTFIT algorithm in the GCG package (version 10.2, Madison WI). Alternatively, polypeptides may be compared using the Blastp program of the BLAST 2 search algorithm, as described by Tatiana et al., (FEMS Microbiol Lett, 174, 247-250 (1999)), and available on the National Center for Biotechnology Information (NCBI) website. The default values for all BLAST 2 search parameters may be used, including matrix=BLOSUM62; open gap penalty=11, extension gap penalty=1, gap x_dropoff=50, expect=10, wordsize=3, and filter on.

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. A conservative substitution for an amino acid in an IgG4 anti-ADAM17 antibody may be selected from other members of the class to which the amino acid belongs. For example, it is well-known in the art of protein biochemistry that an amino acid belonging to a grouping of amino acids having a particular size or characteristic (such as charge, hydrophobicity, and hydrophilicity) can be substituted for another amino acid without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine, and glutamine. The positively charged (basic) amino acids include arginine, lysine, and histidine. The negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free —OH is maintained; and Gln for Asn to maintain a free —NH₂. Likewise, biologically active analogs of a polypeptide containing deletions or additions of one or more contiguous or noncontiguous amino acids that do not eliminate a functional activity of the polypeptide are also contemplated.

In one or more embodiments, an IgG4 anti-ADAM17 antibody can include a polypeptide with at least 90%, at least 91%, at least 92%, at least 93%, at least 94%, at least 95%, at least 96%, at least 97%, at least 98%, or at least 99% amino acid sequence similarity to the reference amino acid sequence.

In one or more embodiments, an IgG4 anti-ADAM17 antibody can include at least 90%, at least 91%, at least 92%, at least 93%, at least 94%, at least 95%, at least 96%, at least 97%, at least 98%, or at least 99% amino acid sequence identity to the reference amino acid sequence.

In one or more embodiments, an IgG4 anti-ADAM17 antibody also can be designed to provide additional sequences, such as, for example, amino acids added C-terminal or N-terminal of the human IgG4 anti-ADAM17 antibody. Such additional amino acids may include, for example, a signal sequence or a tag that facilitates purification by trapping the tagged IgG4 anti-ADAM17 antibody on a column or by using antibodies. Exemplary tags include, for example, a histidine-rich tag that allows purification of polypeptides on nickel columns.

This disclosure also describes polynucleotides that encode an IgG4 anti-ADAM17 antibody fragment. Exemplary polynucleotides that encode an IgG4 anti-ADAM17 antibody fragment include polynucleotides that encode the polypeptide sequence of SEQ ID NO:20, SEQ ID NO:21, SEQ ID NO:22, or SEQ ID NO:23. Since the genetic code is well-known, this disclosure describes any polynucleotide that encodes an IgG4 anti-ADAM17 antibody as described herein.

This disclosure further describes polypeptides that include an antibody fragment that includes the CDRs reflected in one or more of SEQ ID NO:13, SEQ ID NO:14, SEQ ID NO:15, SEQ ID NO:17, SEQ ID NO:18, or SEQ ID NO:19, regardless of the isotype or subclass of antibody. Thus, the antibody fragment can be a fragment of an IgG1 antibody, an IgG2 antibody, an IgG3 antibody, an IgG4 antibody, an IgA antibody, an IgE antibody, an IgD antibody, or an IgM antibody.

This disclosure also describes polynucleotides that encode a polypeptide that includes an antibody fragment that includes the CDRs reflected in one or more of SEQ ID NO:13, SEQ ID NO:14, SEQ ID NO:15, SEQ ID NO:17, SEQ ID NO:18, or SEQ ID NO:19. As noted above, since the genetic code is well-known, this disclosure describes any polynucleotide that encodes any polypeptide described herein.

In another aspect, this disclosure describes a host cell including any of the isolated nucleic acid sequences and/or proteins described herein. Thus, this disclosure encompasses translation of a nucleic acid (e.g., an mRNA) by a host cell to produce an anti-ADAM17 antibody.

The nucleic acid constructs of the present invention may be introduced into a host cell to be altered, thus allowing expression of the anti-ADAM17 antibody within the cell, thereby generating a genetically engineered cell. A variety of methods are known in the art and suitable for introduction of nucleic acid into a cell, including viral and non-viral mediated techniques. Examples of typical non-viral mediated techniques include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion. Other methods of transfection include proprietary transfection reagents such as LIPOFECTAMINE (Thermo Fisher Scientific, Inc., Waltham, MA), HILYMAX (Dojindo Molecular Technologies, Inc., Rockville, MD), FUGENE (Promega Corp., Madison, WI), JETPEI (Polyplus Transfection, Illkirch, France), EFFECTENE (Qiagen, Hilden, Germany) and DreamFect (OZ Biosciences, Inc USA, San Diego, CA).

The nucleic acid constructs described herein may be introduced into a host cell to be altered, thus allowing expression within the cell of the protein encoded by the nucleic acid. A variety of host cells are known in the art and suitable for protein expression. Examples of typical cell used for transfection and protein expression include, but are not limited to, a bacterial cell, a eukaryotic cell, a yeast cell, an insect cell, or a plant cell such as, for example, E. coli, Bacillus, Streptomyces, Pichia pastoris, Salmonella typhimurium, Drosophila S2, Spodoptera SJ9, CHO, COS (e.g., COS-7), 3T3-F442A, HeLa, HUVEC, HUAEC, NIH 3T3, Jurkat, 293, 293H, or 293F.

An IgG4 anti-ADAM17 antibody as described herein, whether human or canine, may be incorporated into a biologic, which is then formulated with a pharmaceutically acceptable carrier. As used herein, an “anti-ADAM17 biologic” is a biologic compound that includes an anti-ADAM17 polypeptide. An anti-ADAM17 biologic may include additional functional moieties depending on the fundamental structural platform of the biologic (e.g., a BiKE, a TriKE, a CAR, etc.).

For example, FIG. 5 provides data demonstrating biological activity of an ADAM17 biologic that includes human IgG4 anti-ADAM17 antibody as the anti-ADAM17 domain. FIG. 5 shows that an anti-ADAM17 biologic (CD56/ADAM17 TAB) possesses ADAM17 inhibitory activity and increases proliferation of NK cells. Moreover, FIG. 5 shows that the proliferation of NK cells is specific; similarly treated T cells do not proliferate. Thus, an anti-ADAM17 biologic may be used to selectively enhance proliferation of NK cells.

As used herein, “carrier” includes any solvent, dispersion medium, vehicle, coating, diluent, antibacterial, and/or antifungal agent, isotonic agent, absorption delaying agent, buffer, carrier solution, suspension, colloid, and the like. The use of such media and/or agents for pharmaceutical active substances is well known in the art. Except insofar as any conventional media or agent is incompatible with the active ingredient, its use in the therapeutic compositions is contemplated. Supplementary active ingredients also can be incorporated into the compositions. As used herein, “pharmaceutically acceptable” refers to a material that is not biologically or otherwise undesirable, i.e., the material may be administered to an individual along with the anti-ADAM17 biologic without causing any undesirable biological effects or interacting in a deleterious manner with any of the other components of the pharmaceutical composition in which it is contained.

An anti-ADAM17 biologic may therefore be formulated into a pharmaceutical composition. The pharmaceutical composition may be formulated in a variety of forms adapted to a preferred route of administration. Thus, a composition can be administered via known routes including, for example, oral, parenteral (e.g., intradermal, transcutaneous, subcutaneous, intramuscular, intravenous, intraperitoneal, etc.), or topical (e.g., intranasal, intrapulmonary, intramammary, intravaginal, intrauterine, intradermal, transcutaneous, rectally, etc.). A pharmaceutical composition can be administered to a mucosal surface, such as by administration to, for example, the nasal or respiratory mucosa (e.g., by spray or aerosol). A composition also can be administered via a sustained or delayed release.

Thus, an anti-ADAM17 biologic may be provided in any suitable form including but not limited to a solution, a suspension, an emulsion, a spray, an aerosol, or any form of mixture. The composition may be delivered in formulation with any pharmaceutically acceptable excipient, carrier, or vehicle. For example, the formulation may be delivered in a conventional topical dosage form such as, for example, a cream, an ointment, an aerosol formulation, a non-aerosol spray, a gel, a lotion, and the like. The formulation may further include one or more additives including such as, for example, an adjuvant, a skin penetration enhancer, a colorant, a fragrance, a flavoring, a moisturizer, a thickener, and the like.

A formulation may be conveniently presented in unit dosage form and may be prepared by methods well known in the art of pharmacy. Methods of preparing a composition with a pharmaceutically acceptable carrier include the step of bringing the anti-ADAM17 biologic into association with a carrier that constitutes one or more accessory ingredients. In general, a formulation may be prepared by uniformly and/or intimately bringing the active compound into association with a liquid carrier, a finely divided solid carrier, or both, and then, if necessary, shaping the product into the desired formulations.

Thus, in another aspect, this disclosure describes a method of treating any condition in which reducing ADAM17 activity has therapeutic benefit. Generally, the method includes administering to the subject an amount of the anti-ADAM17 biologic effective for treating the condition. “Treat” or variations thereof refer to reducing, limiting progression, ameliorating, or resolving, to any extent, the symptoms or signs related to a condition. As used herein, “ameliorate” refers to any reduction in the extent, severity, frequency, and/or likelihood of a symptom or clinical sign characteristic of a particular condition; “symptom” refers to any subjective evidence of disease or of a patient's condition; and “sign” or “clinical sign” refers to an objective physical finding relating to a particular condition capable of being found by one other than the patient.

The subject can be a human or a non-human animal such as, for example, a livestock animal or a companion animal. Exemplary non-human animal subjects include, but are not limited to, animals that are hominid (including, for example chimpanzees, gorillas, and orangutans), bovine (including, for instance, cattle), caprine (including, for instance, goats), ovine (including, for instance, sheep), porcine (including, for instance, swine), equine (including, for instance, horses), members of the family Cervidae (including, for instance, deer, elk, moose, caribou and reindeer), members of the family Bison (including, for instance, bison), feline (including, for example, tigers, lions, and domesticated cats), canine (including, for example, a wolves and domesticated dogs), avian (including, for example, turkeys, chickens, ducks, and geese), a rodent (including, for example, mice or rats), a member of the family Leporidae (including, for example, rabbits or hares), members of the family (Mustelidae (including, for example ferrets), or member of the order Chiroptera (including, for example, bats).

A “treatment” may be therapeutic or prophylactic. “Therapeutic” and variations thereof refer to a treatment that ameliorates one or more existing symptoms or clinical signs associated with a condition. “Prophylactic” and variations thereof refer to a treatment that limits, to any extent, the development and/or appearance of a symptom or clinical sign of a condition. Generally, a “therapeutic” treatment is initiated after the condition manifests in a subject, while “prophylactic” treatment is initiated before a condition manifests in a subject. Thus, in certain embodiments, the method can involve prophylactic treatment of a subject at risk of developing a condition. “At risk” refers to a subject that may or may not actually possess the described risk. Thus, for example, a subject “at risk” for developing a specified condition is a subject that possesses one or more indicia of increased risk of having, or developing, the specified condition compared to individuals who lack the one or more indicia, regardless of the whether the subject manifests any symptom or clinical sign of having or developing the condition. Exemplary indicia of a condition can include, for example, genetic predisposition, ancestry, age, sex, geographical location, lifestyle, or medical history. Treatment may also be continued after symptoms have resolved, for example to prevent or delay their recurrence.

In one or more embodiments, “prophylactic” treatment also includes treatment in the setting of relapse. In other words, a subject may have once manifested symptoms or clinical signs of a condition but received successful treatment so that the condition is considered to be in remission. Such as subject may no longer manifest a symptom or clinical sign of the condition in remission but be “at risk” of relapse. In such a context, treatment that includes the anti-ADAM17 biologic may be considered “prophylactic” to reduce the likelihood and/or severity of relapse.

Accordingly, an anti-ADAM17 biologic may be administered to the subject before, during, or after the subject first exhibits a symptom or clinical sign of the condition. Treatment initiated before the subject first exhibits a symptom or clinical sign associated with the condition may result in decreasing the likelihood that the subject experiences clinical evidence of the condition compared to a subject to which the anti-ADAM17 biologic is not administered, decreasing the severity of symptoms and/or clinical signs of the condition, and/or completely resolving the condition. Treatment initiated before the subject first exhibits a symptom or clinical sign associated with relapse of the condition may result in decreasing the likelihood that the subject experiences clinical evidence of relapse compared to a subject to which the anti-ADAM17 biologic is not administered, decreasing the severity of symptoms and/or clinical signs of relapse, and/or completely resolving the condition. Treatment initiated while the subject exhibits a symptom or clinical sign associated with the condition may result in decreasing the severity of symptoms and/or clinical signs of the condition compared to a subject to which the anti-ADAM17 biologic is not administered, and/or completely resolving the condition.

The amount of an anti-ADAM17 biologic administered can vary depending on various factors including, but not limited to, the specific anti-ADAM17 biologic being administered, the weight, physical condition, and/or age of the subject, and/or the route of administration. Thus, the absolute weight of anti-ADAM17 biologic included in a given unit dosage form can vary widely, and depends upon factors such as the species, age, weight, and physical condition of the subject, and/or the method of administration. Accordingly, it is not practical to set forth generally the amount that constitutes an amount of anti-ADAM17 biologic effective for all possible applications. Those of ordinary skill in the art, however, can readily determine the appropriate amount with due consideration of such factors.

In one or more embodiments, the method can include administering sufficient anti-ADAM17 biologic to provide a dose of, for example, from about 100 ng/kg to about 50 mg/kg to the subject, although In one or more embodiments the methods may be performed by administering anti-ADAM17 biologic in a dose outside this range.

In one or more embodiments, the method can includes administering sufficient anti-ADAM17 biologic to provide a minimum dose of at least 100 ng/kg such as, for example, at least 1 μg/kg, at least 5 μg/kg, at least 10 μg/kg, at least 25 μg/kg, at least 50 μg/kg, at least 100 μg/kg, at least 200 μg/kg, at least 300 μg/kg, at least 400 μg/kg, at least 500 μg/kg, at least 600 μg/kg, at least 700 μg/kg, at least 800 μg/kg, at least 900 μg/kg, or at least 1 mg/kg.

In one or more embodiments, the method includes administering sufficient anti-ADAM17 biologic to provide a maximum dose of no more than 10 mg/kg such as, for example, no more than 5 mg/kg, no more than 4 mg/kg, no more than 3 mg/kg, no more than 2 mg/kg, no more than 1 mg/kg, no more than 900 μg/kg, no more than 800 μg/kg, no more than 700 μg/kg, no more than 600 μg/kg, no more than 500 μg/kg, no more than 400 μg/kg, no more than 300 μg/kg, no more than 200 μg/kg, no more than 100 μg/kg, no more than 90 μg/kg, no more than 80 μg/kg, no more than 70 μg/kg, no more than 60 μg/kg, no more than 50 μg/kg, no more than 40 μg/kg, no more than 30 μg/kg, no more than 20 μg/kg, or no more than 10 μg/kg/day. The anti-ADAM17 biologic provides a dose of “no greater than” a specified amount when the anti-ADAM17 biologic is not absent but is present in an amount up to and including the specified amount.

In one or more embodiments, the method includes administering sufficient anti-ADAM17 biologic to provide a dose characterized by a range having endpoints defined by any a minimum dose identified above and any maximum dose that is greater than the selected minimum dose. For example, In one or more embodiments, the method can include administering sufficient anti-ADAM17 biologic to provide a dose of from about 10 μg/kg to about 10 mg/kg to the subject, a dose of from about 100 μg/kg to about 1 mg/kg, a dose of from g/kg to 100 μg/kg, etc.

In certain embodiments, the method includes administering sufficient anti-ADAM17 biologic to provide a dose that is equal to any minimum dose or any maximum dose listed above. Thus, for example, in certain embodiments, the method can include administering sufficient anti-ADAM17 biologic to provide a dose of 1 μg/kg, 5 μg/kg, 10 μg/kg, 25 μg/kg, 50 μg/kg, 100 μg/kg, 200 μg/kg, 500 μg/kg, 1 mg/kg, 5 mg/kg, etc.

In one or more embodiments, an anti-ADAM17 biologic may be administered, for example, from a single dose to multiple doses per week, although In one or more embodiments the method can be performed by administering an anti-ADAM17 biologic at a frequency outside this range. In certain embodiments, an anti-ADAM17 biologic may be administered from about once per month to about five times per week. In one or more embodiments, a dose may be described in terms of the amount of anti-ADAM17 biologic administered over a 24-hour period, which may be administered in a seven-day cycle of, for example, four days of treatment and three days of rest.

In one or more embodiments, an anti-ADAM17 biologic may be administered, for example, from a single dose to multiple cycles of treatment, although In one or more embodiments the method can be performed by administering an anti-ADAM17 biologic for a duration outside this range. In one or more embodiments, the anti-ADAM17 biologic may be administered for three weeks. In such embodiments, each week may be a treatment cycle such as the exemplary treatment cycle described in the preceding paragraph. In other embodiments, the anti-ADAM17 biologic may be administered for a greater number of treatment cycles, without a gap between one set of treatment cycles and a subsequent set of treatment cycles. The gap between one set of treatment cycles and a subsequent set of treatment cycles may be a gap of one or more weeks, one or more months, or one or more years.

In one or more embodiments, the method further includes administering one or more additional therapeutic agents. The one or more additional therapeutic agents (e.g., chemotherapeutic agents) may be administered before, after, and/or coincident to the administration of an anti-ADAM17 biologic. An anti-ADAM17 biologic and the additional therapeutic agents may be co-administered. As used herein, “co-administered” refers to two or more components of a combination administered so that the therapeutic or prophylactic effects of the combination can be greater than the therapeutic or prophylactic effects of either component administered alone. Two components may be co-administered simultaneously or sequentially. Simultaneously co-administered components may be provided in one or more pharmaceutical compositions. Sequential co-administration of two or more components includes cases in which the components are administered so that each component can be present at the treatment site at the same time. Alternatively, sequential co-administration of two components can include cases in which at least one component has been cleared from a treatment site, but at least one cellular effect of administering the component (e.g., cytokine production, activation of a certain cell population, etc.) persists at the treatment site until one or more additional components are administered to the treatment site. Thus, a co-administered combination can, in certain circumstances, include components that never exist in a chemical mixture with one another. In other embodiments, the anti-ADAM17 biologic and the additional therapeutic agent may be administered as part of a mixture or cocktail. In some aspects, the administration of anti-ADAM17 biologic may allow for the effectiveness of a lower dosage of other therapeutic modalities when compared to the administration of the other therapeutic agent or agents alone, thereby decreasing the likelihood, severity, and/or extent of the toxicity observed when a higher dose of the other therapeutic agent or agents is administered.

Exemplary additional therapeutic agents include altretamine, amsacrine, L-asparaginase, colaspase, bleomycin, busulfan, capecitabine, carboplatin, carmustine, chlorambucil, cisplatin, cladribine, cyclophosphamide, cytophosphane, cytarabine, dacarbazine, dactinomycin, daunorubicin, docetaxel, doxorubicin, epirubicin, etoposide, fluorouracil, fludarabine, 10 fotemustine, ganciclovir, gemcitabine, hydroxyurea, idarubicin, ifosfamide, irinotecan, lomustine, melphalan, mercaptopurine, methotrexate, mitoxantrone, mitomycin C, nimustine, oxaliplatin, paclitaxel, pemetrexed, procarbazine, raltitrexed, temozolomide, teniposide, tioguanine, thiotepa, topotecan, vinblastine, vincristine, vindesine, and vinorelbine.

In one or more embodiments, administering the anti-ADAM17 biologic and at least one additional therapeutic agent produces therapeutic synergy. In some aspects of the methods of the present invention, a measurement of the response to treatment observed after administering both an anti-ADAM17 biologic as described herein and the additional therapeutic agent is improved over the same measurement of response to treatment observed after administering either the anti-ADAM17 biologic or the additional therapeutic agent alone.

### Treatment of Infection by SARS-CoV-2

In yet another aspect, an anti-ADAM17 antibody as described herein, either alone, incorporated into an anti-ADAM17 biological, incorporated into a TAB compound, or in combination with one or more additional active agents, can be used therapeutically to treat SARS-CoV-2 infection. The anti-ADAM17 antibody can include the amino acid sequence of one or more of SEQ ID NO:12, SEQ ID NO:16, SEQ ID NO:20, SEQ ID NO:21, SEQ ID NO:22, or SEQ ID NO:23. Alternatively, the anti-ADAM17 antibody can include an antibody fragment that includes one or more of the CDRs reflected in SEQ ID NO:13, SEQ ID NO:14, SEQ ID NO:15, SEQ ID NO:17, SEQ ID NO:18, or SEQ ID NO:19.

The SARS-CoV-2 virus infects host cells by binding to host Angiotensin Converting Enzyme 2 (ACE2) expressed at epithelial surfaces. ACE2 is a dimeric, zinc-dependent metalloprotease that cleaves several substrates. ACE2 is a substrate of ADAM17. Thus, ADAM17 is involved in regulating ACE2 expression and, potentially, SARS-CoV-2 infection. Because ADAM17 alters the expression of surface ACE2 and the generation of soluble ACE2 (sACE2), its activity likely affects SARS-CoV-2 virus infection. The loss of ACE2 from epithelial cell surfaces due to ADAM17 sheddase activity may also further dampen viral spread. This disclosure describes the effect of ADAM17 on both disease outcome and viral burden in K18-hACE2 mice infected with SARS-CoV-2. Mice treated with an anti-ADAM17 monoclonal antibody displayed less morbidity than saline-treated mice, appeared healthier, and lost significantly less weight than the saline-treated group. Antibody-treated mice displayed significantly less overall damage and infiltrate in the lung compared to saline-treated mice.

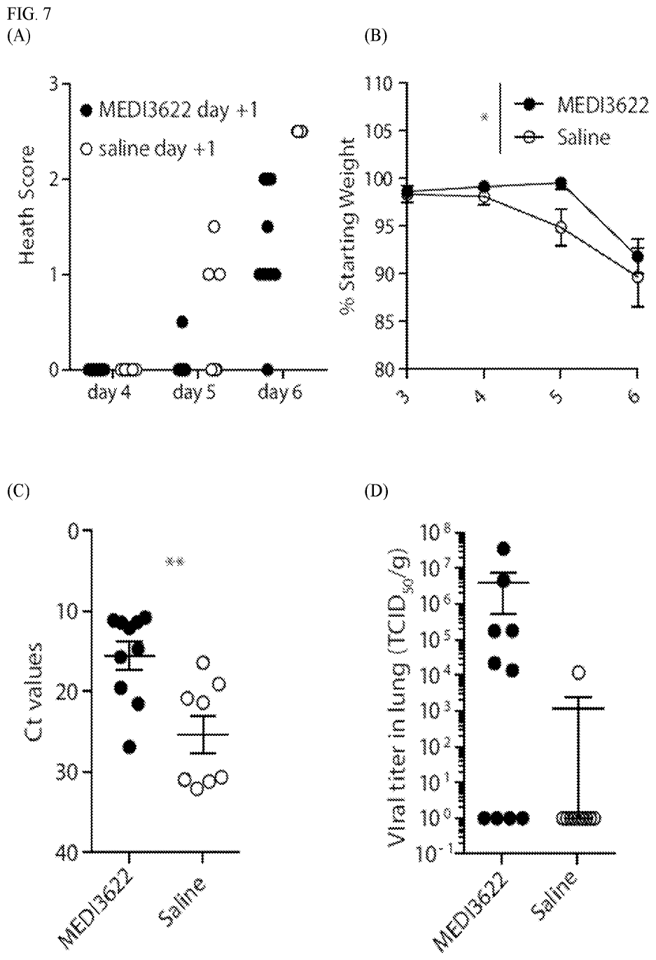

The highly specific anti-human and anti-mouse ADAM17 function-blocking mAb MEDI3622 (Rios-Doria et al., Mol Cancer Ther. 2015; 14(7):1637-1649; Mishra et al., Cancer Immunol Immunother. 2018; 67(9):1407-1416) was used to test the effects of an anti-ADAM17 antibody on disease progression and virus replication in SARS-CoV-2 infected human ACE2 transgenic mice (K18-hACE2). During a 6-day infection, saline-treated mice displayed greater morbidity based on a health scoring system (FIG. 7A) and two out of 10 of these mice succumbed to infection prior to the end of the 6-day experiment. In contrast, anti-ADAM17 antibody-treated mice appeared healthier, and all mice survived. Furthermore, saline-treated mice lost significantly more weight than mice in the anti-ADAM17 antibody-treated group (FIG. 7B). These data indicate that inhibition of ADAM17 activity during SARS-CoV-2 infection reduced morbidity.

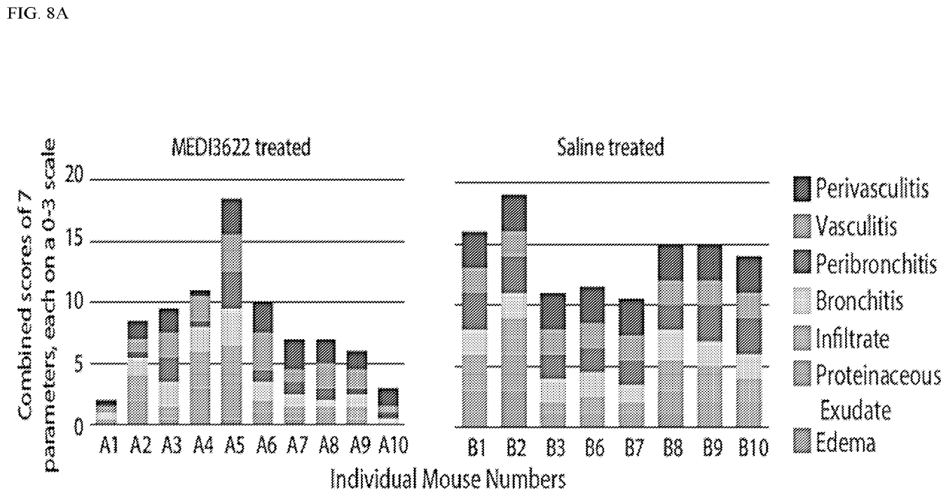

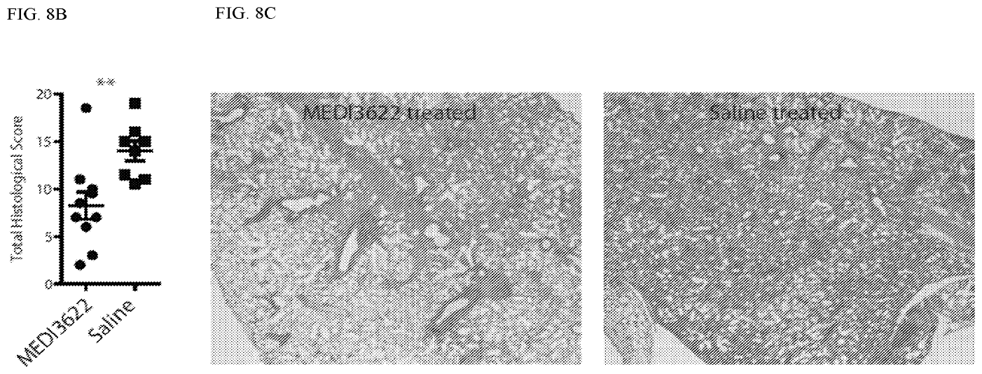

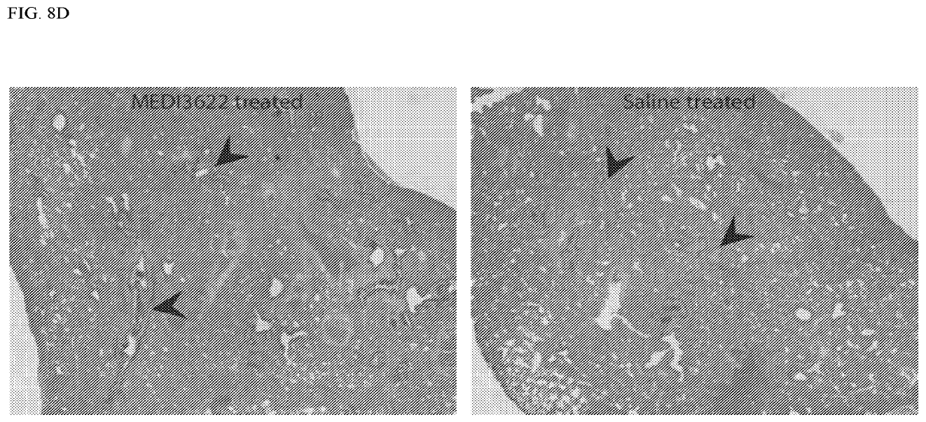

Histological analyses were performed to assess damage and inflammation in lung tissue. Tissue sections were hematoxylin and eosin (H & E)-stained and scored based on seven unique inflammatory parameters allowing quantification of overall damage. As shown in FIG. 8A and FIG. 8B the lungs from anti-ADAM17 antibody-treated mice displayed significantly less overall damage and inflammatory cell infiltrate than those from saline-treated mice. Furthermore, representative sections from each group in FIG. 8C qualitatively support the histological scores noted above. Finally, collapsed large bronchi were observed in the lungs of seven out of eight saline-treated mice, and only three of 10 anti-ADAM17 antibody-treated mice (FIG. 8D). Thus, the lung histopathology is consistent with greater morbidity and mortality of saline-treated mice compared to those treated with an anti-ADAM17 antibody.

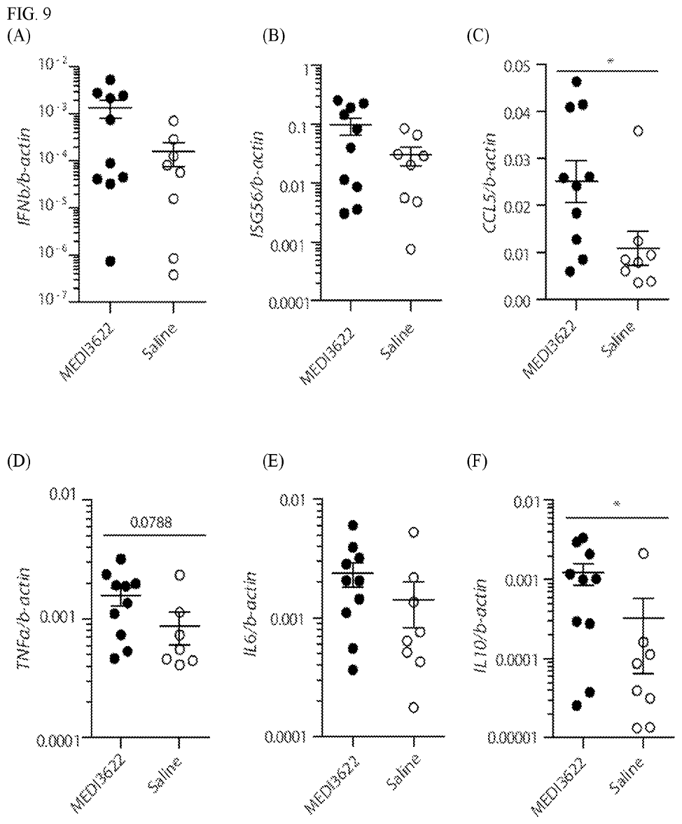

To quantify changes in pro-inflammatory and anti-inflammatory gene transcription, qPCR was used to measure relative expression of a number of cytokine and chemokine transcripts in the lung. Despite differences in cellular inflammation, there were either no differences between the groups or a slight increase in the anti-ADAM17 antibody-treated mice for most of the transcripts. FIG. 9 shows that the antiviral transcripts Ifnb and downstream Isg56, as well as transcripts encoding the chemokine Ccl5 and inflammatory cytokines Tnfa and Il6 were slightly increased in lungs of anti-ADAM17 antibody-treated mice, compared to saline-treated mice. Il10 transcript expression was also increased, which may suggest an anti-inflammatory control mechanism. These data may suggest that antibody treatment inhibited and/or delayed the early expression of transcripts indicative of cytokine storm, whereas in saline-treated mice this response perhaps occurred earlier in infection resulting in extensive lung damage. Increased expression of transcripts encoding Ifnb and Isg56 suggest that anti-viral defenses were not compromised in the anti-ADAM17 antibody-treated animals.

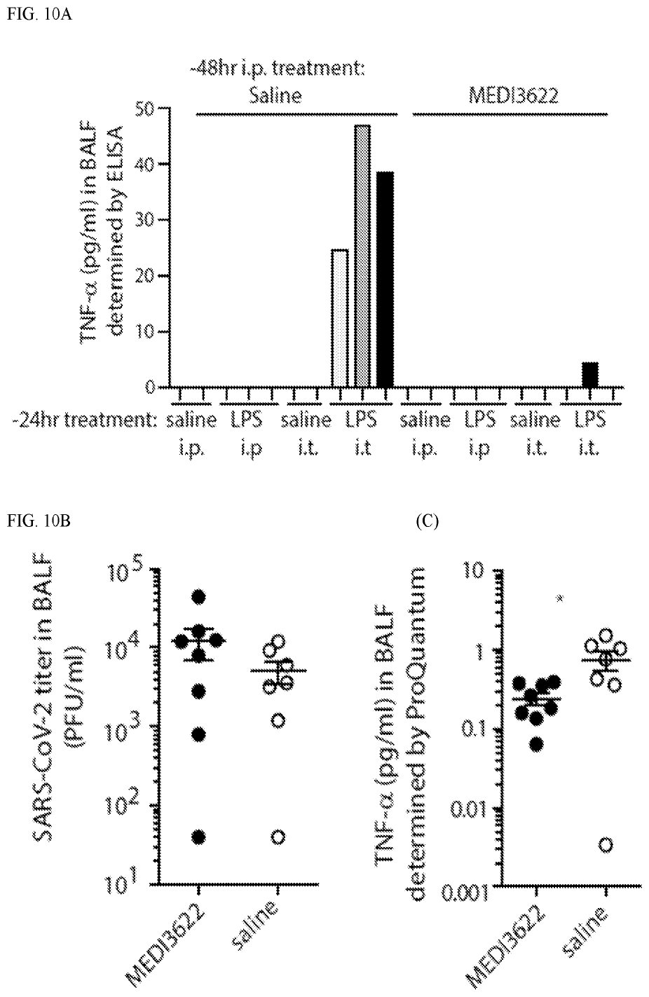

Finally, anti-ADAM17 antibody treatment of K18 hACE2-expressing mice decreases soluble TNF-α in the lung. ADAM17 is a well-established sheddase of TNF-α and to establish that this occurred in K18 hACE2 mice, they were treated with either anti-ADAM17 antibody or saline by intraperitoneal injection and E. coli LPS (1 μg/mouse) or saline delivered either intraperitoneally or intratracheally to stimulate rapid production of inflammatory cytokines, including TNF-α. Twenty-four hours later, the mice were euthanized and BALF and blood were collected. Murine TNF-α was quantified in BALF and in plasma by ELISA. LPS treatment in the lung produced soluble TNF-α detectible in the BALF, but prior treatment with anti-ADAM17 antibody (delivered intraperitoneally) effectively blocked this production (FIG. 10A). This appears to be a lung-specific effect, as TNF-α was not detectible in the serum of any mice, regardless of route of LPS delivery.

A similar experiment was performed with SARS-CoV-2 infected mice. These mice were infected then treated with either anti-ADAM17 antibody or saline and euthanized four days after infection, well before onset of symptomatic disease. BALF was collected and assessed for infectious virus and TNF-α. At this interval, a slight increase in SARS-CoV-2 was already detectible by plaque assay in BALF from anti-ADAM17 antibody-treated mice, but was not significant (FIG. 10B). Soluble TNF-α in BALF was measured by a highly sensitive qPCR-based protein detection assay. FIG. 10C shows that treatment with anti-ADAM17 antibody resulted in a significant reduction in TNF-α detection. This is in contrast to Tnfa transcripts, which were increased in the lungs of MEDI3622-treated mice (FIG. 9). Hence, our findings demonstrate that for ADAM17 substrates, an upregulation in their gene expression does not necessarily indicate increased release of their soluble form. Blocking of TNF-α shedding in the lungs of SARS-CoV-2 infected mice by treatment with anti-ADAM17 antibody likely diminishes inflammation and morbidity in the mouse model of COVID-19.

In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

NK92 cells (a human NK cell line) expressing wildtype CD16A, a well-described ADAM17 substrate, were stimulated (2.5×10⁶/ml in PBS) with 15 ng/mL phorbol-12-myristate-13-acetate (PMA, MilliporeSigma, Burlington, MA) for 30 minutes at 37° C., which was stopped by cell washing with PBS (Lonza, Basel, Switzerland) at 4° C. Some cells were pre-incubated for 30 minutes on ice with an anti-ADAM17 scFv, a negative control scFv (anti-fibroblast-associated protein (FAP)), a hydroximate-based small molecule inhibitor of ADAM17 (TAPI-1, MilliporeSigma, Burlington, MA), or the ADAM17 function blocking mAb D1(A12) (Sigma-Aldrich). For cell staining, nonspecific antibody binding sites were first blocked and then cells were stained with the anti-human CD16 mAb 3G8 (BioLegend, San Diego, CA). A Zombie viability kit (Invitrogen, Carlsbad, CA) was used to assess live versus dead cells. An FSC-A/SSC-A plot was used to set an electronic gate on viable NK92 cells (experiment 1, panel 1). An FSC-A/FSC-H and SSC-A/SSC-H plot was used to set an electronic gate on single cells. All cell staining was analyzed on a FACSCelesta instrument (BD Biosciences, San Jose, CA).

### Example 2

### Reagents

The anti-ADAM17 mAb MEDI3622 (human IgG1) has been previously described (Rios-Doria et al., Mol Cancer Ther. 2015; 14(7):1637-1649; Mishra et al., Cancer Immunol Immunother. 2018; 67(9):1407-1416). All commercially available mAbs are listed in Table 1. Recombinant human (rh) IL-15 was obtained from the Biological Resources Branch, NCI, NIH and from R&D Systems, Inc. (Minneapolis, MN).

TABLE 1
Commercial antibodies used

| | | Catalogue | |
|---|---|---|---|
| Antigen | Clone | # | Company |
| CD56 | HCD56 | 318318 | BioLegend, San |
| | | | Diego, CA |
| CD3 | HIT3a | 300440 | BioLegend |
| CD16 | 3G8 | 302038 | BioLegend |
| CD336/NKp44 | P44-8 | 325108 | BioLegend |
| CD335/NKp46 | 9E2 | 331914 | BioLegend |
| CD159a/NKG2A | Z199 | A60797 | Beckman Coulter, |
| | | | Brea, CA |
| CD314/NKG2D | 1D11 | 320806 | BioLegend |
| CD158a/KIR2DL1 | HP-MA4 | 339504 | BioLegend |
| CD158b1/KIR2DL2/L3 | DX27 | 312612 | BioLegend |
| CD158e1/KIR3DL1 | DX9 | 312714 | BioLegend |
| CD45 | HI30 | 304044 | BioLegend |
| CD62L/L-selectin | DREG56 | 304810 | Biolegend |
| CD62L/L-selectin | DREG200 | HB302 | ATCC, Manassas, VA |
| CD156b/ADAM17 | D1(A12) | AB00611- | Absolute Antibody |
| | | 10.0 | Limited, Oxford, UK |
| In vivo grade isotype | CB1 | C0001 | Crown Bioscience, |
| control, human IgG1 | | | San Diego, CA |
| In vivo grade isotype | CB5 | C0005 | Crown Bioscience |
| control, mouse IgG1 | | | |

### NK Cell Isolation

Fresh human peripheral blood leukocytes from platelet pheresis were obtained from Innovative Blood Resources (St. Paul, MN). PBMCs were further enriched by Ficoll-Paque Plus (GE Healthcare Bio-Sciences AB, Uppsala, Sweden) gradient and then NK cells were purified by negative depletion using isolation kits from StemCell Technologies (Cambridge, MA) or Miltenyi Biotec (Auburn, CA), according to the manufacturer's instructions, with >95% viability and >85% enrichment of CD56⁺CD3⁻ lymphocytes. Viable cell counting was performed using a Countess II automated cell counter (Life Technologies Corporation, Bothell, WA).

### In Vitro NK Cell Proliferation

Enriched NK cells were labeled with CellTrace Violet Cell Proliferation Dye (ThermoFisher Scientific, Inc., Waltham, MA) per manufacturer's instructions and incubated for seven days in media containing or lacking rhIL-15 (R&D Systems, Inc., Minneapolis, MN) at the indicated concentration in the presence or absence of the indicated mAbs. NK cells (CD56⁺CD3⁻) were analyzed for dilution of dye by flow cytometry. Expansion index was calculated using FlowJo software (FlowJo, Ashland, OR) and is the predicted fold expansion of the overall culture based on CellTrace Violet dilution.

### Xenogeneic NK Cell Adoptive Transfer

These procedures were performed as previously described (Miller et al., Biol Blood Marrow Transplant. 2014; 20(8):1252-1257). All animal procedures were approved by the Institutional Animal Care and Use Committee at the University of Minnesota. NOD-scid IL2Rgamma^{null} (NSG) mice (stock number is 005557 from Jackson Laboratory, Bar Harbor, ME) were housed in a specific pathogen-free facility. Weight matched (26-30 g) mice underwent whole-body preconditioning irradiation (225 cGy using an X RAD 320, Precision X-ray, North Branford, CT, USA) for consistent NK cell engraftment. Freshly, enriched human NK cells underwent initial overnight incubation in BO media [DMEM, Ham's F12 with 10% human AB serum, Pen/Strep (1%), 2-ME (20 μm), ethanolamine (50 μm), ascorbic Acid (10 μg/ml) and sodium selenite (1.6 ng/ml)] along with 2.5 ng/ml rhIL-15 (NCI). NK cells (4×10⁶) were injected via tail vein in each mouse. Mice were administered rhIL-15 (NCI) ip at dose of 5 μg. The indicated mAbs were administered intraperitoneally at a dose of 10 mg/kg. Blood was collected via retro-orbital route in heparin. Spleen samples were subjected to single-cell suspension using GentleMACS C tubes on a gentleMACS™ Dissociator (Miltenyi Biotech, Bergisch Gladbach, Germany), per the manufacturer's instructions. Absolute counting of human NK cells in the peripheral blood and spleen was performed on a flow cytometer using a bead counting method (AccuCheck, Thermo Scientific, Waltham, MA, USA) according to the manufacturer's instructions.

### Flow Cytometric Analyses

NK cells were stained with the indicated antibodies and examined by flow cytometry, as previously described (Mishra et al., Cancer Immunol Immunother. 2018; 67(9):1407-1416). For controls, fluorescence minus one was used as well as appropriate isotype-matched antibodies since NK cells express Fc receptors. An FSC-A/SSC-A plot was used to set an electronic gate on leukocyte populations, and an FSC-A/FSC-H plot was used to set an electronic gate on single cells. 7AAD was used to assess live vs. dead cells, as per the manufacturer's instructions (BioLegend, San Diego, CA).

### Statistical Analysis

All comparisons between two groups were deemed statistically significant by unpaired two-tailed Student's t-test if p<0.05. (*) denotes p<0.05, (**) denotes p<0.001.

### Example 3

### Mice and Infection

All animal studies were reviewed and approved by the Montana State University IACUC committee. All experiments with SARS-CoV-2 were performed under BSL3 containment. All mice were purchased from Jackson Laboratory, Bar Harbor, ME. K18-hACE2 transgenic mice ([B6.Cg-Tg(K18-ACE2)2Prlmn/J, Stock No: 034860] express human ACE2 (hACE2), the receptor used by SARS coronaviruses to gain cellular entry. The K18 promoter directs expression to epithelia, including airway epithelia where infections typically begin. SARS-CoV-2 (WA/01 strain, BEI Resources) was propagated on Vero E6 cells for three passages as previously described (Loveday et al., Viruses 13:562, 2021) and quantified by endpoint titration and plaque assay. This strain was sequenced and found to have a deletion in the furin cleavage site known to promote viral infection. At infection, mice were eight-to-eleven weeks old. Mice were lightly anesthetized with isoflurane and oxygen, and given 10⁴ PFU SARS-CoV-2 in 30 μl by the intranasal route. This infectious dose induced disease that mimics moderate to severe COVID-19 in humans. On day one post-infection, mice were treated intraperitoneally with either 15 mg/kg MEDI3622 mAb (MEDI3622; Rose-Doria et al., Mol Cancer Ther 14(7):1637-1649, 2015) or an equal volume of saline vehicle. Dosing of MEDI3622 was based on similar studies involving a mouse sepsis model (Mishra et al., Int. J. Mol Sciences 7:138, 2020). Mice were weighed and their health was scored daily on a scale of 0-3 (3=moribund, 20% weight loss; 2=15% weight loss, AND hunched posture, possible build-up around eyes, impaired mobility, matted fur; 1=10% weight loss, slight (if any) impaired mobility; 0=no symptoms, no weight loss). Mice were euthanized by pentobarbital injection followed by exsanguination on either day 4 or day 6 post-infection. Bronchoalveolar lavage fluids (BALF) and blood were collected from mice euthanized at day 4 post infection. At day 6 post-infection, blood and whole lungs were collected for histology, RNA extraction and virus quantification.

Viral Quantification (qPCR/Plaque Assay)

For mice euthanized at day 6 post infection, all portions of the lung besides the left lobe were homogenized in 2 ml of HBSS. Following homogenization, 1 ml was centrifuged at 500×g for five minutes, and the cell pellet frozen at −80° C. After thawing, the pellet was resuspended in 350 μl buffer RLT (QIAmp Viral RNA Mini Kit 52906) and RLT-lysed lung homogenate was heat inactivated for at least 20 minutes at 65° C. to allow safe removal from BSL3 containment. Homogenized lung lysate was processed through a QIAshredder column and RNA was extracted using the QIAGEN RNeasy Mini kit. RT-qPCR was performed with SARS-CoV-2 (2019-nCoV) CDC RUO Primers and Probes purchased from IDT and Quantabio UltraPlex 1-Step ToughMix RT-qPCR mix.

Virus in lung homogenate supernatant fluids and BALF was measured by plaque assay as previously described (Loveday et al., Viruses 13:562, 2021). Briefly, fluids were 10-fold serially diluted out to 10⁻⁵ and 300 μl was applied to confluent monolayers of low passage Vero E6 cells in a six-well flat-bottomed plate. The plates were incubated for one hour with occasional gentle rocking, then overlayed with 1% methylcellulose in complete DMEM medium. The plates were incubated for four days, then the supernatant fluids were removed, and the cell monolayers were stained with 0.5% methylene blue in 70% ethanol to reveal plaques. Plaques were counted and PFU/ml calculated.

### Transcript Analysis

Lung RNA was extracted as described above. RNA was reverse-transcribed using the iScript cDNA Synthesis kit (Bio-Rad Laboratories, Inc., Hercules, CA) and gene expression was measured using AzuraQuant Green Fast qPCR Mix (Azura Genomics AZ-2105) and primers targeting genes of interest. Primer sequences are as follows:

ifnb
(forward, SEQ ID NO: 53)
5′-AAGAGTTACACTGCCTTTGCCATC-3′
and
(reverse, SEQ ID NO: 54)
5′-CACTGTCTGCTGGTGGAGTTCATC-3′;
ccl5
(forward, SEQ ID NO: 55)
5′-GGGTACCATGAAGATCTCTGCA-3′
and
(reverse, SEQ ID NO: 56)
5′-TTGGCGGTTCCTTCGAGTGA-3′;
il6
(forward, SEQ ID NO: 57)
5′-TAGTCCTTCCTACCCCAATTTCC-3′
and
(reverse, SEQ ID NO: 58)
5′-TTGGTCCTTAGCCACTCCTTC-3′;
tnfα
(forward, SEQ ID NO: 59)
5′-AAGCCTGTAGCCCACGTCGTA-3′
and
(reverse, SEQ ID NO: 60)
5′-GGCACCACTAGTTGGTTGTCTTTG-3′;
il10
(forward, SEQ ID NO: 61)
5′-GCTCTTACTGACTGGCATGAG-3′
and
(reverse, SEQ ID NO: 62)
5′-CGCAGCTCTAGGAGCATGTG-3.

### TNF-α Detection

TNF-α was measured in BALF from mice treated with MEDI3622 or saline following either LPS treatment or four days after SARS-CoV-2 infection. Mouse TNF-α in LPS-treated mice was measured using ELISA MAX Deluxe Set Mouse TNF-α (Biolegend, Inc., San Diego, CA). TNF-α in BALF collected day 4 post-infection from SARS-CoV-2 infected mice was measured using the PROQUANTUM Mouse TNF-α Immunoassay Kit (Invitrogen, Carlsbad, CA).

### Histology

The left lobe of the lung was collected at sacrifice and fixed in 10% formalin, paraffin embedded, sectioned into 5 μm sections, and stained with hematoxylin and eosin (H & E) following the manufacturer's instructions. Histological sections were scored based on a 0-3 scale (0: none; 1: mild; 2: moderate; 3: severe) for seven separate parameters: infiltration across the parenchyma, bronchitis, peribronchitis, vasculitis, perivasculitis, proteinaceous exudate, and edema. Images were acquired using a DS-Ri-1 camera (Nikon, Tokyo, Japan) mounted on a Nikon Eclipse 80i microscope at 4× magnification and analyzed by a blinded histotechnician.

### Statistical Analyses

Statistical analyses were performed using Prism (GraphPad Software, San Diego, CA). The Kolmogorov-Smirnov test (with Dallal-Wilkinson-Liliefor p-value) was used to determine if the data formed a Gaussian distribution. The data were found to be normally distributed, thus the two groups were compared using the unpaired students t test. Data were considered significant when p<0.05.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

Sequence Listing Free Text

Targeting
SEQ ID NO: 1 - COVID-19 receptor binding domain (RBD)
TNLCPFGEVF NATRFASVYA WNRKRISNCV ADYSVLYNSA SFSTFKCYGV SPTKLNDLCF
TNVYADSFVI RGDEVRQIAP GQTGKIADYN YKLPDDFTGC VIAWNSNNLD SKVGGNYNYL
YRLFRKSNLK PFERDISTEI YQAGSTPCNG VEGFNCYFPL QSYGFQPTNG VGYQPYRVVV
LSFELLHAPA TVCGPKK
SEQ ID NO: 2 - sdAb against CD16 (cam16):
QVQLVESGGG LVQPGGSLRL SCAASGLTFS SYNMGWFRQA PGQGLEAVAS ITWSGRDTFY
ADSVKGRFTI SRDNSKNTLY LQMNSLRAED TAVYYCAANP WPVAAPRSGT YWGQGTLVTV
SS
SEQ ID NO: 3 - scFv against CD19:
DILLTQTPAS LAVSLGQRAT ISCKASQSVD YDGDSYLNWY QQIPGQPPKL LIYDASNLVS
GIPPRFSGSG SGTDFTLNIH PVEKVDAATY HCQQSTEDPW TFGGGTKLEI KRGSTSGSGK
PGSGEGSTKG QVQLQQSGAE LVRPGSSVKI SCKASGYAFS SYWMNWVKQR PGQGLEWIGQ
IWPGDGDTNY NGKFKGKATL TADESSSTAY MQLSSLASED SAVYFCARRE TTTVGRYYYA
MDYWGQGTSV TVSS
SEQ ID NO: 4 - anti-vegf (V_{H}_Whitlow_Linker_V_{L}):
EVQLVESGGG LVQPGGSLRL SCAASGYTFT NYGMNWVRQA PGKGLEWVGW INTYTGEPTY
AADFKRRFTF SLDTSKSTAY LQMNSLRAED TAVYYCAKYP HYYGSSHWYF DVWGQGTLVT
VSGSTSGSGK PGSGEGSTKG DIQMTQSPSS LSASVGDRVT ITCSASQDIS NYLNWYQQKP
GKAPKVLIYF TSSLHSGVPS RFSGSGSGTD FTLTISSLQP EDFATYYCQQ YSTVPWTFGQ
GTKVEIK
SEQ ID NO: 5 - anti-Her2 (V_{H}_Whitlow_Linker_V_{L}):
EVQLVESGGG LVQPGGSLRL SCAASGFNIK DTYIHWVRQA PGKGLEWVAR IYPTNGYTRY
ADSVKGRFTI SADTSKNTAY LQMNSLRAED TAVYYCSRWG GDGFYAMDYW GQGTLVTVSS
GSTSGSGKPG SGEGSTKGDI QMTQSPSSLS ASVGDRVTIT CRASQDVNTA VAWYQQKPGK
APKLLIYSAS FLYSGVPSRF SGSRSGTDFT LTISSLQPED FATYYCQQHY TTPPTFGQGT
KVEIKRTV
SEQ ID NO: 6 - anti-EGFR (V_{H}_Whitlow_Linker_V_{L}):
QVQLKQSGPG LVQPSQSLSI TCTVSGFSLT NYGVHWVRQS PGKGLEWLGV IWSGGNTDYN
TPFTSRLSIN KDNSKSQVFF KMNSLQSNDT AIYYCARALT YYDYEFAYWG QGTLVTVSAG
STSGSGKPGS GEGSTKGDIL LTQSPVILSV SPGERVSFSC RASQSIGTNI HWYQQRTNGS
PRLLIKYASE SISGIPSRFS GSGSGTDFTL SINSVESEDI ADYYCQQNNN WPTTFGAGTK
LELKRTV
SEQ ID NO: 7 - anti-EpCAM
DIQMTQSPSS LSASVGDRVT ITCRSTKSLL HSNGITYLYW YQQKPGKAPK LLIYQMSNLA
SGVPSRFSSS GSGTDFTLTI SSLQPEDFAT YYCAQNLEIP RTFGQGTKVE LKRATPSHNS
HQVPSAGGPT ANSGTSGEVQ LVQSGPGLVQ PGGSVRISCA ASGYTFTNYG MNWVKQAPGK
GLEWMGWINT YTGESTYADS FKGRFTFSLD TSASAAYLQI NSLRAEDTAV YYCARFAIKG
DYWGQGTLLT VSS
SEQ ID NO: 8 - anti-B7H3 (clone 1)
HVQLVESGGG LVQPGRSLRL SCAASGFTFS SYWMYWVRQT PGKGLEWVST INRDGSATWY
ADSVKGRFTI SRDNAKNTGY LQMNSLEPDD TAVYYCVSDP DNYSSDEMVP YWGQGTQVTV
SS
SEQ ID NO: 9 - antiB7H3 (clone 2)
QVQLVESGGG LVQPGGSLRL SCAASGFTFS SYWMYWVRQT PGKGLEWVST INRDGSATWY
ADSVKGRFTI SRDNAKNTGY LQMNSLKPDD TAVYYCVSDP DNYSSDEMVP YWGQGTQVTV
SS
SEQ ID NO: 10 - anti-CD3 (OKT3)
QVQLQQSGAE LARPGASVKM SCKASGYTFT RYTMHWVKQR PGQGLEWIGY INPSRGYTNY
NQKFKDKATL TTDKSSSTAY MQLSSLTSED SAVYYCARYY DDHYCLDYWG QGTTLTVSSG
GGGSGGGGSG GGGSQIVLTQ SPAIMSASPG EKVTMTCSAS SSVSYMNWYQ QKSGTSPKRW
IYDTSKLASG VPAHFRGSGS GTSYSLTISG MEAEDAATYY CQQWSSNPFT FGSGTKLEIN
R
SEQ ID NO: 11 - anti-CD3 (UCHT)
EVQLQQSGPE LVKPGASMKI SCKASGYSFT GYTMNWVKQS HGKNLEWMGL INPYKGVSTY
NQKFKDKATL TVDKSSSTAY MELLSLTSED SAVYYCARSG YYGDSDWYFD VWGQGTTLTV
FSGGGGSGGG GSGGGGSDIQ MTQTTSSLSA SLGDRVTISC RASQDIRNYL NWYQQKPDGT
VKLLIYYTSR LHSGVPSKFS GSGSGTDYSL TISNLEQEDI ATYFCQQGNT LPWTFAGGTK
LEIK
ADAM17-inhibiting
SEQ ID NO: 12 - V_{L}
DIQMTQSPSS LSASVGDRVT ITCRSSQSIP SYLNWYQQKP GKAPKLLIYA ASRLQSGVPS
RFSGSGSGTD FTLTISSLQP EDFATYYCQQ SYSTPLTFGG
GTKVEIK
CDR1: amino acids 24-34
CDR2: amino acids 50-56
CDR3: amino acids 89-97
SEQ ID NO: 13 - V_{L} CDR1
RSSQSIPSYL N
SEQ ID NO: 14 - V_{L} CDR2
AASRLQS
SEQ ID NO: 15 - V_{L} CDR3
QQSYSTPLT
SEQ ID NO: 16 - V_{H}
EVQLLESGGG LVQPGGSLRL SCAASGFTFS SYPMNWVRQA PGKGLEWVSY ISPFGGMTDY
ATSVKGRFTI SRDNSKNTLY LQMNSLRAED TAVYYCARDA MRGAEVDYWG QGTLVTVSS
CDR1: amino acids 26-32
CDR2: amino acids 52-57
CDR3: amino acids 99-108
SEQ ID NO: 17 - V_{H} CDR1
GFTFSSY
SEQ ID NO: 18 - V_{H} CDR2
SPFGGM
SEQ ID NO: 19- V_{H} CDR3
DAMRGAEVDY
SEQ ID NO: 20 - Human IgG4 V_{H}
MDPKGSLSWR ILLFLSLAFE LSYGEVQLLE SGGGLVQPGG SLRLSCAASG FTFSSYPMNW
VRQAPGKGLE WVSYISPFGG MTDYATSVKG RFTISRDNSK NTLYLQMNSL RAEDTAVYYC
ARDAMRGAEV DYWGQGTLVT VSSASTKGPS VFPLAPCSRS TSESTAALGC LVKDYFPEPV
TVSWNSGALT SGVHTFPAVL QSSGLYSLSS VVTVPSSSLG TKTYTCNVDH KPSNTKVDKR
VESKYGPPCP PCPAPEFLGG PSVFLFPPKP KDTLMISRTP EVTCVVVDVS QEDPEVQFNW
YVDGVEVHNA KTKPREEQFN STYRVVSVLT VLHQDWLNGK EYKCKVSNKG LPSSIEKTIS
KAKGQPREPQ VYTLPPSQEE MTKNQVSLTC LVKGFYPSDI AVEWESNGQP ENNYKTTPPV
LDSDGSFFLY SRLTVDKSRW QEGNVFSCSV MHEALHNHYT QKSLSLSPG
Signal peptide: amino acids 1-20
Variable heavy chain: amino acids 25-143
CDR1: amino acids 50-56
CDR2: amino acids 76-81
CDR3: amino acids 123-132
IgG4 domain: amino acids 144-469
SEQ ID NO: 21 - Human IgG4 V_{L}
METDTLLLWV LLLWVPGSTG DIQMTQSPSS LSASVGDRVT ITCRSSQSIP SYLNWYQQKP
GKAPKLLIYA ASRLQSGVPS RFSGSGSGTD FTLTISSLQP EDFATYYCQQ SYSTPLTFGG
GTKVEIKRTV AAPSVFIFPP SDEQLKSGTA SVVCLLNNFY PREAKVQWKV DNALQSGNSQ
ESVTEQDSKD STYSLSSTLT LSKADYEKHK VYACEVTHQG LSSPVTKSFN RGEC
Leader sequence: amino acids 1-20
Variable light chain: amino acids 21-127
CDR1: amino acids 44-54
CDR2: amino acids 70-76
CDR3: amino acids 109-117
IgG4 domain: amino acids 128-234
SEQ ID NO: 22 - Canine IgG4 V_{L}
MRAWIFFLLC LAGRALAAPL ADIQMTQSPS SLSASVGDRV TITCRSSQSI PSTLNWYQQK
PGKAPKLLIY AASRLQSGVP SRFSGSGSGT DFTLTISSLQ PEDFATYYCQ QSYSTPLTFG
GGTKVEIKRT DAQPAVYLFQ PSPDQLHTGS ASVVCLLNSF YPKDINVKWK VDGVIQDTGI
QESVTEQDKD STYSLSSTLT MSSTEYLSHE LYSCEITHKS LPSTLIKSFQ RSECQRVD
Leader sequence: amino acids 1-21
Variable light chain: amino acids 22-128
CDR1: amino acids 45-55
CDR2: amino acids 71-77
CDR3: amino acids 110-118
Constant light chain κ: amino acids 129-238
SEQ ID NO: 23 - Canine IgG4 V_{H}
MRAWIFFLLC LAGRALAAPL AEVQLLESGG GLVQPGGSLR LSCAASGFTF SSYPMNWVRQ
APGKGLEWVS YISPFGGMTD YATSVKGRFT ISRDNSKNTL YLQMNSLRAE DTAVYYCARD
AMRGAEVDYW GQGTLVTVSS ASTTAPSVFP LAPSCGSTSG STVALACLVS GYFPEPVTVS
WNSGSLTSGV HTFPSVLQSS GLYSLSSTVT VPSSRWPSET FTCNVVHPAS NTKVDKPVPK
ESTCKCISPC PVPESLGGPS VFIFPPKPKD ILRITRTPEI TCVVLDLGRE DPEVQISWFV
DGKEVHTAKT QPREQQFNST YRVVSVLPIE HQDWLTGKEF KCRVNHIGLP SPIERTISKA
RGQAHQPSVY VLPPSPKELS SSDTVTLTCL IKDFFPPEID VEWQSNGQPE PESKYHTTAP
QLDEDGSYFL YSKLSVDKSR WQQGDTFTCA VMHEALQNHY TDLSLSHSPG KAAALEGS
Leader sequence: amino acids 1-21
Variable heavy domain: amino acids 22-140
CDR1: amino acids 47-53
CDR2: amino acids 73-78
CDR3: amino acids 120-129
Constant heavy domain: amino acids 141-478
SEQ ID NO: 24 - ACE2 V_{H}
EVQLLESGGG LVQPGGSLRL SCAASGFTFS SYPMNWVRQA PGKGLEWVSY ISPFGGMTDY
ATSVKGRFTI SRDNSKNTLY LQMNSLRAED TAVYYCARDA MRGAEVDYWG QGTLVTVSS
SEQ ID NO: 25 - ACE2 V_{L}
DIQMTQSPSS LSASVGDRVT ITCRSSQSIP SYLNWYQQKP GKAPKLLIYA ASRLQSGVPS
RFSGSGSGTD FTLTISSLQP EDFATYYCQQ SYSTPLTFGG GTKVEIK
Exemplary linkers
SEQ ID NO: 26
SGGGGSGGGG SGGGGSGGGG SG
SEQ ID NO: 27 - Whitlow linker
GSGKPGSGEG STKG
SEQ ID NO: 28
GGGGS
SEQ ID NO: 29
GGGGSGGGGS
SEQ ID NO: 30
GGGGSGGGGS GGGGS
SEQ ID NO: 31
EPKSSDKTHT SPPSP
SEQ ID NO: 32
RATPSHNSHQ VPSAGGPTAN SGTSG
SEQ ID NO: 33
SSGGGGSGGG GGGSSRSSL
SEQ ID NO: 34
PSGQAGAAAS ESLFVSNHAY
SEQ ID NO: 35
EASGGPE
SEQ ID NO: 36 - exemplary CD16-ACE2 TAB
MKWVTFISLL FLFSSAYSQV QLVESGGGLV QPGGSLRLSC AASGLTFSSY NMGWFRQAPG
QGLEAVASIT WSGRDTFYAD SVKGRFTISR DNSKNTLYLQ MNSLRAEDTA VYYCAANPWP
VAAPRSGTYW GQGTLVTVSS SGGGGSGGGG SGGGGSGGGG SGGSEVQLLE SGGGLVQPGG
SLRLSCAASG FTFSSYPMNW VRQAPGKGLE WVSYISPEGG MTDYATSVKG RFTISRDNSK
NTLYLQMNSL RAEDTAVYYC ARDAMRGAEV DYWGQGTLVT VSSGGGGSGG GGSGGGGSDI
QMTQSPSSLS ASVGDRVTIT CRSSQSIPSY LNWYQQKPGK APKLLIYAAS RLQSGVPSRF
SGSGSGTDFT LTISSLQPED FATYYCQQSY STPLTFGGGT KVEIKGS
Signal peptide (albumin): amino acids 1-18
hEF91 - camelid anti-CD16: amino acids 19-140
Linker: amino acids 141-164
anti-ACE2 V_{H}: amino acids 165-283
Linker: amino acids 284-298
anti-ACE2 VL: amino acids 299-405
SEQ ID NO: 37 - exemplary CD56/ADAM17 TAB
MKWVTFISLL FLFSSAYSEV QLVQSGAEVK KPGSSVKVSC KASGGTFTGY YMHWVRQAPG
QGLEWMGWIN PNSGGTNYAQ KFQGRVTMTR DTSISTAYME LSRLRSDDTA VYYCARDLSS
GYSGYFDYWG QGTLVTVSSG GGGSGGGGSG GGSDVVMTQS PLSLPVTPGE PASISCRSSQ
SLLHSNGYNY LDWYLQKPGQ SPQLLIYLGS NRASGVPDRF SGSGSGTDFT LKISRVEAED
VGVYYCMQAL QTPPYTFGQG TKLEIKRTVA GSTSGSGKPG SGEGSTKGGS EVQLLESGGG
LVQPGGSLRL SCAASGFTFS SYPMNWVRQA PGKGLEWVSY ISPFGGMTDY ATSVKGRFTI
SRDNSKNTLY LQMNSLRAED TAVYYCARDA MRGAEVDYWG QGTLVTVSSG GGGSGGGGSG
GGGSDIQMTQ SPSSLSASVG DRVTITCRSS QSIPSYLNWY QQKPGKAPKL LIYAASRLQS
GVPSRFSGSG SGTDFTLTIS SLQPEDFATY YCQQSYSTPL TFGGGTKVEI K
Signal peptide: amino acids 1-18
anti-CD56: amino acids 19-270
Whitlow Linker: amino acids 271-288
anti-ADAM V_{H}: amino acids 291-349
(GGGGS)₃ Linker: amino acids 350-364
anti-ADAM17 V_{L}: amino acids 365-471
SEQ ID NO: 38 - exemplary CD3/ADAM17 TAB
MKWVTFISLL FLFSSAYSQV QLQQSGAELA RPGASVKMSC KASGYTFTRY TMHWVKQRPG
QGLEWIGYIN PSRGYTNYNQ KFKDKATLTT DKSSSTAYMQ LSSLTSEDSA VYYCARYYDD
HYCLDYWGQG TTLTVSSGGG GSGGGGSGGG GSQIVLTQSP AIMSASPGEK VTMTCSASSS
VSYMNWYQQK SGTSPKRWIY DTSKLASGVP AHFRGSGSGT SYSLTISGME AEDAATYYCQ
QWSSNPFTFG SGTKLEINRG STSGSGKPGS GEGSTKGGSE VQLLESGGGL VQPGGSLRLS
CAASGFTFSS YPMNWVRQAP GKGLEWVSYI SPFGGMTDYA TSVKGRFTIS RDNSKNTLYL
QMNSLRAEDT AVYYCARDAM RGAEVDYWGQ GTLVTVSSGG GGSGGGGSGG GGSDIQMTQS
PSSLSASVGD RVTITCRSSQ SIPSYLNWYQ QKPGKAPKLL IYAASRLQSG VPSRFSGSGS
GTDFTLTISS LQPEDFATYY CQQSYSTPLT FGGGTKVEIK
Signal peptide: amino acids 1-18
anti-CD3 (OKT3): amino acids 19-259
Whitlow linker: amino acids 260-277
anti-ADAM17 V_{H}: amino acids 280-398
(GGGGS)₃ Linker: amino acids 399-413
anti-ADAM17 V_{L}: amino acids 414-520
SEQ ID NO: 39 - exemplary CD3/ADAM17 TAB
MKWVTFISLL FLFSSAYSEV QLQQSGPELV KPGASMKISC KASGYSFTGY TMNWVKQSHG
KNLEWMGLIN PYKGVSTYNQ KFKDKATLTV DKSSSTAYME LLSLTSEDSA VYYCARSGYY
GDSDWYFDVW GQGTTLTVFS GGGGSGGGGS GGGGSDIQMT QTTSSLSASL GDRVTISCRA
SQDIRNYLNW YQQKPDGTVK LLIYYTSRLH SGVPSKFSGS GSGTDYSLTI SNLEQEDIAT
YFCQQGNTLP WTFAGGTKLE IKGSTSGSGK PGSGEGSTKG GSEVQLLESG GGLVQPGGSL
RLSCAASGFT FSSYPMNWVR QAPGKGLEWV SYISPFGGMT DYATSVKGRF TISRDNSKNT
LYLQMNSLRA EDTAVYYCAR DAMRGAEVDY WGQGTLVTVS SGGGGSGGGG SGGGGSDIQM
TQSPSSLSAS VGDRVTITCR SSQSIPSYLN WYQQKPGKAP KLLIYAASRL QSGVPSRFSG
SGSGTDFTLT ISSLQPEDFA TYYCQQSYST PLTFGGGTKV EIK
Signal peptide: amino acids 1-18
anti-CD3 (UCHT): amino acids 19-262
Whitlow linker: amino acids 263-280
anti-ADAM17 V_{H}: amino acids 283-401
(GGGGS)₃ Linker: amino acids 402-416
anti-ADAM17 V_{L}: amino acids 417-523
SEQ ID NO: 40 - exemplary CD16/ADAM17 TAB
MKWVTFISLL FLFSSAYSQV QLVESGGGLV QPGGSLRLSC AASGLTFSSY NMGWFRQAPG
QGLEAVASIT WSGRDTFYAD SVKGRFTISR DNSKNTLYLQ MNSLRAEDTA VYYCAANPWP
VAAPRSGTYW GQGTLVTVSS SGGGGSGGGG SGGGGSGGGG SGGSEVQLLE SGGGLVQPGG
SLRLSCAASG FTFSSYPMNW VRQAPGKGLE WVSYISPFGG MIDYATSVKG RFTISRDNSK
NTLYLQMNSL RAEDTAVYYC ARDAMRGAEV DYWGQGTLVT VSSGGGGSGG GGSGGGGSDI
QMTQSPSSLS ASVGDRVTIT CRSSQSIPSY LNWYQQKPGK APKLLIYAAS RLQSGVPSRF
SGSGSGTDFT LTISSLQPED FATYYCQQSY STPLTFGGGT KVEIK
Signal peptide: amino acids 1-18
humanized camelid anti-CD16: amino acids 19-140
LinkerSeq16: amino acids 141-162
anti-ADAM17 V_{H}: amino acids 165-283
(GGGGS)₃ Linker: amino acids 284-298
anti-ADAM17 V_{L}: amino acids 299-405
SEQ ID NO: 41 - exemplary TMPRSS2/ADAM17 TAB
MKWVTFISLL FLFSSAYSQV QLVESGGGVV QPGRSLRLSC AASGFTFSSY GMHWVRQSPG
KGLEWVAVIW NDGSYVYYAD SVKGRFTISR DISKNTLFLQ MNSLRAEDTA VYYCAREGEW
VLYYFDYWGQ GTLVTVSSSG GGGSGGGGSG GGGSDIQMTQ SPSTLSASVG DRVTITCRAS
QSISSWLAWY QQKPGKAPKL LIYKASTLES GVPSRFSGSG SGTEFTLTIS SLQPDDFATY
YCQQYNSYSY TFGQGTKLEI KGSTSGSGKP GSGEGSTKGG SEVQLLESGG GLVQPGGSLR
LSCAASGFTF SSYPMNWVRQ APGKGLEWVS YISPFGGMTD YATSVKGRFT ISRDNSKNTL
YLQMNSLRAE DTAVYYCARD AMRGAEVDYW GQGTLVTVSS GGGGSGGGGS GGGGSDIQMT
QSPSSLSASV GDRVTITCRS SQSIPSYLNW YQQKPGKAPK LLIYAASRLQ SGVPSRFSGS
GSGTDFTLTI SSLQPEDFAT YYCQQSYSTP LTFGGGTKVE IK
Signal peptide: amino acids 1-18
anti-TMPRSS2: amino acids 19-261
Whitlow Linker: amino acids 262-279
anti-ADAM17 V_{H}: amino acids 282-400
(GGGGS)₃ Linker: amino acids 401-415
anti-ADAM17 V_{L}: amino acids 416-522
SEQ ID NO: 42 - exemplary ADAM17/B7H3 TAB
MKWVTFISLL FLFSSAYSEV QLLESGGGLV QPGGSLRLSC AASGFTFSSY PMNWVRQAPG
KGLEWVSYIS PFGGMTDYAT SVKGRFTISR DNSKNTLYLQ MNSLRAEDTA VYYCARDAMR
GAEVDYWGQG TLVTVSSGGG GSGGGGSGGG GSDIQMTQSP SSLSASVGDR VTITCRSSQS
IPSYLNWYQQ KPGKAPKLLI YAASRLQSGV PSRFSGSGSG TDFTLTISSL QPEDFATYYC
QQSYSTPLTF GGGTKVEIKS GGGGSGGGGS GGGGSGGGGS GGSQVQLVES GGGLVQPGGS
LRLSCAASGF TFSSYWMYWV RQTPGKGLEW VSTINRDGSA TWYADSVKGR FTISRDNAKN
TGYLQMNSLK PDDTAVYYCV SDPDNYSSDE MVPYWGQGTQ VTVSS
Signal peptide: amino acids 1-18
anti-ADAM17 V_{H}: amino acids 19-137
(GGGGS)₃ Linker: amino acids 138-152
anti-ADAM17 V_{L}: amino acids 153-259
LinkerSeq 16: amino acids 260-281
anti-B7H3 (clone 2): amino acids 284-405
SEQ ID NO: 43 - exemplary ADAM17/CD19 TAB
MKWVTFISLL FLFSSAYSEV QLLESGGGLV QPGGSLRLSC AASGFTFSSY PMNWVRQAPG
KGLEWVSYIS PFGGMTDYAT SVKGRFTISR DNSKNTLYLQ MNSLRAEDTA VYYCARDAMR
GAEVDYWGQG TLVTVSSGGG GSGGGGSGGG GSDIQMTQSP SSLSASVGDR VTITCRSSQS
IPSYLNWYQQ KPGKAPKLLI YAASRLQSGV PSRFSGSGSG TDFTLTISSL QPEDFATYYC
QQSYSTPLTF GGGTKVEIKS GGGGSGGGGS GGGGSGGGGS GGSDILLTQT PASLAVSLGQ
RATISCKASQ SVDYDGDSYL NWYQQIPGQP PKLLIYDASN LVSGIPPRFS GSGSGTDFTL
NIHPVEKVDA ATYHCQQSTE DPWTFGGGTK LEIKRGGGGS GGGGSGGGGS QVQLQQSGAE
LVRPGSSVKI SCKASGYAFS SYWMNWVKQR PGQGLEWIGQ IWPGDGDTNY NGKFKGKATL
TADESSSTAY MQLSSLASED SAVYFCARRE TTTVGRYYYA MDYWGQGTSV TVSS
Signal peptide: amino acids 1-18
anti-ADAM17 V_{H}: amino acids 19-137
(GGGGS)₃ Linker: amino acids 138-152
anti-ADAM17 V_{L}: amino acids 153-259
LinkerSeq 16: amino acids 260-281
anti-CD19_V_{L}: amino acids 284-395
(GGGGS)₃ Linker: amino acids 396-410
anti-CD19_V_{H}: amino acids 411-474
SEQ ID NO: 44 - exemplary SARS CoV2_RBD/ADAM17 TAB
MKWVTFISLL FLFSSAYSTN LCPFGEVFNA TRFASVYAWN RKRISNCVAD YSVLYNSASF
STFKCYGVSP TKLNDLCFTN VYADSFVIRG DEVRQIAPGQ TGKIADYNYK LPDDFTGCVI
AWNSNNLDSK VGGNYNYLYR LFRKSNLKPF ERDISTEIYQ AGSTPCNGVE GFNCYFPLQS
YGFQPTNGVG YQPYRVVVLS FELLHAPATV CGPKKGSTSG SGKPGSGEGS TKGGSEVQLL
ESGGGLVQPG GSLRLSCAAS GFTFSSYPMN WVRQAPGKGL EWVSYISPFG GMTDYATSVK
GRFTISRDNS KNTLYLQMNS LRAEDTAVYY CARDAMRGAE VDYWGLGTLV TVSSGGGGSG
GGGSGGGGSD IQMTQSPSSL SASVGDRVTI TCRSSQSIPS YLNWYQQKPG KAPKLLIYAA
SRLQSGVPSR FSGSGSGTDF TLTISSLPQE DFATYYCQQS YSTPLTFGGG TKVEIK
Signal peptide: amino acids 1-18
SARS-CoV2 RBD: amino acids 19-215
Whitlow linker: amino acids 216-233
anti-ADAM17 V_{H}: amino acids 235-354
(GGGGS)₃ Linker: amino acids 355-369
anti-ADAM17 V_{L}: amino acids 370-476
SEQ ID NO: 45 - polynucleotide encoding SEQ ID NO: 37
ATGAAGTGGG TAACCTTTAT TTCCCTTCTT TTTCTCTTTA GCTCGGCTTA TTCCGAGGTA
CAACTCGTCC AAAGTGGTGC GGAAGTGAAG AAGCCCGGGT CCTCTGTGAA AGTATCTTGT
AAGGCAAGTG GTGGTACCTT TACCGGATAT TACATGCATT GGGTTCGACA AGCACCGGGG
CAGGGATTGG AGTGGATGGG ATGGATCAAC CCGAACAGTG GGGGGACCAA CTATGCACAA
AAGTTTCAGG GGCGCGTTAC CATGACTCGG GACACCAGTA TATCTACGGC CTATATGGAA
CTTTCACGAC TCCGCAGTGA TGACACTGCA GTCTACTATT GCGCACGGGA TCTGTCATCC
GGCTATTCAG GGTATTTTGA CTATTGGGGG CAGGGTACAC TTGTAACAGT AAGCAGTGGT
GGCGGTGGTA GCGGCGGCGG CGGTTCCGGT GGCGGCAGTG ATGTTGTAAT GACACAGAGT
CCATTGAGCC TTCCAGTTAC TCCGGGCGAG CCAGCAAGCA TATCCTGCCG ATCTTCTCAA
TCATTGTTGC ACTCAAATGG CTATAACTAT CTCGATTGGT ATCTCCAGAA ACCCGGGCAA
TCCCCTCAAC TGCTCATTTA TCTGGGATCC AACAGGGCGT CTGGCGTGCC GGATAGGTTT
TCTGGTTCTG GATCAGGCAC GGACTTCACG TTGAAAATCA GTCGGGTCGA AGCAGAAGAC
GTGGGTGTCT ACTATTGCAT GCAGGCGCTC CAAACGCCGC CGTACACATT TGGACAGGGC
ACTAAACTTG AAATTAAGAG GACGGTGGCA GGTTCAACGA GCGGATCTGG AAAGCCGGGT
TCCGGGGAGG GCTCAACGAA GGGCGGTTCT GAAGTGCAGT TGTTAGAGTC AGGAGGCGGG
CTTGTTCAGC CTGGTGGTTC TCTGCGGCTT TCATGCGCTG CTTCCGGATT TACATTCTCT
TCCTACCCCA TGAATTGGGT TCGCCAAGCC CCTGGAAAAG GCTTGGAGTG GGTGTCCTAT
ATCTCTCCCT TCGGCGGCAT GACTGATTAT GCTACCAGCG TAAAGGGCCG GTTCACTATC
TCTAGAGACA ACTCGAAGAA CACGCTCTAC CTACAGATGA ACAGCCTGAG GGCCGAAGAC
ACGGCCGTGT ATTACTGTGC GAGAGATGCC ATGCGCGGGG CAGAGGTGGA CTACTGGGGC
CAGGGCACCC TGGTCACAGT ATCAAGCGGC GGAGGTGGGA GCGGTGGGGG GGGTAGCGGG
GGAGGCGGCT CTGACATTCA GATGACCCAG TCTCCATCCT CCCTGTCAGC ATCTGTGGGA
GACAGAGTCA CCATCACATG CCGATCCAGT CAGAGCATTC CCAGCTATCT GAATTGGTAT
CAGCAGAAGC CAGGGAAAGC CCCGAAGCTC CTGATTTATG CTGCATCCCG TTTACAATCC
GGGGTCCCAT CAAGGTTCAG CGGCAGTGGA TCGGGGACAG ATTTTACTCT CACCATAAGC
AGTCTGCAAC CTGAAGATTT TGCCACTTAC TACTGTCAAC AGAGTTACAG TACCCCCCTC
ACTTTTGGCG GAGGAACAAA GGTGGAGATC AAA
SEQ ID NO: 46 - polynucleotide encoding SEQ ID NO: 38
ATGAAGTGGG TAACCTTTAT TTCCCTTCTT TTTCTCTTTA GCTCGGCTTA TTCCCAAGTG
CAGCTGCAGC AGAGTGGAGC TGAGCTGGCA CGACCCGGCG CTTCCGTGAA AATGTCATGT
AAGGCGTCAG GGTATACCTT CACTCGTTAT ACTATGCACT GGGTGAAACA GCGGCCCGGG
CAAGGTCTTG AGTGGATCGG ATACATCAAC CCCTCTAGGG GGTACACTAA CTACAATCAG
AAGTTTAAGG ACAAGGCGAC CCTCACGACC GACAAGTCCT CTTCCACCGC TTACATGCAG
CTGTCTTCGT TAACATCCGA GGATTCTGCA GTCTACTATT GTGCCAGATA CTATGATGAC
CACTATTGCT TGGATTACTG GGGTCAGGGG ACAACGCTTA CCGTTAGCAG CGGGGGCGGA
GGTAGCGGCG GAGGAGGAAG TGGCGGCGGG GGTAGCCAGA TAGTGCTGAC ACAAAGTCCT
GCAATTATGT CTGCCAGCCC TGGTGAAAAA GTTACCATGA CATGCAGCGC CAGTTCCAGC
GTATCATATA TGAATTGGTA TCAACAGAAA AGTGGGACTT CCCCGAAAAG GTGGATTTAC
GACACCTCAA AGCTCGCCTC TGGAGTCCCA GCACATTTTC GCGGCTCGGG CTCAGGCACA
AGCTATTCTC TAACTATTAG CGGCATGGAG GCCGAAGATG CTGCCACTTA CTACTGCCAG
CAGTGGTCCA GTAATCCATT CACATTCGGC TCCGGGACCA AGCTGGAAAT CAACAGAGGT
TCAACGAGCG GATCTGGAAA GCCGGGTTCC GGGGAGGGCT CAACGAAGGG CGGTTCTGAA
GTGCAGTTGT TAGAGTCAGG AGGCGGGCTT GTTCAGCCTG GTGGTTCTCT GCGGCTTTCA
TGCGCTGCTT CCGGATTTAC ATTCTCTTCC TACCCCATGA ATTGGGTTCG CCAAGCCCCT
GGAAAAGGCT TGGAGTGGGT GTCCTATATC TCTCCCTTCG GCGGCATGAC TGATTATGCT
ACCAGCGTAA AGGGCCGGTT CACTATCTCT AGAGACAACT CGAAGAACAC GCTCTACCTA
CAGATGAACA GCCTGAGGGC CGAAGACACG GCCGTGTATT ACTGTGCGAG AGATGCCATG
CGCGGGGCAG AGGTGGACTA CTGGGGCCAG GGCACCCTGG TCACAGTATC AAGCGGCGGA
GGTGGGAGCG GTGGGGGGGG TAGCGGGGGA GGCGGCTCTG ACATTCAGAT GACCCAGTCT
CCATCCTCCC TGTCAGCATC TGTGGGAGAC AGAGTCACCA TCACATGCCG ATCCAGTCAG
AGCATTCCCA GCTATCTGAA TTGGTATCAG CAGAAGCCAG GGAAAGCCCC GAAGCTCCTG
ATTTATGCTG CATCCCGTTT ACAATCCGGG GTCCCATCAA GGTTCAGCGG CAGTGGATCG
GGGACAGATT TTACTCTCAC CATAAGCAGT CTGCAACCTG AAGATTTTGC CACTTACTAC
TGTCAACAGA GTTACAGTAC CCCCCTCACT TTTGGCGGAG GAACAAAGGT GGAGATCAAA
SEQ ID NO: 47 - polynucleotide encoding SEQ ID NO: 39
ATGAAGTGGG TAACCTTTAT TTCCCTTCTT TTTCTCTTTA GCTCGGCTTA TTCCGAAGTG
CAACTGCAGC AAAGCGGGCC CGAGCTGGTG AAGCCCGGAG CCTCCATGAA GATCAGTTGC
AAGGCCTCTG GGTACTCTTT TACCGGTTAC ACTATGAATT GGGTCAAACA GTCGCATGGC
AAGAACCTGG AGTGGATGGG CTTGATCAAT CCTTATAAGG GCGTATCCAC CTATAACCAG
AAATTCAAAG ATAAGGCAAC ACTAACAGTC GACAAATCGT CAAGTACCGC ATATATGGAG
TTATTAAGCT TGACTTCCGA GGACTCAGCT GTTTATTACT GTGCTAGGAG CGGATATTAT
GGAGATTCAG ACTGGTACTT CGACGTGTGG GGACAGGGCA CCACTCTGAC GGTTTTTTCT
GGCGGTGGAG GCAGCGGCGG CGGCGGGAGC GGCGGGGGGG GCAGCGATAT CCAGATGACT
CAGACAACTT CCTCTCTGTC TGCCTCACTT GGGGATAGAG TGACCATCAG CTGCCGAGCC
TCTCAAGACA TACGCAATTA CCTTAACTGG TACCAGCAAA AGCCAGATGG AACCGTCAAA
CTCCTGATAT ACTACACCAG CCGGCTGCAC AGTGGAGTGC CTTCCAAATT CTCCGGTTCA
GGGTCCGGAA CGGATTACAG TCTCACAATT AGTAACCTCG AACAGGAGGA CATTGCTACA
TATTTTTGTC AGCAGGGTAA TACCCTCCCA TGGACATTCG CGGGGGGTAC TAAGCTGGAA
ATTAAGGGTT CAACGAGCGG ATCTGGAAAG CCGGGTTCCG GGGAGGGCTC AACGAAGGGC
GGTTCTGAAG TGCAGTTGTT AGAGTCAGGA GGCGGGCTTG TTCAGCCTGG TGGTTCTCTG
CGGCTTTCAT GCGCTGCTTC CGGATTTACA TTCTCTTCCT ACCCCATGAA TTGGGTTCGC
CAAGCCCCTG GAAAAGGCTT GGAGTGGGTG TCCTATATCT CTCCCTTCGG CGGCATGACT
GATTATGCTA CCAGCGTAAA GGGCCGGTTC ACTATCTCTA GAGACAACTC GAAGAACACG
CTCTACCTAC AGATGAACAG CCTGAGGGCC GAAGACACGG CCGTGTATTA CTGTGCGAGA
GATGCCATGC GCGGGGCAGA GGTGGACTAC TGGGGCCAGG GCACCCTGGT CACAGTATCA
AGCGGCGGAG GTGGGAGCGG TGGGGGGGGT AGCGGGGGAG GCGGCTCTGA CATTCAGATG
ACCCAGTCTC CATCCTCCCT GTCAGCATCT GTGGGAGACA GAGTCACCAT CACATGCCGA
TCCAGTCAGA GCATTCCCAG CTATCTGAAT TGGTATCAGC AGAAGCCAGG GAAAGCCCCG
AAGCTCCTGA TTTATGCTGC ATCCCGTTTA CAATCCGGGG TCCCATCAAG GTTCAGCGGC
AGTGGATCGG GGACAGATTT TACTCTCACC ATAAGCAGTC TGCAACCTGA AGATTTTGCC
ACTTACTACT GTCAACAGAG TTACAGTACC CCCCTCACTT TTGGCGGAGG AACAAAGGTG
GAGATCAAA
SEQ ID NO: 48 - polynucleotide encoding SEQ ID NO: 40
ATGAAGTGGG TAACCTTTAT TTCCCTTCTT TTTCTCTTTA GCTCGGCTTA TTCCCAGGTG
CAGCTGGTGG AGTCTGGGGG AGGCTTGGTG CAGCCTGGGG GCTCTCTGAG ACTCTCCTGT
GCAGCCTCTG GCCTCACCTT CAGTAGCTAT AACATGGGCT GGTTCCGCCA GGCTCCAGGG
CAAGGCCTTG AGGCTGTAGC ATCTATTACC TGGAGTGGTC GGGACACATT CTATGCAGAC
TCCGTGAAGG GCCGATTCAC CATCTCCAGA GACAACTCCA AGAACACTCT CTATCTGCAA
ATGAACAGCC TGCGCGCGGA GGACACGGCC GTTTATTATT GTGCTGCAAA CCCCTGGCCA
GTGGCGGCGC CACGTAGTGG CACCTACTGG GGCCAAGGGA CCCTGGTCAC AGTCTCCTCA
TCTGGCGGCG GCGGTTCTGG TGGAGGAGGT AGTGGGGGGG GAGGAAGCGG AGGGGGTGGC
TCAGGGGGTT CTGAAGTGCA GTTGTTAGAG TCAGGAGGCG GGCTTGTTCA GCCTGGTGGT
TCTCTGCGGC TTTCATGCGC TGCTTCCGGA TTTACATTCT CTTCCTACCC CATGAATTGG
GTTCGCCAAG CCCCTGGAAA AGGCTTGGAG TGGGTGTCCT ATATCTCTCC CTTCGGCGGC
ATGACTGATT ATGCTACCAG CGTAAAGGGC CGGTTCACTA TCTCTAGAGA CAACTCGAAG
AACACGCTCT ACCTACAGAT GAACAGCCTG AGGGCCGAAG ACACGGCCGT GTATTACTGT
GCGAGAGATG CCATGCGCGG GGCAGAGGTG GACTACTGGG GCCAGGGCAC CCTGGTCACA
GTATCAAGCG GCGGAGGTGG GAGCGGTGGG GGGGGTAGCG GGGGAGGCGG CTCTGACATT
CAGATGACCC AGTCTCCATC CTCCCTGTCA GCATCTGTGG GAGACAGAGT CACCATCACA
TGCCGATCCA GTCAGAGCAT TCCCAGCTAT CTGAATTGGT ATCAGCAGAA GCCAGGGAAA
GCCCCGAAGC TCCTGATTTA TGCTGCATCC CGTTTACAAT CCGGGGTCCC ATCAAGGTTC
AGCGGCAGTG GATCGGGGAC AGATTTTACT CTCACCATAA GCAGTCTGCA ACCTGAAGAT
TTTGCCACTT ACTACTGTCA ACAGAGTTAC AGTACCCCCC TCACTTTTGG CGGAGGAACA
AAGGTGGAGA TCAAA
SEQ ID NO: 49 - polynucleotide encoding SEQ ID NO: 41
ATGAAGTGGG TAACCTTTAT TAGCCTTCTA TTTCTCTTTA GCTCCGCTTA TAGCCAAGTG
CAGCTCGTCG AATCCGGCGG CGGCGTGGTG CAACCGGGAC GAAGCCTGAG GCTTTCTTGT
GCCGCAAGCG GGTTTACCTT CTCCAGCTAC GGGATGCACT GGGTCCGGCA GTCTCCAGGG
AAGGGGCTGG AGTGGGTCGC CGTTATCTGG AATGATGGAT CCTACGTCTA CTACGCAGAT
TCTGTGAAGG GACGCTTTAC TATTAGCCGC GACATATCCA AAAATACCCT CTTCTTGCAG
ATGAACTCAC TCAGGGCCGA GGATACCGCC GTTTACTACT GCGCCCGGGA AGGCGAGTGG
GTACTGTACT ATTTTGACTA CTGGGGCCAG GGTACTCTTG TGACGGTTTC TAGTAGTGGG
GGGGGTGGGA GTGGCGGGGG TGGTAGCGGT GGCGGCGGCT CCGATATCCA GATGACCCAG
TCGCCTTCAA CACTGAGCGC TTCCGTGGGA GACAGAGTGA CTATTACATG TAGAGCGAGC
CAGTCTATTA GTTCCTGGCT CGCTTGGTAT CAACAGAAGC CCGGAAAAGC TCCAAAATTA
TTGATATATA AGGCATCAAC TCTGGAAAGT GGCGTACCCT CTCGTTTCTC AGGATCAGGC
TCCGGTACCG AGTTCACCCT GACAATCTCT AGCCTGCAGC CTGACGACTT CGCTACATAT
TATTGCCAAC AGTACAACTC GTATAGCTAT ACATTTGGAC AGGGAACGAA GCTAGAGATC
AAAGGCAGTA CCAGCGGGTC AGGGAAACCT GGCAGTGGGG AAGGTTCCAC AAAAGGTGGT
TCTGAAGTGC AGTTGTTAGA GTCAGGAGGC GGGCTTGTTC AGCCTGGTGG TTCTCTGCGG
CTTTCATGCG CTGCTTCCGG ATTTACATTC TCTTCCTACC CCATGAATTG GGTTCGCCAA
GCCCCTGGAA AAGGCTTGGA GTGGGTGTCC TATATCTCTC CCTTCGGCGG CATGACTGAT
TATGCTACCA GCGTAAAGGG CCGGTTCACT ATCTCTAGAG ACAACTCGAA GAACACGCTC
TACCTACAGA TGAACAGCCT GAGGGCCGAA GACACGGCCG TGTATTACTG TGCGAGAGAT
GCCATGCGCG GGGCAGAGGT GGACTACTGG GGCCAGGGCA CCCTGGTCAC AGTATCAAGC
GGCGGAGGTG GGAGCGGTGG GGGGGGTAGC GGGGGAGGCG GCTCTGACAT TCAGATGACC
CAGTCTCCAT CCTCCCTGTC AGCATCTGTG GGAGACAGAG TCACCATCAC ATGCCGATCC
AGTCAGAGCA TTCCCAGCTA TCTGAATTGG TATCAGCAGA AGCCAGGGAA AGCCCCGAAG
CTCCTGATTT ATGCTGCATC CCGTTTACAA TCCGGGGTCC CATCAAGGTT CAGCGGCAGT
GGATCGGGGA CAGATTTTAC TCTCACCATA AGCAGTCTGC AACCTGAAGA TTTTGCCACT
TACTACTGTC AACAGAGTTA CAGTACCCCC CTCACTTTTG GCGGAGGAAC AAAGGTGGAG
ATCAAA
SEQ ID NO: 50 - polynucleotide encoding SEQ ID NO: 42
ATGAAGTGGG TAACCTTTAT TTCCCTTCTT TTTCTCTTTA GCTCGGCTTA TTCCGAAGTG
CAGTTGTTAG AGTCAGGAGG CGGGCTTGTT CAGCCTGGTG GTTCTCTGCG GCTTTCATGC
GCTGCTTCCG GATTTACATT CTCTTCCTAC CCCATGAATT GGGTTCGCCA AGCCCCTGGA
AAAGGCTTGG AGTGGGTGTC CTATATCTCT CCCTTCGGCG GCATGACTGA TTATGCTACC
AGCGTAAAGG GCCGGTTCAC TATCTCTAGA GACAACTCGA AGAACACGCT CTACCTACAG
ATGAACAGCC TGAGGGCCGA AGACACGGCC GTGTATTACT GTGCGAGAGA TGCCATGCGC
GGGGCAGAGG TGGACTACTG GGGCCAGGGC ACCCTGGTCA CAGTATCAAG CGGTGGAGGT
GGTAGCGGTG GTGGTGGTAG CGGGGGAGGC GGCTCTGACA TTCAGATGAC CCAGTCTCCA
TCCTCCCTGT CAGCATCTGT GGGAGACAGA GTCACCATCA CATGCCGATC CAGTCAGAGC
ATTCCCAGCT ATCTGAATTG GTATCAGCAG AAGCCAGGGA AAGCCCCGAA GCTCCTGATT
TATGCTGCAT CCCGTTTACA ATCCGGGGTC CCATCAAGGT TCAGCGGCAG TGGATCGGGG
ACAGATTTTA CTCTCACCAT AAGCAGTCTG CAACCTGAAG ATTTTGCCAC TTACTACTGT
CAACAGAGTT ACAGTACCCC CCTCACTTTT GGCGGAGGAA CAAAGGTGGA GATCAAATCT
GGCGGCGGCG GTTCTGGTGG AGGAGGTAGT GGGGGGGGAG GAAGCGGAGG TGGTGGCTCA
GGGGGTTCTC AGGTTCAGCT GGTGGAGTCT GGCGGAGGCT TGGTGCAGCC CGGCGGGTCG
CTGAGACTCT CCTGCGCTGC CTCCGGATTC ACCTTTAGTA GCTACTGGAT GTACTGGGTT
AGGCAAACTC CAGGGAAAGG TCTTGAGTGG GTATCCACTA TTAATCGGGA TGGTAGTGCG
ACATGGTATG CAGACTCAGT GAAGGGCCGA TTCACCATCT CCCGCGACAA TGCCAAGAAC
ACAGGATATC TGCAAATGAA CAGCTTAAAA CCTGACGACA CGGCTGTGTA TTACTGTGTG
AGCGATCCGG ATAACTACTC TAGCGATGAA ATGGTCCCCT ATTGGGGCCA GGGGACCCAG
GTCACAGTCT CTTCA
SEQ ID NO: 51 - polynucleotide encoding SEQ ID NO: 43
ATGAAGTGGG TAACCTTTAT TTCCCTTCTT TTTCTCTTTA GCTCGGCTTA TTCCGAAGTG CAGTTGTTAG
AGTCAGGAGG CGGGCTTGTT CAGCCTGGTG GTTCTCTGCG GCTTTCATGC GCTGCTTCCG GATTTACATT
CTCTTCCTAC CCCATGAATT GGGTTCGCCA AGCCCCTGGA AAAGGCTTGG AGTGGGTGTC CTATATCTCT
CCCTTCGGCG GCATGACTGA TTATGCTACC AGCGTAAAGG GCCGGTTCAC TATCTCTAGA GACAACTCGA
AGAACACGCT CTACCTACAG ATGAACAGCC TGAGGGCCGA AGACACGGCC GTGTATTACT GTGCGAGAGA
TGCCATGCGC GGGGCAGAGG TGGACTACTG GGGCCAGGGC ACCCTGGTCA CAGTATCAAG CGGTGGAGGT
GGTAGCGGTG GTGGTGGTAG CGGGGGAGGC GGCTCTGACA TTCAGATGAC CCAGTCTCCA TCCTCCCTGT
CAGCATCTGT GGGAGACAGA GTCACCATCA CATGCCGATC CAGTCAGAGC ATTCCCAGCT ATCTGAATTG
GTATCAGCAG AAGCCAGGGA AAGCCCCGAA GCTCCTGATT TATGCTGCAT CCCGTTTACA ATCCGGGGTC
CCATCAAGGT TCAGCGGCAG TGGATCGGGG ACAGATTTTA CTCTCACCAT AAGCAGTCTG CAACCTGAAG
ATTTTGCCAC TTACTACTGT CAACAGAGTT ACAGTACCCC CCTCACTTTT GGCGGAGGAA CAAAGGTGGA
GATCAAATCT GGCGGCGGCG GTTCTGGTGG AGGAGGTAGT GGGGGGGGAG GAAGCGGAGG TGGTGGCTCA
GGGGGTTCTG ATATATTACT GACCCAAACT CCAGCTTCTT TGGCCGTGTC TCTGGGGCAG CGCGCCACCA
TCTCCTGCAA GGCCAGCCAA TCGGTTGATT ATGACGGTGA CAGTTACTTG AACTGGTACC AACAGATTCC
AGGACAGCCA CCCAAACTTC TCATCTATGA TGCATCCAAT CTGGTTTCAG GGATACCCCC CAGGTTTAGT
GGCAGTGGGT CTGGGACAGA CTTTACACTC AACATCCATC CCGTGGAGAA GGTGGATGCT GCGACCTATC
ACTGTCAGCA AAGTACTGAA GATCCGTGGA CGTTCGGAGG AGGCACCAAG CTGGAAATCA AACGAGGCGG
AGGTGGGAGC GGTGGGGGGG GTAGCGGGGG AGGCGGCTCT CAGGTGCAGC TGCAGCAGTC AGGGGCCGAG
CTGGTGCGGC CTGGGAGCTC AGTGAAGATT TCCTGCAAGG CTTCGGGCTA CGCATTCAGT AGCTACTGGA
TGAATTGGGT CAAGCAGAGG CCTGGACAGG GCCTTGAGTG GATTGGACAG ATCTGGCCTG GCGACGGTGA
TACTAACTAC AATGGAAAAT TTAAGGGCAA AGCCACACTG ACTGCAGACG AATCCTCCAG CACAGCCTAC
ATGCAGCTCA GCAGCCTAGC ATCCGAGGAC TCTGCGGTCT ATTTCTGTGC CAGACGGGAG ACTACAACGG
TAGGCAGATA TTACTATGCT ATGGACTACT GGGGTCAAGG AACCTCAGTC ACAGTATCTT CA
SEQ ID NO: 52 - polynucleotide encoding SEQ ID NO: 44
ATGAAGTGGG TAACCTTTAT TTCCCTTCTT TTTCTCTTTA GCTCGGCTTA TTCCGAAGTG
CAGTTGTTAG AGTCAGGAGG CGGGCTTGTT CAGCCTGGTG GTTCTCTGCG GCTTTCATGC
GCTGCTTCCG GATTTACATT CTCTTCCTAC CCCATGAATT GGGTTCGCCA AGCCCCTGGA
AAAGGCTTGG AGTGGGTGTC CTATATCTCT CCCTTCGGCG GCATGACTGA TTATGCTACC
AGCGTAAAGG GCCGGTTCAC TATCTCTAGA GACAACTCGA AGAACACGCT CTACCTACAG
ATGAACAGCC TGAGGGCCGA AGACACGGCC GTGTATTACT GTGCGAGAGA TGCCATGCGC
GGGGCAGAGG TGGACTACTG GGGCCAGGGC ACCCTGGTCA CAGTATCAAG CGGTGGAGGT
GGTAGCGGTG GTGGTGGTAG CGGGGGAGGC GGCTCTGACA TTCAGATGAC CCAGTCTCCA
TCCTCCCTGT CAGCATCTGT GGGAGACAGA GTCACCATCA CATGCCGATC CAGTCAGAGC
ATTCCCAGCT ATCTGAATTG GTATCAGCAG AAGCCAGGGA AAGCCCCGAA GCTCCTGATT
TATGCTGCAT CCCGTTTACA ATCCGGGGTC CCATCAAGGT TCAGCGGCAG TGGATCGGGG
ACAGATTTTA CTCTCACCAT AAGCAGTCTG CAACCTGAAG ATTTTGCCAC TTACTACTGT
CAACAGAGTT ACAGTACCCC CCTCACTTTT GGCGGAGGAA CAAAGGTGGA GATCAAATCT
GGCGGCGGCG GTTCTGGTGG AGGAGGTAGT GGGGGGGGAG GAAGCGGAGG TGGTGGCTCA
GGGGGTTCTG ATATATTACT GACCCAAACT CCAGCTTCTT TGGCCGTGTC TCTGGGGCAG
CGCGCCACCA TCTCCTGCAA GGCCAGCCAA TCGGTTGATT ATGACGGTGA CAGTTACTTG
AACTGGTACC AACAGATTCC AGGACAGCCA CCCAAACTTC TCATCTATGA TGCATCCAAT
CTGGTTTCAG GGATACCCCC CAGGTTTAGT GGCAGTGGGT CTGGGACAGA CTTTACACTC
AACATCCATC CCGTGGAGAA GGTGGATGCT GCGACCTATC ACTGTCAGCA AAGTACTGAA
GATCCGTGGA CGTTCGGAGG AGGCACCAAG CTGGAAATCA AACGAGGCGG AGGTGGGAGC
GGTGGGGGGG GTAGCGGGGG AGGCGGCTCT CAGGTGCAGC TGCAGCAGTC AGGGGCCGAG
CTGGTGCGGC CTGGGAGCTC AGTGAAGATT TCCTGCAAGG CTTCGGGCTA CGCATTCAGT
AGCTACTGGA TGAATTGGGT CAAGCAGAGG CCTGGACAGG GCCTTGAGTG GATTGGACAG
ATCTGGCCTG GCGACGGTGA TACTAACTAC AATGGAAAATT TAAGGGCAA AGCCACACTG
ACTGCAGACG AATCCTCCAG CACAGCCTAC ATGCAGCTCAG CAGCCTAGC ATCCGAGGAC
TCTGCGGTCT ATTTCTGTGC CAGACGGGAG ACTACAACGGT AGGCAGATA TTACTATGCT
ATGGACTACT GGGGTCAAGG AACCTCAGTC ACAGTATCTT CA
SEQ ID NO: 53
AAGAGTTACA CTGCCTTTGC CATC
SEQ ID NO: 54
CACTGTCTGC TGGTGGAGTT CATC
SEQ ID NO: 55
GGGTACCATG AAGATCTCTG CA
SEQ ID NO: 56
TTGGCGGTTC CTTCGAGTGA
SEQ ID NO: 57
TAGTCCTTCC TACCCCAATT TCC
SEQ ID NO: 58
TTGGTCCTTA GCCACTCCTT C
SEQ ID NO: 59
AAGCCTGTAG CCCACGTCGT A
SEQ ID NO: 60
GGCACCACTA GTTGGTTGTC TTTG
SEQ ID NO: 61
GCTCTTACTG ACTGGCATGA G
SEQ ID NO: 62
CGCAGCTCTA GGAGCATGTG
SEQ ID NO: 63 - anti-canine CD20 heavy chain
MGWSYIXXFL VATDVHSQVQ LQQPGAELVK PGASVKLSCK ASGYIFTTYW MNWVKQRPGQ
GLEWIGEISP SDGRINYNEK FKNKATLTVD KSSSTAYMQL SSLTSEDSAV YYCARDSYWG
QGTLVTVSAA KTTPPSVYPL XPGSLGRAN
X₇ = I or M
X₈ = L or F
X₁₄₁ = A or V
CDR1: amino acids 44-52
CDR2: amino acids 67-83
CDR3: amino acids 116-119
SEQ ID NO: 64
YIFTTYWMN
SEQ ID NO: 65
EISPSDGRIN YNEKFKN
SEQ ID NO: 66
DSYW
SEQ ID NO: 67 - anti-canine CD20 light chain
DXVMSQSPSS LAVSLGEKIT MSCKSSQSLL YSSNQKSYLA WYQQKPGQSP ELLIYWASTR
DXGVPDRFTG XGSGTDFTLT ISSVKAEDLA VXFCQQYYTY PYTFGGGTKL EIKRADAAPT
VSIFPPSSKL G
X₂ = I or T
X₆₂ = S or P
X₇₁ = G or S
X₉₂ = H or Y
CDR1: amino acids 24-40
CDR2: amino acids 56-62
CDR3: amino acids 95-103
SEQ ID NO: 68
KSSQSLLYSS NQKSYLA
SEQ ID NO: 69
WASTRDX
X₇ = S or P
SEQ ID NO: 70
QQYYTYPYT

1. A compound comprising:
a targeting domain that selectively binds to a target;
an ADAM17-inhibiting domain; and
a linker operably linking the targeting domain and the ADAM17-inhibiting domain.

2. The compound of claim 1, wherein the targeting domain selectively binds to an immune cell.

3. The compound of claim 2, wherein the immune cell is a T cell, a B cell, an NK cell, a monocyte, a macrophage, a dendritic cell, a myeloid-derived suppressor cell, or a mast cell.

4. The compound of claim 1, wherein the targeting domain comprises an antibody or a fragment thereof.

5. The compound of claim 4, wherein the antibody fragment comprises an scFv.

6. The compound of claim 4, wherein the antibody fragment comprises a single domain antibody.

7. (canceled)

8. (canceled)

9. The compound of claim 1, wherein the targeting domain selectively binds to a cell infected by a virus.

10. (canceled)

11. (canceled)

12. The compound of claim 1, wherein the targeting domain selectively binds to a cell infected by a bacterium.

13. (canceled)

14. The compound of claim 1, wherein the targeting domain selectively binds to a cell infected by a parasite.

15. (canceled)

16. The compound of claim 1, wherein the targeting domain selectively binds to a cell infected by a fungal cell.

17. (canceled)

18. The compound of claim 1, wherein the targeting domain selectively binds to a tumor cell.

19. A method of increasing NK cell proliferation, the method comprising:
contacting the compound of claim 1 with a population of NK cells under conditions effective for the NK cells to proliferate.

20. The method of claim 19, wherein the compound in contacted with the NK cells in vitro.

21. The method of claim 19, wherein the compound in contacted with the NK cells in vivo.

22-24. (canceled)

25. A human IgG4 antibody, or fragment thereof, comprising:
at least one of SEQ ID NO:17-19; and
amino acids 144-469 of SEQ ID NO:20.

26. (canceled)

27. A human IgG4 antibody, or fragment thereof, comprising:
at least one of SEQ ID NO:13-15; and
amino acids 128-234 of SEQ ID NO:21.

28. (canceled)

29. A therapeutic compound comprising:
an anti-ADAM17 domain comprising an antibody, or fragment thereof, comprising:
at least one of SEQ ID NO:17-19 and amino acids 144-469 of SEQ ID NO:20, or
at least one of SEQ ID NO:13-15 and amino acids 128-234 of SEQ ID NO:21; and
a targeting domain that specifically binds to a target of interest.

30-47. (canceled)


SEQUENCE LISTING (70 sequences)

SEQ ID NO: 1 - protein, 197 aa, SARS-CoV-2
TNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKK

SEQ ID NO: 2 - protein, 122 aa, Vicugna pacos
QVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSS

SEQ ID NO: 3 - protein, 254 aa, Artificial
  scFv
DILLTQTPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKRGSTSGSGKPGSGEGSTKGQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTSVTVSS

SEQ ID NO: 4 - protein, 247 aa, Artificial
  scFv
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSGSTSGSGKPGSGEGSTKGDIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIK

SEQ ID NO: 5 - protein, 248 aa, Artificial
  scFv
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGSTSGSGKPGSGEGSTKGDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTV

SEQ ID NO: 6 - protein, 247 aa, Artificial
  scFv
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAGSTSGSGKPGSGEGSTKGDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTV

SEQ ID NO: 7 - protein, 253 aa, Artificial
  Polypeptide
DIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELKRATPSHNSHQVPSAGGPTANSGTSGEVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSS

SEQ ID NO: 8 - protein, 122 aa, Artificial
  Polypeptide
HVQLVESGGGLVQPGRSLRLSCAASGFTFSSYWMYWVRQTPGKGLEWVSTINRDGSATWYADSVKGRFTISRDNAKNTGYLQMNSLEPDDTAVYYCVSDPDNYSSDEMVPYWGQGTQVTVSS

SEQ ID NO: 9 - protein, 122 aa, Artificial
  Polypeptide
QVQLVESGGGLVQPGGSLRLSCAASGFTFSSYWMYWVRQTPGKGLEWVSTINRDGSATWYADSVKGRFTISRDNAKNTGYLQMNSLKPDDTAVYYCVSDPDNYSSDEMVPYWGQGTQVTVSS

SEQ ID NO: 10 - protein, 241 aa, Artificial
  Polypeptide
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINR

SEQ ID NO: 11 - protein, 244 aa, Artificial
  Polypeptide
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIK

SEQ ID NO: 12 - protein, 107 aa, Artificial
  Polypeptide
  CDR1 (24)..(34)
  CDR2 (50)..(56)
  CDR3 (89)..(97)
DIQMTQSPSSLSASVGDRVTITCRSSQSIPSYLNWYQQKPGKAPKLLIYAASRLQSGVPSRFSGSGSGTDFTLTISSLQPEDFATYYCQQSYSTPLTFGGGTKVEIK

SEQ ID NO: 13 - protein, 11 aa, Artificial
  Polypeptide
RSSQSIPSYLN

SEQ ID NO: 14 - protein, 7 aa, Artificial
  Polypeptide
AASRLQS

SEQ ID NO: 15 - protein, 9 aa, Artificial
  Polypeptide
QQSYSTPLT

SEQ ID NO: 16 - protein, 119 aa, Artificial
  Polypeptide
  CDR1 (26)..(32)
  CDR2 (52)..(57)
  CDR3 (99)..(108)
EVQLLESGGGLVQPGGSLRLSCAASGFTFSSYPMNWVRQAPGKGLEWVSYISPFGGMTDYATSVKGRFTISRDNSKNTLYLQMNSLRAEDTAVYYCARDAMRGAEVDYWGQGTLVTVSS

SEQ ID NO: 17 - protein, 7 aa, Artificial
  Polypeptide
GFTFSSY

SEQ ID NO: 18 - protein, 6 aa, Artificial
  Polypeptide
SPFGGM

SEQ ID NO: 19 - protein, 10 aa, Artificial
  Polypeptide
DAMRGAEVDY

SEQ ID NO: 20 - protein, 469 aa, Homo sapiens
  Signal peptide (1)..(20)
  Variable heavy chain (25)..(143)
  CDR1 (50)..(56)
  CDR2 (76)..(81)
  CDR3 (123)..(132)
  IgG4 domain (144)..(469)
MDPKGSLSWRILLFLSLAFELSYGEVQLLESGGGLVQPGGSLRLSCAASGFTFSSYPMNWVRQAPGKGLEWVSYISPFGGMTDYATSVKGRFTISRDNSKNTLYLQMNSLRAEDTAVYYCARDAMRGAEVDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPG

SEQ ID NO: 21 - protein, 234 aa, Homo sapiens
  Leader sequence (1)..(20)
  Variable light chain (21)..(127)
  CDR1 (44)..(54)
  CDR2 (70)..(76)
  CDR3 (109)..(117)
  IgG4 domain (128)..(234)
METDTLLLWVLLLWVPGSTGDIQMTQSPSSLSASVGDRVTITCRSSQSIPSYLNWYQQKPGKAPKLLIYAASRLQSGVPSRFSGSGSGTDFTLTISSLQPEDFATYYCQQSYSTPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 238 aa, Canis familiaris
  Leader sequence (1)..(21)
  Variable light chain (22)..(128)
  CDR1 (45)..(55)
  CDR2 (71)..(77)
  CDR3 (110)..(118)
  Constant light chain kappa (129)..(238)
MRAWIFFLLCLAGRALAAPLADIQMTQSPSSLSASVGDRVTITCRSSQSIPSTLNWYQQKPGKAPKLLIYAASRLQSGVPSRFSGSGSGTDFTLTISSLQPEDFATYYCQQSYSTPLTFGGGTKVEIKRTDAQPAVYLFQPSPDQLHTGSASVVCLLNSFYPKDINVKWKVDGVIQDTGIQESVTEQDKDSTYSLSSTLTMSSTEYLSHELYSCEITHKSLPSTLIKSFQRSECQRVD

SEQ ID NO: 23 - protein, 478 aa, Canis familiaris
  Leader sequence (1)..(21)
  Variable heavy domain (22)..(140)
  CDR1 (47)..(53)
  CDR2 (73)..(78)
  CDR3 (120)..(129)
  Constant heavy domain (141)..(478)
MRAWIFFLLCLAGRALAAPLAEVQLLESGGGLVQPGGSLRLSCAASGFTFSSYPMNWVRQAPGKGLEWVSYISPFGGMTDYATSVKGRFTISRDNSKNTLYLQMNSLRAEDTAVYYCARDAMRGAEVDYWGQGTLVTVSSASTTAPSVFPLAPSCGSTSGSTVALACLVSGYFPEPVTVSWNSGSLTSGVHTFPSVLQSSGLYSLSSTVTVPSSRWPSETFTCNVVHPASNTKVDKPVPKESTCKCISPCPVPESLGGPSVFIFPPKPKDILRITRTPEITCVVLDLGREDPEVQISWFVDGKEVHTAKTQPREQQFNSTYRVVSVLPIEHQDWLTGKEFKCRVNHIGLPSPIERTISKARGQAHQPSVYVLPPSPKELSSSDTVTLTCLIKDFFPPEIDVEWQSNGQPEPESKYHTTAPQLDEDGSYFLYSKLSVDKSRWQQGDTFTCAVMHEALQNHYTDLSLSHSPGKAAALEGS

SEQ ID NO: 24 - protein, 119 aa, Artificial; identical to SEQ ID NO: 16
  Polypeptide

SEQ ID NO: 25 - protein, 107 aa, Artificial; identical to SEQ ID NO: 12
  Polypeptide

SEQ ID NO: 26 - protein, 22 aa, Artificial
  linker
SGGGGSGGGGSGGGGSGGGGSG

SEQ ID NO: 27 - protein, 14 aa, Artificial
  linker
GSGKPGSGEGSTKG

SEQ ID NO: 28 - protein, 5 aa, Artificial
  linker
GGGGS

SEQ ID NO: 29 - protein, 10 aa, Artificial
  linker
GGGGSGGGGS

SEQ ID NO: 30 - protein, 15 aa, Artificial
  linker
GGGGSGGGGSGGGGS

SEQ ID NO: 31 - protein, 15 aa, Artificial
  linker
EPKSSDKTHTSPPSP

SEQ ID NO: 32 - protein, 25 aa, Artificial
  linker
RATPSHNSHQVPSAGGPTANSGTSG

SEQ ID NO: 33 - protein, 19 aa, Artificial
  linker
SSGGGGSGGGGGGSSRSSL

SEQ ID NO: 34 - protein, 20 aa, Artificial
  linker
PSGQAGAAASESLFVSNHAY

SEQ ID NO: 35 - protein, 7 aa, Artificial
  linker
EASGGPE

SEQ ID NO: 36 - protein, 407 aa, Artificial
  Polypeptide
  Signal peptide (albumin) (1)..(18)
  huEF91 - camelid anti-CD16 (19)..(140)
  Linker (141)..(164)
  anti-ACE2 VH (165)..(283)
  Linker (284)..(298)
  anti-ACE2 VL (299)..(405)
MKWVTFISLLFLFSSAYSQVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSSSGGGGSGGGGSGGGGSGGGGSGGSEVQLLESGGGLVQPGGSLRLSCAASGFTFSSYPMNWVRQAPGKGLEWVSYISPFGGMTDYATSVKGRFTISRDNSKNTLYLQMNSLRAEDTAVYYCARDAMRGAEVDYWGQGTLVTVSSGGGGSGGGGSGGGGSDIQMTQSPSSLSASVGDRVTITCRSSQSIPSYLNWYQQKPGKAPKLLIYAASRLQSGVPSRFSGSGSGTDFTLTISSLQPEDFATYYCQQSYSTPLTFGGGTKVEIKGS

SEQ ID NO: 37 - protein, 531 aa, Artificial
  Polypeptide
  Signal peptide (1)..(18)
  anti-CD56 (19)..(270)
  Whitlow Linker (271)..(288)
  anti-ADAM VH (291)..(349)
  (GGGGS)3 Linker (350)..(364)
  anti-ADAM17 VL (365)..(471)
MKWVTFISLLFLFSSAYSEVQLVQSGAEVKKPGSSVKVSCKASGGTFTGYYMHWVRQAPGQGLEWMGWINPNSGGTNYAQKFQGRVTMTRDTSISTAYMELSRLRSDDTAVYYCARDLSSGYSGYFDYWGQGTLVTVSSGGGGSGGGGSGGGSDVVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPPYTFGQGTKLEIKRTVAGSTSGSGKPGSGEGSTKGGSEVQLLESGGGLVQPGGSLRLSCAASGFTFSSYPMNWVRQAPGKGLEWVSYISPFGGMTDYATSVKGRFTISRDNSKNTLYLQMNSLRAEDTAVYYCARDAMRGAEVDYWGQGTLVTVSSGGGGSGGGGSGGGGSDIQMTQSPSSLSASVGDRVTITCRSSQSIPSYLNWYQQKPGKAPKLLIYAASRLQSGVPSRFSGSGSGTDFTLTISSLQPEDFATYYCQQSYSTPLTFGGGTKVEIK

SEQ ID NO: 38 - protein, 520 aa, Artificial
  Polypeptide
  Signal peptide (1)..(18)
  anti-CD3 (OKT3) (19)..(259)
  linker (260)..(277)
  anti-ADAM17 VH (280)..(398)
  linker (399)..(413)
  anti-ADAM17 VL (414)..(520)
MKWVTFISLLFLFSSAYSQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSGGGGSGGGGSGGGGSQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRGSTSGSGKPGSGEGSTKGGSEVQLLESGGGLVQPGGSLRLSCAASGFTFSSYPMNWVRQAPGKGLEWVSYISPFGGMTDYATSVKGRFTISRDNSKNTLYLQMNSLRAEDTAVYYCARDAMRGAEVDYWGQGTLVTVSSGGGGSGGGGSGGGGSDIQMTQSPSSLSASVGDRVTITCRSSQSIPSYLNWYQQKPGKAPKLLIYAASRLQSGVPSRFSGSGSGTDFTLTISSLQPEDFATYYCQQSYSTPLTFGGGTKVEIK

SEQ ID NO: 39 - protein, 523 aa, Artificial
  Polypeptide
  Signal peptide (1)..(18)
  anti-CD3 (UCHT) (19)..(262)
  linker (263)..(280)
  anti-ADAM17 VH (283)..(401)
  linker (402)..(416)
  anti-ADAM17 VL (417)..(523)
MKWVTFISLLFLFSSAYSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSGGGGSGGGGSGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKGSTSGSGKPGSGEGSTKGGSEVQLLESGGGLVQPGGSLRLSCAASGFTFSSYPMNWVRQAPGKGLEWVSYISPFGGMTDYATSVKGRFTISRDNSKNTLYLQMNSLRAEDTAVYYCARDAMRGAEVDYWGQGTLVTVSSGGGGSGGGGSGGGGSDIQMTQSPSSLSASVGDRVTITCRSSQSIPSYLNWYQQKPGKAPKLLIYAASRLQSGVPSRFSGSGSGTDFTLTISSLQPEDFATYYCQQSYSTPLTFGGGTKVEIK

SEQ ID NO: 40 - protein, 405 aa, Artificial
  Polypeptide
  Signal peptide (1)..(18)
  humanized camelid anti-CD16 (19)..(140)
  linker (141)..(162)
  anti-ADAM17 VH (165)..(283)
  linker (284)..(298)
  anti-ADAM17 VL (299)..(405)
MKWVTFISLLFLFSSAYSQVQLVESGGGLVQPGGSLRLSCAASGLTFSSYNMGWFRQAPGQGLEAVASITWSGRDTFYADSVKGRFTISRDNSKNTLYLQMNSLRAEDTAVYYCAANPWPVAAPRSGTYWGQGTLVTVSSSGGGGSGGGGSGGGGSGGGGSGGSEVQLLESGGGLVQPGGSLRLSCAASGFTFSSYPMNWVRQAPGKGLEWVSYISPFGGMTDYATSVKGRFTISRDNSKNTLYLQMNSLRAEDTAVYYCARDAMRGAEVDYWGQGTLVTVSSGGGGSGGGGSGGGGSDIQMTQSPSSLSASVGDRVTITCRSSQSIPSYLNWYQQKPGKAPKLLIYAASRLQSGVPSRFSGSGSGTDFTLTISSLQPEDFATYYCQQSYSTPLTFGGGTKVEIK

SEQ ID NO: 41 - protein, 522 aa, Artificial
  Polypeptide
  Signal peptide (1)..(18)
  anti-TMPRSS2 (19)..(261)
  linker (262)..(279)
  anti-ADAM17 VH (282)..(400)
  linker (401)..(415)
  anti-ADAM17 VL (416)..(522)
MKWVTFISLLFLFSSAYSQVQLVESGGGVVQPGRSLRLSCAASGFTFSSYGMHWVRQSPGKGLEWVAVIWNDGSYVYYADSVKGRFTISRDISKNTLFLQMNSLRAEDTAVYYCAREGEWVLYYFDYWGQGTLVTVSSSGGGGSGGGGSGGGGSDIQMTQSPSTLSASVGDRVTITCRASQSISSWLAWYQQKPGKAPKLLIYKASTLESGVPSRFSGSGSGTEFTLTISSLQPDDFATYYCQQYNSYSYTFGQGTKLEIKGSTSGSGKPGSGEGSTKGGSEVQLLESGGGLVQPGGSLRLSCAASGFTFSSYPMNWVRQAPGKGLEWVSYISPFGGMTDYATSVKGRFTISRDNSKNTLYLQMNSLRAEDTAVYYCARDAMRGAEVDYWGQGTLVTVSSGGGGSGGGGSGGGGSDIQMTQSPSSLSASVGDRVTITCRSSQSIPSYLNWYQQKPGKAPKLLIYAASRLQSGVPSRFSGSGSGTDFTLTISSLQPEDFATYYCQQSYSTPLTFGGGTKVEIK

SEQ ID NO: 42 - protein, 405 aa, Artificial
  Polypeptide
  Signal peptide (1)..(18)
  anti-ADAM17 VH (19)..(137)
  linker (138)..(152)
  anti-ADAM17 VL (153)..(259)
  linker (260)..(281)
  anti-B7H3 (284)..(405)
MKWVTFISLLFLFSSAYSEVQLLESGGGLVQPGGSLRLSCAASGFTFSSYPMNWVRQAPGKGLEWVSYISPFGGMTDYATSVKGRFTISRDNSKNTLYLQMNSLRAEDTAVYYCARDAMRGAEVDYWGQGTLVTVSSGGGGSGGGGSGGGGSDIQMTQSPSSLSASVGDRVTITCRSSQSIPSYLNWYQQKPGKAPKLLIYAASRLQSGVPSRFSGSGSGTDFTLTISSLQPEDFATYYCQQSYSTPLTFGGGTKVEIKSGGGGSGGGGSGGGGSGGGGSGGSQVQLVESGGGLVQPGGSLRLSCAASGFTFSSYWMYWVRQTPGKGLEWVSTINRDGSATWYADSVKGRFTISRDNAKNTGYLQMNSLKPDDTAVYYCVSDPDNYSSDEMVPYWGQGTQVTVSS

SEQ ID NO: 43 - protein, 534 aa, Artificial
  Polypeptide
  Signal peptide (1)..(18)
  anti-ADAM17 VH (19)..(137)
  linker (138)..(152)
  anti-ADAM17 VL (153)..(259)
  linker (260)..(281)
  anti-CD19_VL (284)..(395)
  linker (396)..(410)
  anti-CD19_VH (411)..(474)
MKWVTFISLLFLFSSAYSEVQLLESGGGLVQPGGSLRLSCAASGFTFSSYPMNWVRQAPGKGLEWVSYISPFGGMTDYATSVKGRFTISRDNSKNTLYLQMNSLRAEDTAVYYCARDAMRGAEVDYWGQGTLVTVSSGGGGSGGGGSGGGGSDIQMTQSPSSLSASVGDRVTITCRSSQSIPSYLNWYQQKPGKAPKLLIYAASRLQSGVPSRFSGSGSGTDFTLTISSLQPEDFATYYCQQSYSTPLTFGGGTKVEIKSGGGGSGGGGSGGGGSGGGGSGGSDILLTQTPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIKRGGGGSGGGGSGGGGSQVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTSVTVSS

SEQ ID NO: 44 - protein, 476 aa, Artificial
  Polypeptide
  Signal peptide (1)..(18)
  SARS-CoV2 RBD (19)..(215)
  linker (216)..(233)
  anti-ADAM17 VH (235)..(354)
  linker (355)..(369)
  anti-ADAM17 VL (370)..(476)
MKWVTFISLLFLFSSAYSTNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKGSTSGSGKPGSGEGSTKGGSEVQLLESGGGLVQPGGSLRLSCAASGFTFSSYPMNWVRQAPGKGLEWVSYISPFGGMTDYATSVKGRFTISRDNSKNTLYLQMNSLRAEDTAVYYCARDAMRGAEVDYWGQGTLVTVSSGGGGSGGGGSGGGGSDIQMTQSPSSLSASVGDRVTITCRSSQSIPSYLNWYQQKPGKAPKLLIYAASRLQSGVPSRFSGSGSGTDFTLTISSLQPEDFATYYCQQSYSTPLTFGGGTKVEIK

SEQ ID NO: 45 - DNA, 1593 bp, Artificial
  Polynucleotide
atgaagtgggtaacctttatttcccttctttttctctttagctcggcttattccgaggtacaactcgtccaaagtggtgcggaagtgaagaagcccgggtcctctgtgaaagtatcttgtaaggcaagtggtggtacctttaccggatattacatgcattgggttcgacaagcaccggggcagggattggagtggatgggatggatcaacccgaacagtggggggaccaactatgcacaaaagtttcaggggcgcgttaccatgactcgggacaccagtatatctacggcctatatggaactttcacgactccgcagtgatgacactgcagtctactattgcgcacgggatctgtcatccggctattcagggtattttgactattgggggcagggtacacttgtaacagtaagcagtggtggcggtggtagcggcggcggcggttccggtggcggcagtgatgttgtaatgacacagagtccattgagccttccagttactccgggcgagccagcaagcatatcctgccgatcttctcaatcattgttgcactcaaatggctataactatctcgattggtatctccagaaacccgggcaatcccctcaactgctcatttatctgggatccaacagggcgtctggcgtgccggataggttttctggttctggatcaggcacggacttcacgttgaaaatcagtcgggtcgaagcagaagacgtgggtgtctactattgcatgcaggcgctccaaacgccgccgtacacatttggacagggcactaaacttgaaattaagaggacggtggcaggttcaacgagcggatctggaaagccgggttccggggagggctcaacgaagggcggttctgaagtgcagttgttagagtcaggaggcgggcttgttcagcctggtggttctctgcggctttcatgcgctgcttccggatttacattctcttcctaccccatgaattgggttcgccaagcccctggaaaaggcttggagtgggtgtcctatatctctcccttcggcggcatgactgattatgctaccagcgtaaagggccggttcactatctctagagacaactcgaagaacacgctctacctacagatgaacagcctgagggccgaagacacggccgtgtattactgtgcgagagatgccatgcgcggggcagaggtggactactggggccagggcaccctggtcacagtatcaagcggcggaggtgggagcggtggggggggtagcgggggaggcggctctgacattcagatgacccagtctccatcctccctgtcagcatctgtgggagacagagtcaccatcacatgccgatccagtcagagcattcccagctatctgaattggtatcagcagaagccagggaaagccccgaagctcctgatttatgctgcatcccgtttacaatccggggtcccatcaaggttcagcggcagtggatcggggacagattttactctcaccataagcagtctgcaacctgaagattttgccacttactactgtcaacagagttacagtacccccctcacttttggcggaggaacaaaggtggagatcaaa

SEQ ID NO: 46 - DNA, 1560 bp, Artificial
  Polynucleotide
atgaagtgggtaacctttatttcccttctttttctctttagctcggcttattcccaagtgcagctgcagcagagtggagctgagctggcacgacccggcgcttccgtgaaaatgtcatgtaaggcgtcagggtataccttcactcgttatactatgcactgggtgaaacagcggcccgggcaaggtcttgagtggatcggatacatcaacccctctagggggtacactaactacaatcagaagtttaaggacaaggcgaccctcacgaccgacaagtcctcttccaccgcttacatgcagctgtcttcgttaacatccgaggattctgcagtctactattgtgccagatactatgatgaccactattgcttggattactggggtcaggggacaacgcttaccgttagcagcgggggcggaggtagcggcggaggaggaagtggcggcgggggtagccagatagtgctgacacaaagtcctgcaattatgtctgccagccctggtgaaaaagttaccatgacatgcagcgccagttccagcgtatcatatatgaattggtatcaacagaaaagtgggacttccccgaaaaggtggatttacgacacctcaaagctcgcctctggagtcccagcacattttcgcggctcgggctcaggcacaagctattctctaactattagcggcatggaggccgaagatgctgccacttactactgccagcagtggtccagtaatccattcacattcggctccgggaccaagctggaaatcaacagaggttcaacgagcggatctggaaagccgggttccggggagggctcaacgaagggcggttctgaagtgcagttgttagagtcaggaggcgggcttgttcagcctggtggttctctgcggctttcatgcgctgcttccggatttacattctcttcctaccccatgaattgggttcgccaagcccctggaaaaggcttggagtgggtgtcctatatctctcccttcggcggcatgactgattatgctaccagcgtaaagggccggttcactatctctagagacaactcgaagaacacgctctacctacagatgaacagcctgagggccgaagacacggccgtgtattactgtgcgagagatgccatgcgcggggcagaggtggactactggggccagggcaccctggtcacagtatcaagcggcggaggtgggagcggtggggggggtagcgggggaggcggctctgacattcagatgacccagtctccatcctccctgtcagcatctgtgggagacagagtcaccatcacatgccgatccagtcagagcattcccagctatctgaattggtatcagcagaagccagggaaagccccgaagctcctgatttatgctgcatcccgtttacaatccggggtcccatcaaggttcagcggcagtggatcggggacagattttactctcaccataagcagtctgcaacctgaagattttgccacttactactgtcaacagagttacagtacccccctcacttttggcggaggaacaaaggtggagatcaaa

SEQ ID NO: 47 - DNA, 1569 bp, Artificial
  Polynucleotide
atgaagtgggtaacctttatttcccttctttttctctttagctcggcttattccgaagtgcaactgcagcaaagcgggcccgagctggtgaagcccggagcctccatgaagatcagttgcaaggcctctgggtactcttttaccggttacactatgaattgggtcaaacagtcgcatggcaagaacctggagtggatgggcttgatcaatccttataagggcgtatccacctataaccagaaattcaaagataaggcaacactaacagtcgacaaatcgtcaagtaccgcatatatggagttattaagcttgacttccgaggactcagctgtttattactgtgctaggagcggatattatggagattcagactggtacttcgacgtgtggggacagggcaccactctgacggttttttctggcggtggaggcagcggcggcggcgggagcggcggggggggcagcgatatccagatgactcagacaacttcctctctgtctgcctcacttggggatagagtgaccatcagctgccgagcctctcaagacatacgcaattaccttaactggtaccagcaaaagccagatggaaccgtcaaactcctgatatactacaccagccggctgcacagtggagtgccttccaaattctccggttcagggtccggaacggattacagtctcacaattagtaacctcgaacaggaggacattgctacatatttttgtcagcagggtaataccctcccatggacattcgcggggggtactaagctggaaattaagggttcaacgagcggatctggaaagccgggttccggggagggctcaacgaagggcggttctgaagtgcagttgttagagtcaggaggcgggcttgttcagcctggtggttctctgcggctttcatgcgctgcttccggatttacattctcttcctaccccatgaattgggttcgccaagcccctggaaaaggcttggagtgggtgtcctatatctctcccttcggcggcatgactgattatgctaccagcgtaaagggccggttcactatctctagagacaactcgaagaacacgctctacctacagatgaacagcctgagggccgaagacacggccgtgtattactgtgcgagagatgccatgcgcggggcagaggtggactactggggccagggcaccctggtcacagtatcaagcggcggaggtgggagcggtggggggggtagcgggggaggcggctctgacattcagatgacccagtctccatcctccctgtcagcatctgtgggagacagagtcaccatcacatgccgatccagtcagagcattcccagctatctgaattggtatcagcagaagccagggaaagccccgaagctcctgatttatgctgcatcccgtttacaatccggggtcccatcaaggttcagcggcagtggatcggggacagattttactctcaccataagcagtctgcaacctgaagattttgccacttactactgtcaacagagttacagtacccccctcacttttggcggaggaacaaaggtggagatcaaa

SEQ ID NO: 48 - DNA, 1215 bp, Artificial
  Polynucleotide
atgaagtgggtaacctttatttcccttctttttctctttagctcggcttattcccaggtgcagctggtggagtctgggggaggcttggtgcagcctgggggctctctgagactctcctgtgcagcctctggcctcaccttcagtagctataacatgggctggttccgccaggctccagggcaaggccttgaggctgtagcatctattacctggagtggtcgggacacattctatgcagactccgtgaagggccgattcaccatctccagagacaactccaagaacactctctatctgcaaatgaacagcctgcgcgcggaggacacggccgtttattattgtgctgcaaacccctggccagtggcggcgccacgtagtggcacctactggggccaagggaccctggtcacagtctcctcatctggcggcggcggttctggtggaggaggtagtggggggggaggaagcggagggggtggctcagggggttctgaagtgcagttgttagagtcaggaggcgggcttgttcagcctggtggttctctgcggctttcatgcgctgcttccggatttacattctcttcctaccccatgaattgggttcgccaagcccctggaaaaggcttggagtgggtgtcctatatctctcccttcggcggcatgactgattatgctaccagcgtaaagggccggttcactatctctagagacaactcgaagaacacgctctacctacagatgaacagcctgagggccgaagacacggccgtgtattactgtgcgagagatgccatgcgcggggcagaggtggactactggggccagggcaccctggtcacagtatcaagcggcggaggtgggagcggtggggggggtagcgggggaggcggctctgacattcagatgacccagtctccatcctccctgtcagcatctgtgggagacagagtcaccatcacatgccgatccagtcagagcattcccagctatctgaattggtatcagcagaagccagggaaagccccgaagctcctgatttatgctgcatcccgtttacaatccggggtcccatcaaggttcagcggcagtggatcggggacagattttactctcaccataagcagtctgcaacctgaagattttgccacttactactgtcaacagagttacagtacccccctcacttttggcggaggaacaaaggtggagatcaaa

SEQ ID NO: 49 - DNA, 1566 bp, Artificial
  Polynucleotide
atgaagtgggtaacctttattagccttctatttctctttagctccgcttatagccaagtgcagctcgtcgaatccggcggcggcgtggtgcaaccgggacgaagcctgaggctttcttgtgccgcaagcgggtttaccttctccagctacgggatgcactgggtccggcagtctccagggaaggggctggagtgggtcgccgttatctggaatgatggatcctacgtctactacgcagattctgtgaagggacgctttactattagccgcgacatatccaaaaataccctcttcttgcagatgaactcactcagggccgaggataccgccgtttactactgcgcccgggaaggcgagtgggtactgtactattttgactactggggccagggtactcttgtgacggtttctagtagtggggggggtgggagtggcgggggtggtagcggtggcggcggctccgatatccagatgacccagtcgccttcaacactgagcgcttccgtgggagacagagtgactattacatgtagagcgagccagtctattagttcctggctcgcttggtatcaacagaagcccggaaaagctccaaaattattgatatataaggcatcaactctggaaagtggcgtaccctctcgtttctcaggatcaggctccggtaccgagttcaccctgacaatctctagcctgcagcctgacgacttcgctacatattattgccaacagtacaactcgtatagctatacatttggacagggaacgaagctagagatcaaaggcagtaccagcgggtcagggaaacctggcagtggggaaggttccacaaaaggtggttctgaagtgcagttgttagagtcaggaggcgggcttgttcagcctggtggttctctgcggctttcatgcgctgcttccggatttacattctcttcctaccccatgaattgggttcgccaagcccctggaaaaggcttggagtgggtgtcctatatctctcccttcggcggcatgactgattatgctaccagcgtaaagggccggttcactatctctagagacaactcgaagaacacgctctacctacagatgaacagcctgagggccgaagacacggccgtgtattactgtgcgagagatgccatgcgcggggcagaggtggactactggggccagggcaccctggtcacagtatcaagcggcggaggtgggagcggtggggggggtagcgggggaggcggctctgacattcagatgacccagtctccatcctccctgtcagcatctgtgggagacagagtcaccatcacatgccgatccagtcagagcattcccagctatctgaattggtatcagcagaagccagggaaagccccgaagctcctgatttatgctgcatcccgtttacaatccggggtcccatcaaggttcagcggcagtggatcggggacagattttactctcaccataagcagtctgcaacctgaagattttgccacttactactgtcaacagagttacagtacccccctcacttttggcggaggaacaaaggtggagatcaaa

SEQ ID NO: 50 - DNA, 1215 bp, Artificial
  Polynucleotide
atgaagtgggtaacctttatttcccttctttttctctttagctcggcttattccgaagtgcagttgttagagtcaggaggcgggcttgttcagcctggtggttctctgcggctttcatgcgctgcttccggatttacattctcttcctaccccatgaattgggttcgccaagcccctggaaaaggcttggagtgggtgtcctatatctctcccttcggcggcatgactgattatgctaccagcgtaaagggccggttcactatctctagagacaactcgaagaacacgctctacctacagatgaacagcctgagggccgaagacacggccgtgtattactgtgcgagagatgccatgcgcggggcagaggtggactactggggccagggcaccctggtcacagtatcaagcggtggaggtggtagcggtggtggtggtagcgggggaggcggctctgacattcagatgacccagtctccatcctccctgtcagcatctgtgggagacagagtcaccatcacatgccgatccagtcagagcattcccagctatctgaattggtatcagcagaagccagggaaagccccgaagctcctgatttatgctgcatcccgtttacaatccggggtcccatcaaggttcagcggcagtggatcggggacagattttactctcaccataagcagtctgcaacctgaagattttgccacttactactgtcaacagagttacagtacccccctcacttttggcggaggaacaaaggtggagatcaaatctggcggcggcggttctggtggaggaggtagtggggggggaggaagcggaggtggtggctcagggggttctcaggttcagctggtggagtctggcggaggcttggtgcagcccggcgggtcgctgagactctcctgcgctgcctccggattcacctttagtagctactggatgtactgggttaggcaaactccagggaaaggtcttgagtgggtatccactattaatcgggatggtagtgcgacatggtatgcagactcagtgaagggccgattcaccatctcccgcgacaatgccaagaacacaggatatctgcaaatgaacagcttaaaacctgacgacacggctgtgtattactgtgtgagcgatccggataactactctagcgatgaaatggtcccctattggggccaggggacccaggtcacagtctcttca

SEQ ID NO: 51 - DNA, 1602 bp, Artificial
  Polynucleotide
atgaagtgggtaacctttatttcccttctttttctctttagctcggcttattccgaagtgcagttgttagagtcaggaggcgggcttgttcagcctggtggttctctgcggctttcatgcgctgcttccggatttacattctcttcctaccccatgaattgggttcgccaagcccctggaaaaggcttggagtgggtgtcctatatctctcccttcggcggcatgactgattatgctaccagcgtaaagggccggttcactatctctagagacaactcgaagaacacgctctacctacagatgaacagcctgagggccgaagacacggccgtgtattactgtgcgagagatgccatgcgcggggcagaggtggactactggggccagggcaccctggtcacagtatcaagcggtggaggtggtagcggtggtggtggtagcgggggaggcggctctgacattcagatgacccagtctccatcctccctgtcagcatctgtgggagacagagtcaccatcacatgccgatccagtcagagcattcccagctatctgaattggtatcagcagaagccagggaaagccccgaagctcctgatttatgctgcatcccgtttacaatccggggtcccatcaaggttcagcggcagtggatcggggacagattttactctcaccataagcagtctgcaacctgaagattttgccacttactactgtcaacagagttacagtacccccctcacttttggcggaggaacaaaggtggagatcaaatctggcggcggcggttctggtggaggaggtagtggggggggaggaagcggaggtggtggctcagggggttctgatatattactgacccaaactccagcttctttggccgtgtctctggggcagcgcgccaccatctcctgcaaggccagccaatcggttgattatgacggtgacagttacttgaactggtaccaacagattccaggacagccacccaaacttctcatctatgatgcatccaatctggtttcagggataccccccaggtttagtggcagtgggtctgggacagactttacactcaacatccatcccgtggagaaggtggatgctgcgacctatcactgtcagcaaagtactgaagatccgtggacgttcggaggaggcaccaagctggaaatcaaacgaggcggaggtgggagcggtggggggggtagcgggggaggcggctctcaggtgcagctgcagcagtcaggggccgagctggtgcggcctgggagctcagtgaagatttcctgcaaggcttcgggctacgcattcagtagctactggatgaattgggtcaagcagaggcctggacagggccttgagtggattggacagatctggcctggcgacggtgatactaactacaatggaaaatttaagggcaaagccacactgactgcagacgaatcctccagcacagcctacatgcagctcagcagcctagcatccgaggactctgcggtctatttctgtgccagacgggagactacaacggtaggcagatattactatgctatggactactggggtcaaggaacctcagtcacagtatcttca

SEQ ID NO: 52 - DNA, 1602 bp, Artificial; identical to SEQ ID NO: 51
  Polynucleotide

SEQ ID NO: 53 - DNA, 24 bp, Artificial
  Primer
aagagttacactgcctttgccatc

SEQ ID NO: 54 - DNA, 24 bp, Artificial
  Primer
cactgtctgctggtggagttcatc

SEQ ID NO: 55 - DNA, 22 bp, Artificial
  Primer
gggtaccatgaagatctctgca

SEQ ID NO: 56 - DNA, 20 bp, Artificial
  Primer
ttggcggttccttcgagtga

SEQ ID NO: 57 - DNA, 23 bp, Artificial
  Primer
tagtccttcctaccccaatttcc

SEQ ID NO: 58 - DNA, 21 bp, Artificial
  Primer
ttggtccttagccactccttc

SEQ ID NO: 59 - DNA, 21 bp, Artificial
  Primer
aagcctgtagcccacgtcgta

SEQ ID NO: 60 - DNA, 24 bp, Artificial
  Primer
ggcaccactagttggttgtctttg

SEQ ID NO: 61 - DNA, 21 bp, Artificial
  Primer
gctcttactgactggcatgag

SEQ ID NO: 62 - DNA, 20 bp, Artificial
  Primer
cgcagctctaggagcatgtg

SEQ ID NO: 63 - protein, 149 aa, Artificial
  Polypeptide
  misc feature (7)..(7): Ile or Met
  misc feature (8)..(8): Leu or Phe
  CDR1 (44)..(52)
  CDR2 (67)..(83)
  CDR3 (116)..(119)
  misc feature (141)..(141): Ala or Val
MGWSYIXXFLVATDVHSQVQLQQPGAELVKPGASVKLSCKASGYIFTTYWMNWVKQRPGQGLEWIGEISPSDGRINYNEKFKNKATLTVDKSSSTAYMQLSSLTSEDSAVYYCARDSYWGQGTLVTVSAAKTTPPSVYPLXPGSLGRAN

SEQ ID NO: 64 - protein, 9 aa, Artificial
  Polypeptide
YIFTTYWMN

SEQ ID NO: 65 - protein, 17 aa, Artificial
  Polypeptide
EISPSDGRINYNEKFKN

SEQ ID NO: 66 - protein, 4 aa, Artificial
  Polypeptide
DSYW

SEQ ID NO: 67 - protein, 131 aa, Artificial
  Polypeptide
  misc feature (2)..(2): Ile or Thr
  CDR1 (24)..(40)
  CDR2 (56)..(62)
  misc feature (62)..(62): Ser or Pro
  misc feature (71)..(71): Gly or Ser
  misc feature (92)..(92): His or Tyr
  CDR3 (95)..(103)
DXVMSQSPSSLAVSLGEKITMSCKSSQSLLYSSNQKSYLAWYQQKPGQSPELLIYWASTRDXGVPDRFTGXGSGTDFTLTISSVKAEDLAVXFCQQYYTYPYTFGGGTKLEIKRADAAPTVSIFPPSSKLG

SEQ ID NO: 68 - protein, 17 aa, Artificial
  Polypeptide
KSSQSLLYSSNQKSYLA

SEQ ID NO: 69 - protein, 7 aa, Artificial
  Polypeptide
  misc feature (7)..(7): Ser or Pro
WASTRDX

SEQ ID NO: 70 - protein, 9 aa, Artificial
  Polypeptide
QQYYTYPYT